(19) United States
(12) Patent Application Publication
(10) Pub. No.: US 2024/0156825 A1
(43) Pub. Date: May 16, 2024

(54) USE OF SELECTIVE VEGFR2 AND FGFR1 INHIBITORS

(71) Applicant: Fountain Therapeutics Inc., South San Francisco, CA (US)

(72) Inventors: Joseph T. Rodgers, Redwood City, CA (US); David H. Le, Daly City, CA (US); Rajat Bhatnagar, South San Francisco, CA (US); Jesse A. Rosen, Jersey City, NJ (US); Fernando J. Martinez, Burlingame, CA (US)

(21) Appl. No.: 18/332,357

(22) Filed: Jun. 9, 2023

Related U.S. Application Data
(60) Provisional application No. 63/351,254, filed on Jun. 10, 2022.

(51) Int. Cl. A61K 31/53; A61K 31/335; A61K 31/352; A61K 31/404; A61K 31/405; A61K 31/4155; A61K 31/427; A61K 31/4375; A61K 31/44; A61K 31/454; A61K 31/496; A61K 31/517; A61K 31/519; A61K 31/5377; A61K 31/55; A61K 31/5517; A61K 45/06; A61P 1/16; A61P 19/00

(52) U.S. Cl. CPC A61K 31/53; A61K 31/335; A61K 31/352; A61K 31/404; A61K 31/405; A61K 31/4155; A61K 31/427; A61K 31/4375; A61K 31/44; A61K 31/454; A61K 31/496; A61K 31/517; A61K 31/519; A61K 31/5377; A61K 31/55; A61K 31/5517; A61K 45/06; A61P 1/16; A61P 19/00

(57) ABSTRACT

The disclosure relates to methods of using selective inhibitors of VEGFR2 and FGFR1 to promote lipid storage and fat increase, and for treatment of a wasting syndrome.

### CROSS-REFERENCE TO RELATED APPLICATIONS

This application claims the benefit of and priority to U.S. Provisional Patent Application No. 63/351,254, filed Jun. 10, 2022, the entire contents of each of which are incorporated by reference herein for all purposes.

### FIELD OF THE EMBODIMENTS

The present disclosure relates to the use of selective VEGFR2 and FGFR1 inhibitors for the promotion of lipid storage or fat mass increase in a subject, and the treatment of wasting syndrome in a subject.

### BACKGROUND OF THE EMBODIMENTS

Cachexia, or wasting syndrome, is a condition that causes extreme weight loss and muscle wasting in several associated disorders and diseases. Cachexia affects thousands of people every year. There is a lack of existing, successful treatments that can be prescribed for treating cachexia, hence the need for the development of therapies that can aid subjects exhibiting a wasting syndrome.

Herein, we described methods to promote lipid storage or increase of fat mass in a subject, and the treatment of cachexia and its other sequelae in a subject using selective inhibitors of VEGFR2 and FGFR1.

### SUMMARY

In the present disclosure, we describe methods directed to the use of compounds that selectively inhibit both Vascular endothelial growth factor receptor 2 (VEGFR2) and fibroblast growth factor receptor 1 (FGFR1) (i.e., a selective inhibitor of VEGFR2 and FGFR1) to promote lipid storage or fat mass increase in a subject, and for the treatment of a wasting syndrome in a subject.

In some embodiments, there is a method of preserving or promoting lipid storage in subcutaneous adipose tissue, fat mass increase, brown fat mass increase, bone density, lean muscle mass increase, or the thickening of abdominal subcutaneous adipose tissue, or decreasing liver lipid content in a subject comprising administering to the subject a selective inhibitor of VEGFR2 and FGFR1, wherein the administration promotes the lipid storage or fat mass increase in the subject as compared to administration of a control agent or as compared to historical data obtained from the subject prior to the administration of the selective inhibitor of VEGFR2 and FGFR1.

In some embodiments, provided is a method of preserving or promoting lipid storage in subcutaneous adipose tissue, fat mass increase, brown fat mass increase, bone density, lean muscle mass increase, or the thickening of abdominal subcutaneous adipose tissue, or decreasing liver lipid content in a subject comprising administering to the subject a selective inhibitor of VEGFR2 and a selective inhibitor of FGFR1, wherein the administration promotes the lipid storage or fat mass increase in the subject as compared to administration of a control agent or as compared to historical data obtained from the subject prior to the administration of the selective inhibitor of VEGFR2 and a selective inhibitor FGFR1.

In some embodiments, the subject does not exhibit a cancer-associated cachexia.

In some embodiments, the subject exhibits a wasting syndrome.

In some embodiments, the wasting syndrome is selected from the group consisting of lipoatrophy, cachexia, liver disease, kidney disease, fatty liver disease, Non-alcoholic Steato Hepatitis (NASH), diabetes, osteoporosis, Paget's disease of the bone, osteoarthritis, rheumatoid arthritis, sarcopenia, and frailty.

In some embodiments, provided is a method of treating, preventing, reversing, or delaying progression of a wasting syndrome in a subject comprising administering to the subject a selective inhibitor of VEGFR2 and FGFR1.

In some embodiments, provided is a method of treating, preventing, reversing, or delaying progression of a wasting syndrome in a subject comprising administering to the subject a selective inhibitor of VEGFR2 and FGFR1.

In some embodiments, the method comprises treating, reversing, or delaying progression of the wasting syndrome in the subject.

In some embodiments, the wasting syndrome is selected from the group consisting of lipoatrophy, cachexia, liver disease, kidney disease, fatty liver disease, Non-alcoholic Steato Hepatitis (NASH), diabetes, osteoporosis, Paget's disease of the bone, osteoarthritis, rheumatoid arthritis, sarcopenia, and frailty.

In some embodiments, the selective inhibitor of inhibitor of VEGFR2 and FGFR1 is administered as a mono-drug therapy.

In some embodiments, the selective VEGFR2 and FGFR1 inhibitor is administered 6 times/day, 4 times/day, 3 times/day, 2 times/day, once per day, once every 2 days, once every 3 days, once every 4 days, once every 5 days, once every 6 days, or once per week.

In some embodiments, the selective VEGFR2 and FGFR1 inhibitor is selected from the group consisting of [4-(2,4-Difluoro-5-methoxycarbamoyl-phenylamino)-5-isopropyl-pyrrolo[2,1-f][1,2,4]triazin-6-yl]-carbamic acid tetrahydro-furan-2-ylmethyl ester, Brivanib Alaninate (Brivanib), 2,4-Difluoro-5-[5-isopropyl-6-(5-trifluoromethyl-[1,3,4]oxadiazol-2-yl)-pyrrolo[2,1-f][1,2,4]triazin-4-ylamino]-N-methoxy- benzamide, 5-[(1-Ethylpiperidin-4-yl)amino]-3-[1H-imidazol-2-yl(phenyl)methylidene]-1H-indol-2-one, 2,4-Difluoro-5-[5-isopropyl-6-(5-methyl-4H-[1,2,4]triazol-3-yl)-pyrrolo[2,1-f][1,2,4]triazin-4-ylamino]-N-methoxy-benzamide, 5-[6-(5-Dimethylamino-[1,3,4]oxadiazol-2-yl)-5-isopropyl-pyrrolo[2,1-f][1,2,4]triazin-4-ylamino]-2,4-difluoro-N-methoxy-benzamide, 2,4-Difluoro-5-[5-isopropyl-6-(3-methyl-[1,2,4]oxadiazol-5-yl)-pyrrolo[2,1-f][1,2,4]triazin-4-ylamino]-N-methoxy-benzamide, 3-[(3-(2- Carboxyethyl)-4-methylpyrrol-2-YL)methylene]-2-indolinone (SU5402), 2,4-Difluoro-5-[5-isopropyl-6-(5-methyl-[1,3,4]oxadiazol-2-yl)-pyrrolo[2,1-f][1,2,4]triazin-4-ylamino]-N-methoxy-benzamide, 5-[6-(5-Difluoromethyl-[1,3,4]oxadiazol-2-yl)-5-isopropyl-pyrrolo[2,1-f][1,2,4]triazin-4-ylamino]-2,4-difluoro-N-methoxy-benzamidem, 3-[Benzimidazol-2-ylidene-(3-fluorophenyl)methyl]-5-[(1-ethylpiperidin-4-yl)amino]-1H-indol-2-ol, N-(Cyclopropylmethyl)-3-(1-methylpyrazol-4-yl)-N-phenylquinoxalin-6-amine, 2,4-Difluoro-5-(5-isopropyl-6-[1,3,4]oxadiazol-2-yl-pyrrolo[2,1-f][1,2,4]triazin-4-ylamino)-N-methoxy-benzamide, 7-(4-Fluoroanilino)-1-[(1R,3R)-3-hydroxycyclopentyl]-3-(4-methoxyphenyl)-4H-pyrimido[4,5-d]pyrimidin-2-one, 5-[6-(5-Cyclopropyl-[1,3,4]oxadiazol-2-yl)-5-isopropyl-pyrrolo[2,1-f][1,2,4]triazin-4-ylamino]-2,4-difluoro-N-methoxy-benzamide, 6-(5-Methyl-1,3,4-oxadiazol-2-yl)-5-propan-2-yl-N-(1H-pyrrolo[2,3-b]pyridin-5-yl)pyrrolo[2,1-f][1,2,4]triazin-4-amine, N-(Cyclopropylmethyl)-N-[3-methoxy-5-(trifluoromethyl)phenyl]-3-(1-methylpyrazol-4-yl)quinoxalin-6-amine, 3-[Benzimidazol-2-ylidene-(3,5-difluorophenyl)methyl]-5-[(1-ethylpiperidin-4-yl)amino]-1H-indol-2-ol, 1,6-Naphthyridine 76, 2,4-Difluoro-5-{5-isopropyl-6-[5-(2,2,2-trifluoro-ethyl)-[1,3,4]oxadiazol-2-yl]-pyrrolo[2,1-f][1,2,4]triazin-4- ylamino}-N-methoxy-benzamide, (+)-(1R,3R)-1-(3-Hydroxy-cyclopentyl)-3-(4-methoxy-phenyl)-7-phenylamino-3,4-dihydro-1H-pyrimido[4,5-d]pyrimidin-2-one, 1,6-Naphthyridine 75, (−)-(1 S,3 S)-1-(3-Hydroxy-cyclopentyl)-3-(4-methoxy-phenyl)-7-phenylamino-3,4-dihydro-1H-pyrimido[4,5-d]pyrimidin-2-one, N-(Cyclopropylmethyl)-N-[3-[(dimethylamino)methyl]-5-methoxyphenyl]-3-(1-methylpyrazol-4-yl)quinoxalin-6-amine, N-[3-(Aminomethyl)-5-methoxyphenyl]-N-(cyclopropylmethyl)-3-(1-methylpyrazol-4-yl)quinoxalin-6-amine;hydrochloride, Chemb14573547, 1,6-Naphthyridine 77, 1,6-Naphthyridine 19, 1,6-Naphthyridine 26, N-(Cyclopropylmethyl)-N-[3-(difluoromethoxy)phenyl]-3-(1-methylpyrazol-4-yl)quinoxalin-6-amine, N′-(3,4-Difluoro-5-methoxyphenyl)-N′-[3-(1-methylpyrazol-4-yl)quinoxalin-6-yl]-N-(2,2,2-trifluoroethyl)propane-1,3-diamine;hydrochloride, 2,4-Difluoro-5-[5-isopropyl-6-(5-methanesulfonylmethyl-[1,3,4]oxadiazol-2-yl)-pyrrolo[2,1-f][1,2,4]triazin-4-ylamino]-N-methoxy-benzamide, 5-(2-Chlorophenyl)-7-fluoro-1,2-dihydro-8-methoxy-3-methylpyrazolo(3,4-b)(1,4)benzodiazepine, [3-[Cyclopropylmethyl-[3-(1-methylpyrazol-4-yl)quinoxalin-6-yl]amino]-5-methoxyphenyl]-pyrrolidin-1-ylmethanone, 5-[(1-Ethylpiperidin-4-yl)amino]-3-[(2-fluorophenyl)-(5-methyl-1H-imidazol-2-yl)methylidene]-1H-indol-2-one, 5-[6-(5-Cyclopropylmethyl-[1,3,4]oxadiazol-2-yl)-5-isopropyl-pyrrolo[2, 1-f][1,2,4]triazin-4-ylamino]-2,4-difluoro-N-methoxy-benzamide, 3-[(3,5-Difluorophenyl)-(5-methyl-1H-imidazol-2-yl)methylidene]-5-[(1-ethylpiperidin-4-yl)amino]-1H-indol-2-one, 5-[(1-Ethylpiperidin-4-yl)amino]-3-[(3-fluorophenyl)-(1H-imidazol-2-yl)methylidene]-1H-indo1-2-one, Indolin-2-one deriv. 9c, Methyl (3Z)-2-oxo-3-[phenyl-[4-(piperidin-1-ylmethyl)anilino]methylidene]-1H-indole-6-carboxylate, N-(3,5-Dimethoxyphenyl)-N-[3-(3-methylimidazol-4-yl)prop-2-ynyl]-3-(1-methylpyrazol-4-yl)quinoxalin-6-amine, (3z)-5-[(1-Ethylpiperidin-4-Yl)amino]-3-[(5-Methoxy-1h-Benzimidazol-2-Yl)(Phenyl)methylidene]-1,3-Dihydro-2h-Indol-2-One, Chemb14449189, [5-Amino-1-(1H-indo1-5-yl)pyrazol-4-yl]-(1H-indol-2-yl)methanone, 5-Isopropyl-6-(5-methyl-1,3,4-oxadiazol-2-yl)-N-(2-methyl-1H-pyrrolo[2,3-b]pyridin-5-yl)pyrrolo[2,1-f][1,2,4]triazin-4-amine, 3-[Benzimidazol-2-ylidene-(4-methylphenyl)methyl]-5-[(1-ethylpiperidin-4-yl)amino]-1H-indo1-2-ol, 5-[6-(5-Ethyl-[1,3,4]oxadiazol-2-yl)-5-isopropyl-pyrrolo[2,1-f][1,2,4]triazin-4-ylamino]-2,4-difluoro-N-methoxy-benzamide, 3-[Benzimidazol-2-ylidene(phenyl)methyl]-5-[(1-ethylpiperidin-4-yl)amino]-1H-indol-2-ol, 1,6-Naphthyridine 25, 2-(6-Fluoro-1H-indazol-3-yl)-5-(4-(piperidin-1-yl)piperidin-1-yl)-1H-benzo[d]imidazole, 3-[Benzimidazol-2-ylidene-(4-methoxyphenyl)methyl]-5-[(1-ethylpiperidin-4-yl)amino]-1H-indol-2-ol, N-(Cyclopropylmethyl)-N-[3-methoxy-5-[2-(methylamino)ethoxy]phenyl]-3-(1-methylpyrazol-4-yl)quinoxalin-6-amine, 3-Methoxy-N-methyl-5-[[3-(1-methylpyrazol-4-yl)quinoxalin-6-yl]-[2-(propan-2-ylamino)ethyl]amino]benzamide, XL999;XL-999; XL 999, 3-N-(Cyclopropylmethyl)-1-N-methyl-3-N-[3-(1-methylpyrazol-4-yl)quinoxalin-6-yl]benzene-1,3-diamine, 2,4-Difluoro-5-[6-(5-isobutyl-[1,3,4]oxadiazol-2-yl)-5-isopropyl-pyrrolo[2,1-f][1,2,4]triazin-4-ylamino]-N-methoxy-benzamide, Methyl 2-hydroxy-3-[N-[4-[2-methyl-5-[(4-methylpiperazin-1-yl)methyl]pyrrol-1-yl]phenyl]-C-phenylcarbonimidoyl]-1H-indole-6-carboxylate, N-(3,5-Dimethoxyphenyl)-3-(1-methylpyrazol-4-yl)-N-[(2-pyridin-4-ylcyclopropyl)methyl]quinoxalin-6-amine, 3-(3,5-Dimethoxyphenyl)-7-N-[3-(4-methylpiperazin-1-yl)propyl]-1,6-naphthyridine-2,7-diamine, 9-Chloro-2-[3-[3-(dimethylamino)propyl]anilino]-5,7-dihydropyrimido[5,4-d][1]benzazepine-6-thione, Pyrido[2,3-d]pyrimidine 103, Pyrido[2,3-d]pyrimidine 105, 2,4-Difluoro-5-[5-isopropyl-6-(5-methyl-oxazol-2-yl)-pyrrolo[2,1-f][1,2,4]triazin-4-ylamino]-N-methoxy-benzamide, N-(3,5-Dimethoxyphenyl)-N-[3-(2,5-dimethylimidazol-1-yl)propyl]-3-(1-methylpyrazol-4-yl)quinoxalin-6-amine;hydrochloride, N-[3-[2-[6-(2-Chlorophenyl)-2-[4-(diethylamino)butylamino]-7-oxopyrido[2,3-d]pyrimidin-8-yl]ethyl]phenyl]prop-2-enamide, N-(3,5-Dimethoxyphenyl)-3-(1-methylpyrazol-4-yl)-N-(2-piperazin-1-ylethyl)quinoxalin-6-amine;hydrochloride, Methyl (3Z)-3-[[4-[2-(dimethylamino)ethyl-methylsulfonylamino]anilino]-phenylmethylidene]-2-oxo-1H-indole-6-carboxylate, 4-[3-[3,5-Dimethoxy-N-[3-(1-methylpyrazol-4-yl)quinoxalin-6-yl]anilino)prop-1-ynyl]pyridine-3-carbonitrile, N-[(5-Chlorothiophen-2-yl)methyl]-N-(3,5-dimethoxyphenyl)-3-[1-(2-piperazin-1-ylethyl)pyrazol-4-yl]quinoxalin-6-amine;hydrochloride, N′-(3,5-Dichlorophenyl)-N′-[3-(1-methylpyrazol-4-yl)quinoxalin-6-yl]-N-(2,2,2-trifluoroethyl)propane-1,3-diamine, 2-[4-[7-[N-(Cyclopropylmethyl)anilino]quinoxalin-2-yl]pyrazol-1-yl]ethanol, Pyrido[2,3-d]pyrimidine 102, 7-N-[4-(Diethylamino)butyl]-3-(3,5-dimethoxyphenyl)-1,6-naphthyridine-2,7-diamine, N-(3,5-Dimethoxyphenyl)-N-[3-(4-methoxypyrimidin-2-yl)propyl]-3-(1-methylpyrazol-4-yl)quinoxalin-6-amine, Methyl 3-[[4-[(dimethylamino)methyl]anilino]-phenylmethylidene]-2-oxo-1H-indole-6-carboxylate, 3-Fluoro-N-methyl-5-[[3-(1-methylpyrazol-4-yl)quinoxalin-6-yl]-[2-(propan-2-ylamino)ethyl]amino]benzamide, N-(Cyclopropylmethyl)-N-[3-(1,3-dioxolan-2-yl)-5-methoxyphenyl]-3-(1-methylpyrazol-4-yl)quinoxalin-6-amine, N-[[(2S)-1-[3-(3,5-Dimethoxy-N-[3-(1-methylpyrazol-4-yl)quinoxalin-6-yl]anilino)propyl]pyrrolidin-2-yl]methyl]-1,1,1-trifluoromethanesulfonamide, 3-Methoxy-N-methyl-5-[2-(propan-2-ylamino)ethyl-[3-(1-propan-2-ylpyrazol-4-yl)quinoxalin-6-yl]amino]benzamide, 5-{6-[5-(Difluoro-methanesulfonyl-methyl)-[1,3,4]oxadiazol-2-yl]-5-isopropyl-pyrrolo[2,1-f][1,2,4]triazin-4-ylamino}-2,4-difluoro-N-methoxy- benzamide, N-[2-(3-Aminopiperidin-1-yl)ethyl]-N-(3,5-dimethoxyphenyl)-3-(1-methylpyrazol-4-yl)quinoxalin-6-amine, N-(3,5-Dimethoxyphenyl)-N-[(E)-3-(4-methoxypyrimidin-2-yl)prop-2-enyl]-3-(1-methylpyrazol-4-yl)quinoxalin-6-amine, Methyl (3Z)-3-[[4-[acetyl-[2-(dimethylamino)ethyl]amino]anilino]-phenylmethylidene]-2-oxo-1H-indole-6-carboxylate, Methyl N-[4-[7-amino-3-(3,4-dimethoxyphenyl)-5-(1-methylpiperidin-4-yl)oxypyrazolo[1,5-a]pyrimidin-6-yl]phenyl]carbamate, Anilinoquinazoline deriv. 35, 1,6-Naphthyridine 17, Tert-butyl (1S,4S)-5-[2-(3,5-dimethoxy-N-[3-(1-methylpyrazol-4-yl)quinoxalin-6-yl]anilino)ethyl]-2,5-diazabicyclo[2.2.1]heptane-2- carboxylate, Lucitanib, 6-((7-((1-Aminocyclopropyl)methoxy)-6-methoxyquinolin-4-yl)oxy)-N-methyl-1-naphthamide hydrochloride, Pyrido[2,3-d]pyrimidine 104, Nintedanib esylate, [(2S)-1-(3,5-Dimethoxy-N-[3-(1-methylpyrazol-4-yl)quinoxalin-6-yl]anilino)-3-methoxypropan-2-yl] acetate, 3-[2-Aminoethyl-[3-(1-methylpyrazol-4-yl)quinoxalin-6-yl]amino]-5-methoxy-N-methylbenzamide, N-(3,5-Dimethoxyphenyl)-3-(1-methylpyrazol-4-yl)-N-[3-[4-(trifluoromethyl)piperidin-1-yl]propyl]quinoxalin-6-amine, 3-((4-Bromo-2,6-difluorobenzyl)oxy)-5-(3-(4-(pyrrolidin-1-yl)butyl)ureido)isothiazole-4-carboxamide, N-(3,5-Dimethoxyphenyl)-N-[3-(2-methylimidazol-1-yl)propyl]-3-(1-methylpyrazol-4-yl)quinoxalin-6-amine, 3-[3-(Morpholinomethyl)-4,5,6,7-tetrahydro-1H-indole-2-ylmethylene]-5-(ethylsulfonyl)-2,3-dihydro-1H-indole-2-one, 2-(3-Methoxy-N-[3-(1-methylpyrazol-4-yl)quinoxalin-6-yl]anilino)ethanol, Epihematoxylol, N-[3-(5-Aminopyrazin-2-yl)prop-2-ynyl]-N-(3,5-dimethoxyphenyl)-3-(1-methylpyrazol-4-yl)quinoxalin-6-amine, 2-[3-[2-Aminoethyl-[7-(1-methylpyrazol-4-yl)quinoxalin-2-yl]amino]-5-fluorophenoxy]ethanol, N-[3-(3-Aminopyridin-2-yl)prop-2-ynyl]-N-(3,5-dimethoxyphenyl)-3-(1-methylpyrazol-4-yl)quinoxalin-6-amine, N-(3,5-Dimethoxyphenyl)-N-[3-(3-methoxypyridin-2-yl)propyl]-3-(1-methylpyrazol-4-yl)quinoxalin-6-amine, N-(3,5-Dimethoxyphenyl)-3-(1-methylpyrazol-4-yl)-N-[(Z)-3-pyrimidin-2-ylprop-2-enyl]quinoxalin-6-amine, N′-(3,5-Dimethoxyphenyl)-N-methyl-N′-[3-[1-(oxan-4-ylmethyl)pyrazol-4-yl]quinoxalin-6-yl]ethane-1,2-diamine;hydrochloride, [5-Amino-1-(2-chloro-5-hydroxyphenyl)pyrazol-4-yl]-[5-(morpholin-4-ylmethyl)-1H-indol-2-yl]methanone, 1,6-Naphthyridine 18, 1-Tert-butyl-3-[3-(3,5-dimethoxyphenyl)-7-[4-(4-methylpiperazin-1-yl)butylamino]-1,6-naphthyridin-2-yl]urea, N′-(2-Chloro-3,5-dimethoxyphenyl)-N′-[3-(1-methylpyrazol-4-yl)quinoxalin-6-yl]-N-propan-2-ylethane-1,2-diamine;hydrochloride, N-(3,5-Dimethoxyphenyl)-3-(1-methylpyrazol-4-yl)-N-(3-piperazin-1-ylpropyl)quinoxalin-6-amine, Methyl (3Z)-3-[[4-[acetyl-[3-(dimethylamino)propyl]amino]anilino]-phenylmethylidene]-2-oxo-1H-indole-6-carboxylate, 3-[4-[3-[4-[7-[N-(Cyclopropylmethyl)-3,5-dimethoxyanilino]quinoxalin-2-yl]pyrazol-1-yl]propyl]piperazin-1-yl]propan-1-ol;hydrochloride, 5-[(R)-1-(3,5-Dichloropyridine-4-yl)ethoxy]-3-[5-(4-methylpiperazine-1-yl)-1H-benzoimidazole-2-yl]-1H-indazole, 4-Amino-3-(1H-benzo[d]imidazol-2-yl)quinolin-2(1H)-one, 3-[4-[3-[4-[7-[3,5-Dimethoxy-N-[2-(propan-2-ylamino)ethyl]anilino]quinoxalin-2-yl]pyrazol-1-yl]propyl]piperazin-1-yl]propan-1- ol;hydrochloride, 2-(2,6-Difluoro-3,5-dimethoxy-N-[3-(1-methylpyrazol-4-yl)quinoxalin-6-yl]anilino)ethanol, [4-[7-[3,5-Dimethoxy-N-[2-(propan-2-ylamino)ethyl]anilino]quinoxalin-2-yl]-2-methylpyrazol-3-yl]methanol, 1,6-Naphthyridine deriv. 20, N-(2-Chloro-3,5-dimethoxyphenyl)-3-(1-methylpyrazol-4-yl)-N-(3-pyrrolidin-1-ylpropyl)quinoxalin-6-amine;hydrochloride, Methyl 3-[N-[4-[2-(dimethylamino)ethyl-methylcarbamoyl]phenyl]-C-phenylcarbonimidoyl]-2-hydroxy-1H-indole-6-carboxylate, N-[2-(1,4-Diazepan-1-yl)ethyl]-N-(3,5-dimethoxyphenyl)-3-(1-methylpyrazol-4-yl)quinoxalin-6-amine; oxalic acid, N′-(2,6-Difluoro-3,5-dimethoxyphenyl)-N′-[3-(1-methylpyrazol-4-yl)quinoxalin-6-yl]-N-propan-2-ylethane-1,2-diamine, N-(3,5-Dimethoxyphenyl)-N-[(3-methyloxetan-3-yl)methyl]-3-(1-methylpyrazol-4-yl)quinoxalin-6-amine, Methyl (3Z)-3-[[4acetyl-[2-(dimethylamino)-2-oxoethyl]amino]anilino]-phenylmethylidene]-2-oxo-1H-indole-6-carboxylate, Chemb14213341, Anilinoquinazoline deriv. 36, [5-Amino-1-(1H-benzimidazol-6-yl)-1H-pyrazol-4-yl]-1H-indo1-2-ylMethanone, Hematoxyl one, N-Cyclopropyl-N′-(3,5-dimethoxyphenyl)-N′-[3-(1-methylpyrazol-4-yl)quinoxalin-6-yl]ethane-1,2-diamine, N-(3,5-Dimethoxyphenyl)-3-(1-methylpyrazol-4-yl)-N-(3-pyrimidin-2-ylpropyl)quinoxalin-6-amine, N′-(3,5-Dimethoxyphenyl)-N′-[3-[5-[(dimethylamino)methyl]-1-methylpyrazol-4-yl]quinoxalin-6-yl]-N-propan-2-ylethane-1,2-diamine, N′-(2,6-Difluoro-3,5-dimethoxyphenyl)-N′-[3-(1-methylpyrazol-4-yl)quinoxalin-6-yl]-N-(2,2,2-trifluoroethyl)propane-1,3-diamine, [5-Amino-1-(2-methyl-1H-indol-5-yl)pyrazol-4-yl]-(1H-indol-2-yl)methanone, Haematoxylin, N′-(3,5-Dimethoxyphenyl)-N′-[3-(5-ethyl-1-methylpyrazol-4-yl)quinoxalin-6-yl]-N-(2,2,2-trifluoroethyl)ethane-1,2-diamine, Methyl 3-[N-[4-[[dimethylcarbamoyl(methyl)amino]methyl]phenyl]-C-phenylcarbonimidoyl]-2-hydroxy-1H-indole-6-carboxylate, 3-Methoxy-N-methyl-5-[[3-(1-methylpyrazol-4-yl)quinoxalin-6-yl]-(3-pyrrolidin-1-ylpropyl)amino]benzamide, N-(3,5-Dimethoxyphenyl)-3-(1-methylpyrazol-4-yl)-N-(3-pyridin-4-ylpropyl)quinoxalin-6-amine, 1-[3-(3,5-Dimethoxy-N-[3-(1-propan-2-ylpyrazol-4-yl)quinoxalin-6-yl]anilino)propyl]pyrrolidin-2-one, and Regorafenib.

In some embodiments, the selective VEGFR2 and FGFR1 inhibitor is Brivanib Alaninate (Brivanib).

In some embodiments, Brivanib Alaninate is administered to the subject at a daily dose of 10 mg/kg-200 mg/kg.

In some embodiments, Brivanib Alaninate is administered to the subject at a daily dose of 25 mg/kg.

In some embodiments, Brivanib Alaninate is administered to the subject at a daily dose of 50 mg/kg.

In some embodiments, the selective VEGFR2 and FGFR1 inhibitor is SU5402.

In some embodiments, SU5402 is administered to the subject at a daily dose of 5 mg/kg-100 mg/kg.

In some embodiments, SU5402 is administered to the subject at a daily dose of 25 mg/kg.

In some embodiments, the selective VEGFR2 or FGFR1 inhibitor or the selective inhibitor of VEGFR2 and FGFR1 is administered for up to 3 months.

In some embodiments, the selective VEGFR2 or FGFR1 inhibitor or the selective inhibitor of VEGFR2 and FGFR1 is administered for up to 2 months.

In some embodiments, the selective VEGFR2 or FGFR1 inhibitor or the selective inhibitor of VEGFR2 and FGFR1 is administered for up to 1 month.

In some embodiments, the selective VEGFR2 or FGFR1 inhibitor or the selective inhibitor of VEGFR2 and FGFR1 is administered by a route selected from the group consisting of topically, intrathecally, intrathalamically, intracisternally, parenterally, orally, rectally, buccally, sublingually, pulmonarily, intratracheally, intranasally, transdermally, and intraduodenally.

### BRIEF DESCRIPTION OF THE DRAWINGS

FIGURE (FIG. 1A. Body fat of Zmpste24 mice treated with Brivanib Alaninate or vehicle.

FIG.1B. H&E stained intrascapular brown adipose tissue from 15 week old male Zmpste24−/− mice treated with Brivanib Alaninate or vehicle.

FIG. 1C. Intrascapular brown fat mass measured from 15 week old male Zmpste24−/− mice treated with Brivanib Alaninate or vehicle.

FIG. 2A. Bone loss of vertebrae from H&E stained sections of Zmpste24−/− mice treated with Brivanib Alaninate or vehicle.

FIG. 2B. Bone loss of femur scored from H&E stained sections of Zmpste24−/− mice treated with Brivanib Alaninate or vehicle.

FIG. 3A. Images of H&E stained liver samples from mice with diet induced fatty liver disease, treated with Brivanib Alaninate or vehicle.

FIG. 3B. Images of H&E stained adipocytes in the epididymal fat pad samples from mice with diet induced fatty liver disease, treated with Brivanib Alaninate or vehicle.

FIG. 3C. Images of H&E stained abdominal subcutaneous adipose tissue samples from mice with diet induced fatty liver disease, treated with Brivanib Alaninate or vehicle.

FIG. 3D-H. Body weight, liver mass, serum cholesterol, serum triglycerides, and blood urea nitrogen measured from 26 week old mice fed with a standard diet or with diet induced fatty liver disease and treated with Brivanib Alaninate or vehicle. Color key for bar graphs is in FIG. 3H.

FIG. 4A-C. Body fat, lean muscle mass, and body weight of Zmpste24^{-/-} mice treated with SU5402 or vehicle.

FIG. 4D. Intrascapular brown fat mass measured from 15 week old male Zmpste24−/− mice treated with SU5402 or vehicle.

FIG. 5A-C. Body fat, muscle mass, and body weight of Zmpste24^{-/-} mice (g) treated with 5 mg/kg Cediranib Maleate or vehicle over time of treatment.

FIG. 5D. Intrascapular brown fat mass measured from 11 week old female Zmpste24−/− mice treated with Cediranib Maleate or vehicle.

FIG. 6A-C. Body fat, muscle mass, and body weight of Zmpste24^{-/-} mice treated with 10 mg/kg Regorafenib or vehicle.

FIG. 6D. Intrascapular brown fat mass measured from 11 week old female Zmpste24−/− mice treated with Regorafenib or vehicle.

FIG. 7A-C. Body fat, muscle mass, and body weight of Zmpste24 mice treated with 10 mg/kg Infigratinib or vehicle over time of treatment.

FIG. 7D. Intrascapular brown fat mass measured from 9 week old male Zmpste24−/− mice treated with Infigratinib or vehicle.

### DETAILED DESCRIPTION OF THE EMBODIMENTS

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

As used herein, the terms “treat” or “treatment” are used interchangeably and are meant to indicate administering one or more compounds in accordance with the methods of the embodiments to assist in treatment of a wasting syndrome or promote lipid storage to obtain a desired therapeutic objective. The term “treatment” as used herein covers any treatment of a disease in a mammal, particularly a human, and includes: (a) inhibiting the disease, i.e. arresting its development; or (b) relieving the disease, i.e. causing regression of the disease and/or its symptoms or conditions. The treatment may be therapeutic in terms of a partial or complete cure of a disease, condition, symptom or adverse effect attributed to the disease.

The term “preventing” is meant to indicate a prophylactic effect in terms of preventing or partially preventing a disease, symptom or condition thereof. The term “preventing” includes preventing the disease occurring in a subject which may be predisposed to the disease nut has not yet been diagnosed.

The term “effective amount,” including related terms “prophylaxis-effective amount,” “treatment effective amount,” and “therapeutically effective amount,” refers to a concentration of a compound that is effective in promoting lipid storage or fat mass increase in a subject or prevent or treat (respectively, as will be clear from context in this specification) of a wasting syndrome in a subject.

The term “prevention” as used herein, including the related terms such as “prevent” or “preventing,” is meant to refer to provide a subject not yet affected by the condition with a benefit that serves to avoid, delay, forestall, minimize, or reduce the recurrence/onset of the condition to be prevented and/or its attendant symptoms. Such preventive benefits include, for example, delaying development and/or recurrence of the condition, or reducing the duration, severity, or intensity of one or more unwanted features associated with the condition if it eventually develops.

As used herein, the term “small molecule” is meant to indicate a chemical compound having a molecular weight of less than about 500 Daltons. Small molecules do not include biologic polymer such as polypeptides and polynucleotides.

The term “pharmaceutically acceptable” or “pharmacologically acceptable” is meant a material which is not biologically or otherwise undesirable, i.e. the material may be administered to an individual without causing any undesirable biological effects or interacting in a deleterious manner with any of the components of the composition in which it is contained.

The term “physiological pH” or a “pH in the physiological range” is meant a pH in the range of approximately 7.2 to 8.0 inclusive, more typically in the range of approximately 7.2 to 7.6 inclusive.

As used herein, the term “subject” encompasses mammals. Examples of mammals include, but are not limited to, any member of the Mammalia class: humans, non-human primates, such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

The term “about” is used when determinants or values do not need to be identical, i.e. 100% the same. Accordingly, “about” means, that a determinant or values may diverge by 0.1% to 20%, preferably by 0.1% to 10%; in particular, by 0.5%, 1%, 2%, 3%, 4%, 5%, 6%, 7%, 8%, 9%, 10%. The skilled person will know that certain parameters or determinants may slightly vary based on the method how the parameter was determined.

The terms “selective inhibitor of VEGFR2 and FGFR1” and “selective VEGFR2 and FGFR1 inhibitors” are used herein to mean a small molecule that targets VEGFR2 and FGFR1 with a reported IC₅₀ for each of the targets of less than or equal to 10 μM. These compounds are also referred to as compounds that selectively inhibit both VEGFR2 and FGFR1.

The term “selective inhibitor of VEGFR2” as used herein means a small molecule that targets VEGFR2, and has a relative IC₅₀ value below 1.

The term “selective inhibitor of FGFR1” as used herein means a small molecule that targets FGFR1 and has a relative IC₅₀ value below 1.

The “relative IC₅₀” is the calculation of the ratio of the reported IC₅₀ towards either VEGFR2 or FGFR1 to the next lower reported IC₅₀ to any gene target.

Non-limiting examples of liver diseases include fatty liver disease, Non-alcoholic Steatohepatitis (Nash), liver cancers. Acute hepatic porphyria, Alagille syndrome, Alcohol-related liver disease, Alpha-1 Antitrypsin Deficiency, Autoimmune Hepatitis, Bilde Duct Cancer, Biliary Atresia, Budd-Chiari Syndrome, Crigler-Najjar Syndrome, Galactosemia, Gilbert Syndrome, Glycogen Storage Disease Type 1, Hemochromatosis, Hepatitis A, Hepatitis B, Hepatitis C, Hepatorenal Syndrome, Intrahepatic Cholestasis of Pregnancy (ICP), Lysosomal Acid Lipase Deficiency (LAL-D), Non-Alcoholic Fatty Liver Disease, Primary Biliary Cholangitis (PBC), Primary Sclerosing Cholangitis, Progressive Familial Intrahepatic Cholestasis (PFIC), Reye Syndrome, and Wilson Disease.

Non-limiting examples of kidney diseases include diabetes, kidney cancer, chronic kidney disease, polycystic kidney disease, Fabry disease, Cystinosis, Glomerulonephritis, IgA nephropathy, Lupus Nephritis, and atypical hemolytic uremic syndrome (aHUS).

### Medical Significance

Cachexia, or wasting syndrome, is a condition that causes extreme weight loss and muscle wasting. It is a symptom of many chronic conditions such as cancer, chronic renal failure, HIV, multiple sclerosis, heart disease, liver diseases, kidney diseases, osteoporosis, arthritis, sarcopenia, progeria, and frailty.

In 2008, members of the Society of Cachexia and Wasting Disorders published diagnostic criteria of non-cancer-specific cachexia, defined as 5% weight loss in the previous 6 months with at least three of five clinical symptoms: fatigue, anorexia, reduced muscle strength, reduced fat-free mass, and/or systemic signs of inflammation. In 2009, the Italian research group SCRINIO defined cancer-specific cachexia as weight loss >10% with symptoms of anorexia, early satiety, and fatigue. In 2011, an international Delphi consensus definition and classification of cancer cachexia was published, provisionally defining cancer cachexia as >5% weight loss in the previous 6 months or 2%-5% weight loss with either a body mass index (BMI) of <20 kg/m² or reduced muscle mass.

Cachexia is associated with an abnormal energy and substrate metabolism that cannot be reversed by conventional nutritional support. This differentiates the syndrome from (semi)starvation during which energy expenditure and protein turnover is reduced.

According to estimates, more than 160,000 people are hospitalized with a cachexia diagnosis every year in the United States.

Cachexia is highly prevalent in advanced cancers. One third of all subjects with cancer lose more than 5% of their original body weight, which is a common screening criterion for cancer cachexia. Particularly, about 90% of liver and pancreatic cancer subjects are at risk of developing cachexia. Cancer cachexia is a debilitating syndrome characterized by involuntary weight loss that not only affects adipose tissue but also leads to wasting and weakness of skeletal muscle. Cachexia is associated with poor clinical outcome, decreased survival, and negatively influences tumor therapy, as is illustrated by increased postoperative mortality and decreased response to radiation-, chemo-, and immunotherapy. Muscle wasting is an important contributing factor to muscle weakness in cachexia, which adversely affects performance status, quality of life and hospitalization risk of cancer subjects.

The cachexia can also be defined: (a) as having (being associated with) at least two of the symptoms selected from the group consisting of: 1) a hyper-inflammatory state, 2) altered hormone levels and/or cytokine levels; 3) decreased heart rate variability; 4) weight loss, and 5) sustained increased heart rate, wherein optionally the sustained increased heart rate is having a sustained elevated heart rate of at least about 6 bpm; or (b) by an individual having at least a sustained elevated heart rate of at least about 6 bpm and weight loss.

### Existing Treatments and Need for Improved Treatment

Treatment of cachexia will often depend on the associated underlying condition. Due to the complex nature of cachexia, simply increasing calorie consumption will often not be enough to stop weight loss and muscle degradation.

Current therapies and interventions may include dietary counseling, dietary supplements, exercise, pharmacologic agents, or multimodal approaches. Pharmacologic approaches may include appetite stimulants (cannabis and cannabinoids, corticosteroids, cyproheptadine, megestrol acetate), and anabolic agents (anamorelin, androgens, or selective androgen receptor modulators). Other pharmacological agents that have been previously employed to treat cachexia include adenosine triphosphate, insulin, mirtazapine, melatonin, nonsteroidal anti-inflammatory agents (NSAIDS), and olanzapine.

Megestrol acetate improves appetite and body weight in subjects with cancer cachexia. However, the type of weight gain associated with megestrol acetate use is primarily adipose tissue, rather than skeletal muscle. Whether efficacy can be improved further by combining megestrol acetate with other agents remains uncertain. Toxicities of megestrol acetate include thromboembolic events, edema, and adrenal suppression. Similar findings have been observed with medroxyprogesterone acetate.

Corticosteroids also improve appetite to a similar degree as seen with megestrol acetate. However, given the toxicities and decline in efficacy associated with long-term use of corticosteroids, their role as an appetite stimulant often is limited to subjects with a life expectancy of weeks to a couple months.

MAB-P1 is a monoclonal antibody targeting interleukin la that has shown promise in a placebo-controlled trial of subjects with refractory metastatic colorectal cancer and weight loss; this antibody remains investigational.

Evidence remains insufficient to strongly endorse any pharmacologic agent to improve cancer cachexia outcomes; clinicians may choose not to offer medications for the treatment of cancer cachexia. There are currently no FDA-approved medications for the indication of cancer cachexia. Therefore, there is a need for the development of effective cachexia treatments for cancer-related and other forms of cachexia.

### Description of Embodiments

Herein, we describe the use of compounds that selectively inhibit both Vascular endothelial growth factor receptor 2 (VEGFR2) and fibroblast growth factor receptor 1 (FGFR1) (i.e., a selective inhibitor of VEGFR2 and FGFR1) as treatments for lipid storage in subcutaneous adipose tissue, fat mass increase, brown fat mass increase, bone density, lean muscle mass increase, the thickening of abdominal subcutaneous adipose tissue or decreasing liver lipid content in a subject comprising administering to the subject a selective inhibitor of VEGFR2 and FGFR1, wherein the administration promotes the lipid storage or fat mass increase in the subject as compared to administration of a control agent or as compared to historical data obtained from the subject prior to the administration of the selective inhibitor of VEGFR2 and FGFR.

We also describe a method of treating, preventing, reversing, or delaying progression of a wasting syndrome in a subject comprising administering to the subject a selective inhibitor of VEGFR2 and FGFR1

In some embodiments, the selective inhibitors comprise a single agent that selectively inhibit both VEGFR2 and FGFR1.

Brivanib Alaninate and SU5402 are two compounds that each selectively inhibit VEGFR2 and FGFR1. We show that these two inhibitors were, individually, successful at preserving fat mass and bone density in a mouse model of progeria.

Cediranib maleate, a VEGFR2 inhibitor, and Infigratinib, an FGFR1 inhibitor, were, individually, not able to preserve fat mass, muscle mass, or weight in a mouse model of progeria. The combination of the administration of a selective inhibitor of VEGFR2 and a selective inhibitor of FGFR1 can recapitulate the effects of a selective inhibitor of VEGFR2 and FGFR1.

Therefore, it is the combined ability of the selective inhibitor to target VEGFR2 and FGFR1 that has allowed the successful promotion and preservation of lipid storage in subcutaneous adipose tissue, fat mass increase, brown fat mass increase, bone density, lean muscle mass increase, the thickening of abdominal subcutaneous adipose tissue or decreasing liver lipid content in the mouse model of progeria.

In some embodiments, the wasting syndrome is selected from the group consisting of lipoatrophy, cachexia, liver disease, kidney disease, fatty liver disease, Non-alcoholic Steato Hepatitis (NASH), diabetes, osteoporosis, Paget's disease of the bone, osteoarthritis, rheumatoid arthritis, sarcopenia, and frailty.

Methods of measuring subcutaneous adipose tissue, fat mass increase, brown fat mass increase, bone density, lean muscle mass increase, the thickening of abdominal subcutaneous adipose tissue or decreasing liver lipid content in a subject include the methods known in the common technical knowledge.

Methods of measuring subcutaneous adipose tissue, fat mass increase, and/or brown fat mass increase in a subject include Mill (magnetic resonance imaging), such as Echo MRI, DXA, ultrasound, bioimpedance, or calipers.

Methods of measuring bone density in a subject include x-ray scans or dual-energy X-ray absorptiometry (DEXA or DXA). H & E stained bone samples scored by a pathologist can also determine bone density.

Methods of measuring muscle mass include calipers, bioelectrical impedance scale, or by subtracting known weight of fat from the total body weight.

Methods of measuring liver lipid content in a subject include CT scans, Mill, ultrasound, magnetic resonance spectroscopy, and liver biopsy.

### Selection of Inhibitors

Compounds the selectively inhibit both VEGFR2 and FGFR1 were selected based on first identifying compounds that have reported target IC₅₀ values toward VEGFR2 and FGFR1 individually.

Those results were then filtered for compounds that have a reported IC₅₀ values to VEGFR2 and FGFR1 that are less than or equal to 10 uM. The compounds were then narrowed to compounds that have fewer than 10 reported targets. The selected compounds are listed in Table 1.

To identify compounds that selectively inhibit VEGFR2, compounds that have reported IC₅₀ values toward VEGFR2 were first identified. For those compounds, all reported IC₅₀ assay of that compound to any gene target were analyzed. To find selectivity, the ratio of the IC₅₀ toward VEGFR2 to the next lowest reported IC₅₀ to any gene target (relative IC₅₀) was determined. Relative IC₅₀ values <1 are considered as having selectivity of the compound toward VEGFR2. Relative IC₅₀ values of 1 report that the compound has equal activity inhibiting VEGFR2 and another gene target. Compounds with relative IC₅₀>1 are considered not to have selectivity to inhibiting VEGFR2. The selected compounds are listed in Table 3.

To identify compounds that selectively inhibit FGFR1, compounds that have reported IC₅₀ values toward FGFR1 were first identified. For those compounds, all reported IC₅₀ assay of that compound to any gene target were analyzed. To find selectivity, the ratio of the IC50 toward FGFR1 to the next lowest reported IC₅₀ to any gene target (relative IC₅₀) was determined. Relative IC₅₀ values <1 are considered as having selectivity of the compound toward FGFR1. Relative IC₅₀ values of 1 report that the compound has equal activity inhibiting FGFR1 and another gene target. Compounds with relative IC₅₀>1 are considered not to have selectivity to inhibiting FGFR1. The selected compounds are listed in Table 2.

In some embodiments, the selective VEGFR2 and FGFR1 inhibitor is Brivanib Alaninate.

In some embodiments, the selective VEGFR2 and FGFR1 inhibitor is SU5402.

### Pharmaceutical Formulations and Routes of Administration

The methods described herein use pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, cyclodextrins, modified cyclodextrins (i.e., sufobutyl ether cyclodextrins), etc. Suitable excipients for non-liquid formulations are also known to those of skill in the art.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid,

Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In some embodiments, the mode of administration is a solid dosage form, such as tablets and pills that are orally administered.

Pharmaceutically acceptable salts can be used in the compositions of the present embodiments and include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions may be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, may include other pharmaceutical agents, adjuvants, diluents, buffers, etc.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In therapeutic applications, the dosages of the pharmaceutical compositions used in accordance with the disclosure vary depending on the agent, the age, weight, and clinical condition of the recipient subject, and the experience and judgment of the clinician or practitioner administering the therapy, among other factors affecting the selected dosage. Generally, the dose should be sufficient to result in slowing, and preferably regressing, the symptoms of the wasting syndrome or disorder disclosed herein and optionally causing complete regression of the disease or disorder. An effective amount of a pharmaceutical agent is that which provides an objectively identifiable improvement as noted by the clinician or other qualified observer. Improvement in survival and growth indicates regression.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic or prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the subject, and the route of administration.

### Dosing Regimens

In some embodiments, a pharmaceutically or therapeutically effective amount of a selective inhibitor of VEGFR2 and FGFR1 are delivered to the subject.

In some embodiments, a pharmaceutically or therapeutically effective amount of a selective inhibitor of VEGFR2 and a selective inhibitor of FGFR1 are delivered to the subject. In these embodiments, the pharmaceutically or therapeutically effective amount of the selective inhibitor of VEGFR2 and the pharmaceutically or therapeutically effective amount of the selective inhibitor of FGFR1 each are as described in the paragraphs below, which set forth the dosing regimen for the single selective inhibitor.

In some embodiments, a pharmaceutically or therapeutically effective amount of a single selective inhibitor of VEGFR2 and FGFR1 is between about 10 mg/day and 200 mg/ day. (e.g., between about 10 mg/day and about 190 mg/day, between about 10 mg/day and about 180 mg/day, between about 10 mg/day and about 170 mg/day, between about 10 mg/day and about 160 mg/day, between about 10 mg/day and about 150 mg/day, between about 10 mg/day and about 140 mg/day, between about 10 mg/day and about 130 mg/day, between about 10 mg/day and about 120 mg/day, between about 10 mg/day and about 110 mg/day, between about 10 mg/day and about 100 mg/day, between about 10 mg/day and about 90 mg/day, between about 10 mg/day and about 80 mg/day, between about 10 mg/day and about 70 mg/day, between about 10 mg/day and about 60 mg/day, between about 10 mg/day and about 50 mg/day, between about 10 mg/day and about 40 mg/day, between about 10 mg/day and about 30 mg/day, between about 10 mg/day and about 20 mg/day).

In some embodiments, the selective VEGFR2 and FGFR1 inhibitor is selected from the group consisting of [4-(2,4-Difluoro-5-methoxycarbamoyl-phenylamino)-5-isopropyl-pyrrolo[2,1-f][1,2,4]triazin-6-yl]-carbamic acid tetrahydro-furan-2-ylmethyl ester, Brivanib Alaninate (Brivanib), 2,4-Difluoro-5-[5-isopropyl-6-(5-trifluoromethyl-[1,3,4]oxadiazol-2-yl)-pyrrolo[2,1-f][1,2,4]triazin-4-ylamino]-N-methoxy- benzamide, 5-[(1-Ethylpiperidin-4-yl)amino]-3-[1H-imidazol-2-yl(phenyl)methylidene]-1H-indol-2-one, 2,4-Difluoro-5-[5-isopropyl-6-(5-methyl-4H-[1,2,4]triazol-3-yl)-pyrrolo[2,1-f][1,2,4]triazin-4-ylamino]-N-methoxy-benzamide, 5-[6-(5-Dimethylamino-[1,3,4]oxadiazol-2-yl)-5-isopropyl-pyrrolo[2,1-f][1,2,4]triazin-4-ylamino]-2,4-difluoro-N-methoxy-benzamide, 2,4-Difluoro-5-[5-isopropyl-6-(3-methyl-[1,2,4]oxadiazol-5-yl)-pyrrolo[2,1-f][1,2,4]triazin-4-ylamino]-N-methoxy-benzamide, 3-[(3-(2-Carboxyethyl)-4-methylpyrrol-2-YL)methylene]-2-indolinone (SU5402), 2,4-Difluoro-5-[5-isopropyl-6-(5-methyl-[1,3,4]oxadiazol-2-yl)-pyrrolo[2,1-f][1,2,4]triazin-4-ylamino]-N-methoxy-benzamide, 5-[6-(5-Difluoromethyl-[1,3,4]oxadiazol-2-yl)-5-isopropyl-pyrrolo[2,1-f][1,2,4]triazin-4-ylamino]-2,4-difluoro-N-methoxy-benzamidem, 3-[Benzimidazol-2-ylidene-(3-fluorophenyl)methyl]-5-[(1-ethylpiperidin-4-yl)amino]-1H-indol-2-ol, N-(Cyclopropylmethyl)-3-(1-methylpyrazol-4-yl)-N-phenylquinoxalin-6-amine, 2,4-Difluoro-5-(5-isopropyl-6-[1,3,4]oxadiazol-2-yl-pyrrolo[2,14][1,2,4]triazin-4-ylamino)-N-methoxy-benzamide, 7-(4-Fluoroanilino)-1-[(1R,3R)-3-hydroxycyclopentyl]-3-(4-methoxyphenyl)-4H-pyrimido[4,5-d]pyrimidin-2-one, 5-[6-(5-Cyclopropyl-[1,3,4]oxadiazol-2-yl)-5-isopropyl-pyrrolo[2,1-f][1,2,4]triazin-4-ylamino]-2,4-difluoro-N-methoxy-benzamide, 6-(5-Methyl-1,3,4-oxadiazol-2-yl)-5-propan-2-yl-N-(1H-pyrrolo[2,3-b]pyridin-5-yl)pyrrolo[2,1-f][1,2,4]triazin-4-amine, N-(Cyclopropylmethyl)-N-[3-methoxy-5-(trifluoromethyl)phenyl]-3-(1-methylpyrazol-4-yl)quinoxalin-6-amine, 3-[Benzimidazol-2-ylidene-(3,5-difluorophenyl)methyl]-5-[(1-ethylpiperidin-4-yl)amino]-1H-indol-2-ol, 1,6-Naphthyridine 76, 2,4-Difluoro-5-{5-isopropyl-6-[5-(2,2,2-trifluoro-ethyl)-[1,3,4]oxadiazol-2-yl]-pyrrolo[2,1-f][1,2,4]triazin-4- ylamino}-N-methoxy-benzamide, (+)-(1R,3R)-1-(3-Hydroxy-cyclopentyl)-3-(4-methoxy-phenyl)-7-phenylamino-3,4-dihydro-1H-pyrimido[4,5-d]pyrimidin-2-one, 1,6-Naphthyridine 75, (−)-(1S,3 S)-1-(3-Hydroxy-cyclopentyl)-3-(4-methoxy-phenyl)-7-phenyl amino-3,4-dihydro-1H-pyrimido[4,5-d]pyrimidin-2-one, N-(Cyclopropylmethyl)-N-[3-[(dimethylamino)methyl]-5-methoxyphenyl]-3-(1-methylpyrazol-4-yl)quinoxalin-6-amine, N-[3-(Aminomethyl)-5-methoxyphenyl]-N-(cyclopropylmethyl)-3-(1-methylpyrazol-4-yl)quinoxalin-6-amine;hydrochloride, Chemb14573547, 1,6-Naphthyridine 77, 1,6-Naphthyridine 19, 1,6-Naphthyridine 26, N-(Cyclopropylmethyl)-N-[3-(difluoromethoxy)phenyl]-3-(1-methylpyrazol-4-yl)quinoxalin-6-amine, N′-(3,4-Difluoro-5-methoxyphenyl)-N′-[3-(1-methylpyrazol-4-yl)quinoxalin-6-yl]-N-(2,2,2-trifluoroethyl)propane-1,3-diamine;hydrochloride, 2,4-Difluoro-5-[5-isopropyl-6-(5-methanesulfonylmethyl-[1,3,4]oxadiazol-2-yl)-pyrrolo[2,1-f][1,2,4]triazin-4-ylamino]-N-methoxy-benzamide, 5-(2-Chlorophenyl)-7-fluoro-1,2-dihydro-8-methoxy-3-methylpyrazolo(3,4-b)(1,4)benzodiazepine, [3-[Cyclopropylmethyl-[3-(1-methylpyrazol-4-yl)quinoxalin-6-yl]amino]-5-methoxyphenyl]-pyrrolidin-1-ylmethanone, 5-[(1-Ethylpiperidin-4-yl)amino]-3-[(2-fluorophenyl)-(5-methyl-1H-imidazol-2-yl)methylidene]-1H-indol-2-one, 5-[6-(5-Cyclopropylmethyl-[1,3,4]oxadiazol-2-yl)-5-isopropyl-pyrrolo[2,1-f][1,2,4]triazin-4-ylamino]-2,4-difluoro-N-methoxy-benzamide, 3-[(3,5-Difluorophenyl)-(5-methyl-1H-imidazol-2-yl)methylidene]-5-[(1-ethylpiperidin-4-yl)amino]-1H-indol-2-one, 5-[(1-Ethylpiperidin-4- yl)amino]-3-[(3-fluorophenyl)-(1H-imidazol-2-yl)methylidene]-1H-indol-2-one, Indolin-2-one deriv. 9c, Methyl (3Z)-2-oxo-3-[phenyl-[4-(piperidin-1-ylmethyl)anilino]methylidene]-1H-indole-6-carboxylate, N-(3,5-Dimethoxyphenyl)-N-[3-(3-methylimidazol-4-yl)prop-2-ynyl]-3-(1-methylpyrazol-4-yl)quinoxalin-6-amine, (3z)-5-[(1-Ethylpiperidin-4-Yl)amino]-3-[(5-Methoxy-1h-Benzimidazol-2-Yl)(Phenyl)methylidene]-1,3-Dihydro-2h-Indo1-2-One, Chemb14449189, [5-Amino-1-(1H-indo1-5-yl)pyrazol-4-yl]-(1H-indo1-2-yl)methanone, 5-Isopropyl-6-(5-methyl-1,3,4-oxadiazol-2-yl)-N-(2-methyl-1H-pyrrolo[2,3-b]pyridin-5-yl)pyrrolo[2,1-f][1,2,4]triazin-4-amine, 3-[Benzimidazol-2-ylidene-(4-methylphenyl)methyl]-5-[(1-ethylpiperidin-4-yl)amino]-1H-indo1-2-ol, 5-[6-(5-Ethyl-[1,3,4]oxadiazol-2-yl)-5-isopropyl-pyrrolo[2,1-f][1,2,4]triazin-4-ylamino]-2,4-difluoro-N-methoxy-benzamide, 3-[Benzimidazol-2-ylidene(phenyl)methyl]-5-[(1-ethylpiperidin-4-yl)amino]-1H-indol-2-ol, 1,6-Naphthyridine 25, 2-(6-Fluoro-1H-indazol-3-yl)-5-(4-(piperidin-1-yl)piperidin-1-yl)-1H-benzo[d]imidazole, 3-[Benzimidazol-2-ylidene-(4-methoxyphenyl)methyl]-5-[(1-ethylpiperidin-4-yl)amino]-1H-indo1-2-ol, N-(Cyclopropylmethyl)-N-[3-methoxy-5-[2-(methylamino)ethoxy]phenyl]-3-(1-methylpyrazol-4-yl)quinoxalin-6-amine, 3-Methoxy-N-methyl-5-[[3-(1-methylpyrazol-4-yl)quinoxalin-6-yl]-[2-(propan-2-ylamino)ethyl]amino]benzamide, XL999;XL-999; XL 999, 3-N-(Cyclopropylmethyl)-1-N-methyl-3-N-[3-(1-methylpyrazol-4-yl)quinoxalin-6-yl]benzene-1,3-diamine, 2,4-Difluoro-5-[6-(5-isobutyl-[1,3,4]oxadiazol-2-yl)-5-isopropyl-pyrrolo[2,1-f][1,2,4]triazin-4-ylamino]-N-methoxy-benzamide, Methyl 2-hydroxy-3-[N-[4-[2-methyl-5-[(4-methylpiperazin-1-yl)methyl]pyrrol-1-yl]phenyl]-C-phenylcarbonimidoyl]-1H-indole-6-carboxylate, N-(3,5-Dimethoxyphenyl)-3-(1-methylpyrazol-4-yl)-N-[(2-pyridin-4-ylcyclopropyl)methyl]quinoxalin-6-amine, 3-(3,5-Dimethoxyphenyl)-7-N-[3-(4-methylpiperazin-1-yl)propyl]-1,6-naphthyridine-2,7-diamine, 9-Chloro-2-[3-[3-(dimethylamino)propyl]anilino]-5,7-dihydropyrimido[5,4-d][1]benzazepine-6-thione, Pyrido[2,3-d]pyrimidine 103, Pyrido[2,3-d]pyrimidine 105, 2,4-Difluoro-5-[5-isopropyl-6-(5-methyl-oxazol-2-yl)-pyrrolo[2,1-f][1,2,4]triazin-4-ylamino]-N-methoxy-benzamide, N-(3,5-Dimethoxyphenyl)-N-[3-(2,5-dimethylimidazol-1-yl)propyl]-3-(1-methylpyrazol-4-yl)quinoxalin-6-amine;hydrochloride, N-[3-[2-[6-(2-Chlorophenyl)-2-[4-(diethylamino)butylamino]-7-oxopyrido[2,3-d]pyrimidin-8-yl]ethyl]phenyl]prop-2-enamide, N-(3,5-Dimethoxyphenyl)-3-(1-methylpyrazol-4-yl)-N-(2-piperazin-1-ylethyl)quinoxalin-6-amine;hydrochloride, Methyl (3 Z)-3-[[4-[2-(dimethylamino)ethyl-methyl sulfonylamino] anilino]-phenylmethylidene]-2-oxo-1H-indole-6-carboxylate, 4-[3-(3,5-Dimethoxy-N-[3-(1-methylpyrazol-4-yl)quinoxalin-6-yl]anilino)prop-1-ynyl]pyridine-3-carbonitrile, N-[(5-Chlorothiophen-2-yl)methyl]-N-(3,5-dimethoxyphenyl)-3-[1-(2-piperazin-1-ylethyl)pyrazol-4-yl]quinoxalin-6-amine;hydrochloride, N′-(3,5-Dichlorophenyl)-N′-[3-(1-methylpyrazol-4-yl)quinoxalin-6-yl]-N-(2,2,2-trifluoroethyl)propane-1,3-diamine, 2-[4-[7-[N-(Cyclopropylmethyl)anilino]quinoxalin-2-yl]pyrazol-1-yl]ethanol, Pyrido[2,3-d]pyrimidine 102, 7-N-[4-(Diethylamino)butyl]-3-(3,5-dimethoxyphenyl)-1,6-naphthyridine-2,7-diamine, N-(3,5-Dimethoxyphenyl)-N-[3-(4-methoxypyrimidin-2-yl)propyl]-3-(1-methylpyrazol-4-yl)quinoxalin-6-amine, Methyl 3-[[4-[(dimethylamino)methyl]anilino]-phenylmethylidene]-2-oxo-1H-indole-6-carboxylate, 3-Fluoro-N-methyl-5-[[3-(1-methylpyrazol-4-yl)quinoxalin-6-yl]-[2-(propan-2-ylamino)ethyl]amino]benzamide, N-(Cyclopropylmethyl)-N-[3-(1,3-dioxolan-2-yl)-5-methoxyphenyl]-3-(1-methylpyrazol-4-yl)quinoxalin-6-amine, N-[[(2S)-1-[3-(3,5-Dimethoxy-N-[3-(1-methylpyrazol-4-yl)quinoxalin-6-yl]anilino)propyl]pyrrolidin-2-yl]methyl]-1,1,1-trifluoromethanesulfonamide, 3-Methoxy-N-methyl-5-[2-(propan-2-ylamino)ethyl-[3-(1-propan-2-ylpyrazol-4-yl)quinoxalin-6-yl]amino]benzamide, 5-{6-[5-(Difluoro-methanesulfonyl-methyl)-[1,3,4]oxadiazol-2-yl]-5-isopropyl-pyrrolo[2,1-f][1,2,4]triazin-4-ylamino}-2,4-difluoro-N-methoxy- benzamide, N-[2-(3-Aminopiperidin-1-yl)ethyl]-N-(3,5-dimethoxyphenyl)-3-(1-methylpyrazol-4-yl)quinoxalin-6-amine, N-(3,5-Dimethoxyphenyl)-N-[(E)-3-(4-methoxypyrimidin-2-yl)prop-2-enyl]-3-(1-methylpyrazol-4-yl)quinoxalin-6-amine, Methyl (3Z)-3-[[4-[acetyl-[2-(dimethylamino)ethyl]amino]anilino]-phenylmethylidene]-2-oxo-1H-indole-6-carboxylate, Methyl N-[4-[7-amino-3-(3,4-dimethoxyphenyl)-5-(1-methylpiperidin-4-yl)oxypyrazolo[1,5-a]pyrimidin-6-yl]phenyl]carbamate, Anilinoquinazoline deriv. 35, 1,6-Naphthyridine 17, Tert-butyl (1S,4S)-5-[2-(3,5-dimethoxy-N-[3-(1-methylpyrazol-4-yl)quinoxalin-6-yl]anilino)ethyl]-2,5-diazabicyclo[2.2.1]heptane-2- carboxylate, Lucitanib, 6-((7-((1-Aminocyclopropyl)methoxy)-6-methoxyquinolin-4-yl)oxy)-N-methyl-1-naphthamide hydrochloride, Pyrido[2,3-d]pyrimidine 104, Nintedanib esylate, [(2S)-1-(3,5-Dimethoxy-N-[3-(1-methylpyrazol-4-yl)quinoxalin-6-yl]anilino)-3-methoxypropan-2-yl] acetate, 3-[2-Aminoethyl-[3-(1-methylpyrazol-4-yl)quinoxalin-6-yl]amino]-5-methoxy-N-methylbenzamide, N-(3,5-Dimethoxyphenyl)-3-(1-methylpyrazol-4-yl)-N-[3-[4-(trifluoromethyl)piperidin-1-yl]propyl]quinoxalin-6-amine, 3-((4-Bromo-2,6-difluorobenzyl)oxy)-5-(3-(4-(pyrrolidin-1-yl)butyl)ureido)isothiazole-4-carboxamide, N-(3,5-Dimethoxyphenyl)-N-[3-(2-methylimidazol-1-yl)propyl]-3-(1-methylpyrazol-4-yl)quinoxalin-6-amine, 3-[3-(Morpholinomethyl)-4,5,6,7-tetrahydro-1H-indole-2-ylmethylene]-5-(ethylsulfonyl)-2,3-dihydro-1H-indole-2-one, 2-(3-Methoxy-N-[3-(1-methylpyrazol-4-yl)quinoxalin-6-yl]anilino)ethanol, Epihematoxylol, N-[3-(5-Aminopyrazin-2-yl)prop-2-ynyl]-N-(3,5-dimethoxyphenyl)-3-(1-methylpyrazol-4-yl)quinoxalin-6-amine, 2-[3-[2-Aminoethyl-[7-(1-methylpyrazol-4-yl)quinoxalin-2-yl]amino]-5-fluorophenoxy]ethanol, N-[3-(3-Aminopyridin-2-yl)prop-2-ynyl]-N-(3,5-dimethoxyphenyl)-3-(1-methylpyrazol-4-yl)quinoxalin-6-amine, N-(3,5-Dimethoxyphenyl)-N-[3-(3-methoxypyridin-2-yl)propyl]-3-(1-methylpyrazol-4-yl)quinoxalin-6-amine, N-(3,5-Dimethoxyphenyl)-3-(1-methylpyrazol-4-yl)-N-[(Z)-3-pyrimidin-2-ylprop-2-enyl]quinoxalin-6-amine, N′-(3,5-Dimethoxyphenyl)-N-methyl-N′3-[1-(oxan-4-ylmethyl)pyrazol-4-yl]quinoxalin-6-yl]ethane-1,2-diamine;hydrochloride, [5-Amino-1-(2-chloro-5-hydroxyphenyl)pyrazol-4-yl]-[5-(morpholin-4-ylmethyl)-1H-indol-2-yl]methanone, 1,6-Naphthyridine 18, 1-Tert-butyl-3-[3-(3,5-dimethoxyphenyl)-7-[4-(4-methylpiperazin-1-yl)butylamino]-1,6-naphthyridin-2-yl]urea, N′-(2-Chloro-3,5-dimethoxyphenyl)-N′-[3-(1-methylpyrazol-4-yl)quinoxalin-6-yl]-N-propan-2-ylethane-1,2-diamine;hydrochloride, N-(3,5-Dimethoxyphenyl)-3-(1-methylpyrazol-4-yl)-N-(3-piperazin-1-ylpropyl)quinoxalin-6-amine, Methyl (3Z)-3-[[4-[acetyl-[3-(dimethylamino)propyl]amino]anilino]-phenylmethylidene]-2-oxo-1H-indole-6-carboxylate, 3-[4-[3-[4-[7-[N-(Cyclopropylmethyl)-3,5-dimethoxyanilino]quinoxalin-2-yl]pyrazol-1-yl]propyl]piperazin-1-yl]propan-1-ol;hydrochloride, 5-[(R)-1-(3,5-Dichloropyridine-4-yl)ethoxy]-3-[5-(4-methylpiperazine-1-yl)-1H-benzoimidazole-2-yl]-1H-indazole, 4-Amino-3-(1H-benzo[d]imidazol-2-yl)quinolin-2(1H)-one, 3-[4-[3-[4-[7-[3,5-Dimethoxy-N-[2-(propan-2-ylamino)ethyl]anilino]quinoxalin-2-yl]pyrazol-1-yl]propyl]piperazin-1-yl]propan-1- ol;hydrochloride, 2-(2,6-Difluoro-3,5-dimethoxy-N-[3-(1-methylpyrazol-4-yl)quinoxalin-6-yl]anilino)ethanol, [4-[7-[3,5-Dimethoxy-N-[2-(propan-2-ylamino)ethyl]anilino]quinoxalin-2-yl]-2-methylpyrazol-3-yl]methanol, 1,6-Naphthyridine deriv. 20, N-(2-Chloro-3,5-dimethoxyphenyl)-3-(1-methylpyrazol-4-yl)-N-(3-pyrrolidin-1-ylpropyl)quinoxalin-6-amine;hydrochloride, Methyl 3-[N-[4-[2-(dimethylamino)ethyl-methylcarbamoyl]phenyl]-C-phenylcarbonimidoyl]-2-hydroxy-1H-indole-6-carboxylate, N-[2-(1,4-Diazepan-l-yl)ethyl]-N-(3,5-dimethoxyphenyl)-3-(1-methylpyrazol-4-yl)quinoxalin-6-amine;oxalic acid, N′-(2,6-Difluoro-3,5-dimethoxyphenyl)-N′-[3-(1-methylpyrazol-4-yl)quinoxalin-6-yl]-N-propan-2-ylethane-1,2-diamine, N-(3,5-Dimethoxyphenyl)-N-[(3-methyloxetan-3-yl)methyl]-3-(1-methylpyrazol-4-yl)quinoxalin-6-amine, Methyl (3Z)-3-[[4-[acetyl-[2-(dimethylamino)-2-oxoethyl]amino]anilino]-phenylmethylidene]-2-oxo-1H-indole-6-carboxylate, Chemb14213341, Anilinoquinazoline deriv. 36, [5-Amino-1-(1H-benzimidazol-6-yl)-1H-pyrazol-4-yl]-1H-indo1-2-ylMethanone, Hematoxylone, N-Cyclopropyl-N′-(3,5-dimethoxyphenyl)-N′-[3-(1-methylpyrazol-4-yl)quinoxalin-6-yl]ethane-1,2-diamine, N-(3,5-Dimethoxyphenyl)-3-(1-methylpyrazol-4-yl)-N-(3-pyrimidin-2-ylpropyl)quinoxalin-6-amine, N′-(3,5-Dimethoxyphenyl)-N′-[3-[5-[(dimethylamino)methyl]-1-methylpyrazol-4-yl]quinoxalin-6-yl]-N-propan-2-ylethane-1,2-diamine, N′-(2,6-Difluoro-3,5-dimethoxyphenyl)-N′-[3-(1-methylpyrazol-4-yl)quinoxalin-6-yl]-N-(2,2,2-trifluoroethyl)propane-1,3-diamine, [5-Amino-1-(2-methyl-1H-indo1-5-yl)pyrazol-4-yl]-(1H-indol-2-yl)methanone, Haematoxylin, N′-(3,5-Dimethoxyphenyl)-N′-[3-(5-ethyl-1-methylpyrazol-4-yl)quinoxalin-6-yl]-N-(2,2,2-trifluoroethyl)ethane-1,2-diamine, Methyl 3-[N-[4-[[dimethylcarbamoyl(methyl)amino]methyl]phenyl]-C-phenylcarbonimidoyl]-2-hydroxy-1H-indole-6-carboxylate, 3-Methoxy-N-methyl-5-[[3-(1-methylpyrazol-4-yl)quinoxalin-6-yl]-(3-pyrrolidin-1-ylpropyl)amino]benzamide, N-(3,5-Dimethoxyphenyl)-3-(1-methylpyrazol-4-yl)-N-(3-pyridin-4-ylpropyl)quinoxalin-6-amine, 1-[3-(3,5-Dimethoxy-N-[3-(1-propan-2-ylpyrazol-4-yl)quinoxalin-6-yl]anilino)propyl]pyrrolidin-2-one, and Regorafenib.

In some embodiments, the selective inhibitor of VEGFR2 and FGFR1 is administered 6 times/day (e.g. every 4 hours).

In some embodiments, the method comprises administering to the subject 6 times/day (e.g., every 4 hours) a dose between about 10 mg/day and 200 mg/day. (e.g., between about 10 mg/day and about 190 mg/day, between about 10 mg/day and about 180 mg/day, between about 10 mg/day and about 170 mg/day, between about 10 mg/day and about 160 mg/day, between about 10 mg/day and about 150 mg/day, between about 10 mg/day and about 140 mg/day, between about 10 mg/day and about 130 mg/day, between about 10 mg/day and about 120 mg/day, between about 10 mg/day and about 110 mg/day, between about 10 mg/day and about 100 mg/day, between about 10 mg/day and about 90 mg/day, between about 10 mg/day and about 80 mg/day, between about 10 mg/day and about 70 mg/day, between about 10 mg/day and about 60 mg/day, between about 10 mg/day and about 50 mg/day, between about 10 mg/day and about 40 mg/day, between about 10 mg/day and about 30 mg/day, between about 10 mg/day and about 20 mg/day).

In some embodiments, the selective inhibitor of VEGFR2 and FGFR1 is administered 4 times/day (e.g. every 6 hours).

In some embodiments, the method comprises administering to the subject 4 times/day (e.g., every 6 hours) a dose between about 10 mg/day and 200 mg/day. (e.g., between about 10 mg/day and about 190 mg/day, between about 10 mg/day and about 180 mg/day, between about 10 mg/day and about 170 mg/day, between about 10 mg/day and about 160 mg/day, between about 10 mg/day and about 150 mg/day, between about 10 mg/day and about 140 mg/day, between about 10 mg/day and about 130 mg/day, between about 10 mg/day and about 120 mg/day, between about 10 mg/day and about 110 mg/day, between about 10 mg/day and about 100 mg/day, between about 10 mg/day and about 90 mg/day, between about 10 mg/day and about 80 mg/day, between about 10 mg/day and about 70 mg/day, between about 10 mg/day and about 60 mg/day, between about 10 mg/day and about 50 mg/day, between about 10 mg/day and about 40 mg/day, between about 10 mg/day and about 30 mg/day, between about 10 mg/day and about 20 mg/day).

In some embodiments, the selective inhibitor of VEGFR2 and FGFR1 is administered 3 times/day (e.g. every 8 hours).

In some embodiments, the method comprises administering to the subject 3 times/day (e.g., every 8 hours) a dose between about 10 mg/day and 200 mg/ day. (e.g., between about 10 mg/day and about 190 mg/day, between about 10 mg/day and about 180 mg/day, between about 10 mg/day and about 170 mg/day, between about 10 mg/day and about 160 mg/day, between about 10 mg/day and about 150 mg/day, between about 10 mg/day and about 140 mg/day, between about 10 mg/day and about 130 mg/day, between about 10 mg/day and about 120 mg/day, between about 10 mg/day and about 110 mg/day, between about 10 mg/day and about 100 mg/day, between about 10 mg/day and about 90 mg/day, between about 10 mg/day and about 80 mg/day, between about 10 mg/day and about 70 mg/day, between about 10 mg/day and about 60 mg/day, between about 10 mg/day and about 50 mg/day, between about 10 mg/day and about 40 mg/day, between about 10 mg/day and about 30 mg/day, between about 10 mg/day and about 20 mg/day).

In some embodiments, the selective inhibitor of VEGFR2 and FGFR1 is administered 2 times/day (e.g. every 12 hours).

In some embodiments, the method comprises administering to the subject 2 times/day (e.g., every 12 hours) a dose between about 10 mg/day and 200 mg/ day. (e.g., between about 10 mg/day and about 190 mg/day, between about 10 mg/day and about 180 mg/day, between about 10 mg/day and about 170 mg/day, between about 10 mg/day and about 160 mg/day, between about 10 mg/day and about 150 mg/day, between about 10 mg/day and about 140 mg/day, between about 10 mg/day and about 130 mg/day, between about 10 mg/day and about 120 mg/day, between about 10 mg/day and about 110 mg/day, between about 10 mg/day and about 100 mg/day, between about 10 mg/day and about 90 mg/day, between about 10 mg/day and about 80 mg/day, between about 10 mg/day and about 70 mg/day, between about 10 mg/day and about 60 mg/day, between about 10 mg/day and about 50 mg/day, between about 10 mg/day and about 40 mg/day, between about 10 mg/day and about 30 mg/day, between about 10 mg/day and about 20 mg/day).

In some embodiments, the selective inhibitor of VEGFR2 and FGFR1 is administered 1 time/day (e.g. every 24 hours).

In some embodiments, the method comprises administering to the subject 1 time/day (e.g., every 24 hours) a dose between about 10 mg/day and 200 mg/day. (e.g., between about 10 mg/day and about 190 mg/day, between about 10 mg/day and about 180 mg/day, between about 10 mg/day and about 170 mg/day, between about 10 mg/day and about 160 mg/day, between about 10 mg/day and about 150 mg/day, between about 10 mg/day and about 140 mg/day, between about 10 mg/day and about 130 mg/day, between about 10 mg/day and about 120 mg/day, between about 10 mg/day and about 110 mg/day, between about 10 mg/day and about 100 mg/day, between about 10 mg/day and about 90 mg/day, between about 10 mg/day and about 80 mg/day, between about 10 mg/day and about 70 mg/day, between about 10 mg/day and about 60 mg/day, between about 10 mg/day and about 50 mg/day, between about 10 mg/day and about 40 mg/day, between about 10 mg/day and about 30 mg/day, between about 10 mg/day and about 20 mg/day).

In some embodiments, the selective inhibitor of VEGFR2 and FGFR1 is Brivanib alaninate (Brivanib). In some embodiments, Brivanib alaninate is administered to a subject at a dose between 10 mg/kg-200 mg/kg per day. In some embodiments, Brivanib alaninate is administered to a subject at a dose of about 160 mg/day. In some embodiments, Brivanib alaninate is administered to a subject at a dose of about 150 mg/day. In some embodiments, Brivanib alaninate is administered to a subject at a dose of about 140 mg/day. In some embodiments, Brivanib alaninate is administered to a subject at a dose of about 130 mg/day. In some embodiments, Brivanib alaninate is administered to a subject at a dose of about 120 mg/day. In some embodiments, Brivanib alaninate is administered to a subject at a dose of about 110 mg/day. In some embodiments, Brivanib alaninate is administered to a subject at a dose of about 100 mg/day. In some embodiments, Brivanib alaninate is administered to a subject at a dose of about 90 mg/day. In some embodiments, Brivanib alaninate is administered to a subject at a dose of about 80 mg/day. In some embodiments, Brivanib alaninate is administered to a subject at a dose of about 70 mg/day. In some embodiments, Brivanib alaninate is administered to a subject at a dose of about 60 mg/day. In some embodiments, Brivanib alaninate is administered to a subject at a dose of about 50 mg/day. In some embodiments, Brivanib alaninate is administered to a subject at a dose of about 40 mg/day. In some embodiments, Brivanib alaninate is administered to a subject at a dose of about 30 mg/day. In some embodiments, Brivanib alaninate is administered to a subject at a dose of about 20 mg/day. In some embodiments, Brivanib alaninate is administered to a subject at a dose of about 10 mg/day.

In some embodiments, the selective inhibitor of VEGFR2 and FGFR1 is SU5402 In some embodiments, SU5402 is administered to a subject at a dose between 10 mg/kg-200 mg/kg per day. In some embodiments, SU5402 is administered to a subject at a dose of about 160 mg/day. In some embodiments, SU5402 is administered to a subject at a dose of about 150 mg/day. In some embodiments, SU5402 is administered to a subject at a dose of about 140 mg/day. In some embodiments, SU5402 is administered to a subject at a dose of about 130 mg/day. In some embodiments, SU5402 is administered to a subject at a dose of about 120 mg/day. In some embodiments, SU5402 is administered to a subject at a dose of about 110 mg/day. In some embodiments, SU5402 is administered to a subject at a dose of about 100 mg/day. In some embodiments, SU5402 is administered to a subject at a dose of about 90 mg/day. In some embodiments, SU5402 is administered to a subject at a dose of about 80 mg/day. In some embodiments, SU5402 is administered to a subject at a dose of about 70 mg/day. In some embodiments, SU5402 is administered to a subject at a dose of about 60 mg/day. In some embodiments, SU5402 is administered to a subject at a dose of about 50 mg/day. In some embodiments, SU5402 is administered to a subject at a dose of about 40 mg/day. In some embodiments, SU5402 is administered to a subject at a dose of about 30 mg/day. In some embodiments, SU5402 is administered to a subject at a dose of about 20 mg/day. In some embodiments, SU5402 is administered to a subject at a dose of about 10 mg/day.

EXAMPLES

### Example 1. Brivanib Alaninate preserves fat mass in a Mouse Model of Progeria

Zmpste24 mice, a progeria model mouse line, were dosed with either 50 mg/kg Brivanib alaninate for 56 days, 5 days on, 2 days off (QD5), or vehicle alone. The dose was administered via oral gavage as a suspension of 18% (w/v) PEG400 2% (w/v) Tween-80 in water . The vehicle alone was 18% (w/v) PEG400 2% (w/v) Tween-80 in water.

The body fat of the mice was measured by an Echo MRI throughout the treatment.

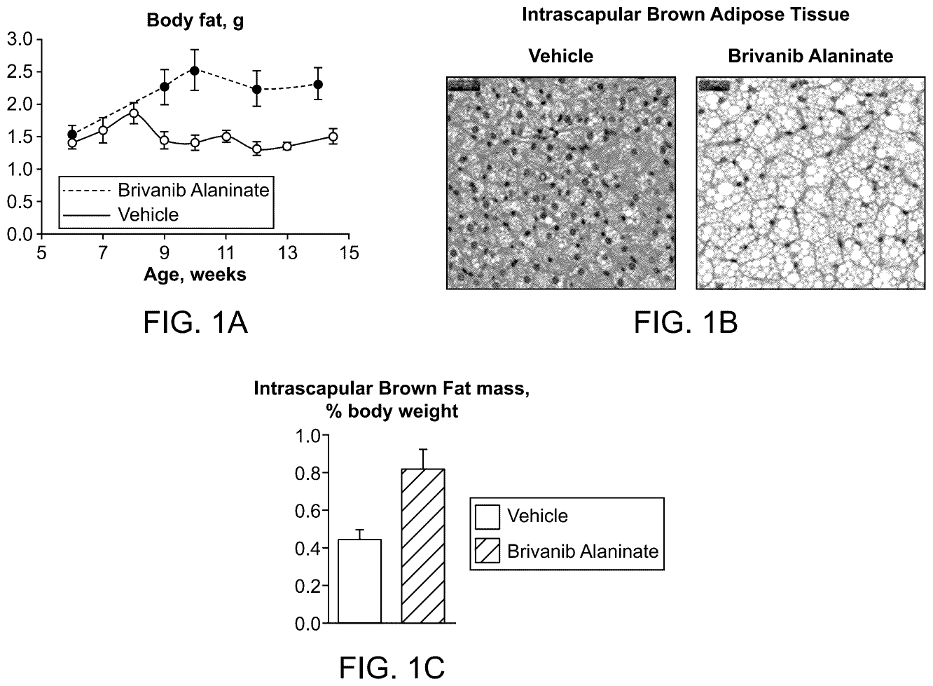

The mice that were treated with Brivanib alaninate showed an increase in body mass starting at 6 weeks of age (FIG. 1A).

Samples of intrascapular brown adipose tissue from 15 week old mice treated with Brivanib Alaninate or vehicle for 56 days underwent Hematoxylin and Eosin (H&E) staining. The tissue samples showed that Brivanib Alaninate treatment increased lipid content in intrascapular brown adipose tissue (FIG. 1B).

Intrascapular brown fat mass also made up for more of the percent of the body weight of the 15-week old male mice treated with Brivanib Alaninate versus the vehicle (FIG. 1C).

The data show successful use of Brivanib Alaninate for increasing fat mass in a mouse model of progeria.

### Example 2. Brivanib Alaninate Preserves Bone Density in a Mouse Model of Progeria

Zmpste24−/− mice were dosed with either 50 mg/kg Brivanib Alaninate for 56 days, 5 days on, 2 days off (QD5), or vehicle alone. The dose was administered via oral gavage as a suspension of 18% (w/v) PEG400 2% Tween-80 (w/v) in water . The vehicle alone was 18% (w/v) PEG400 2% (w/v) Tween-80 in water.

The mice were assessed for bone loss in the vertebrae and femur at 15 weeks of age. Bone loss was scored by a pathologist using H&E stained sections of vertebrae or femur samples by the following criteria:

Bone loss score: = ~percentage loss
of the total area of bone

0 = none
0.5 = less than 1%
1 = 1-10 %
2 = 11-25%
3 = 26-50%
4 = 51-75%
5 = greater than 75%

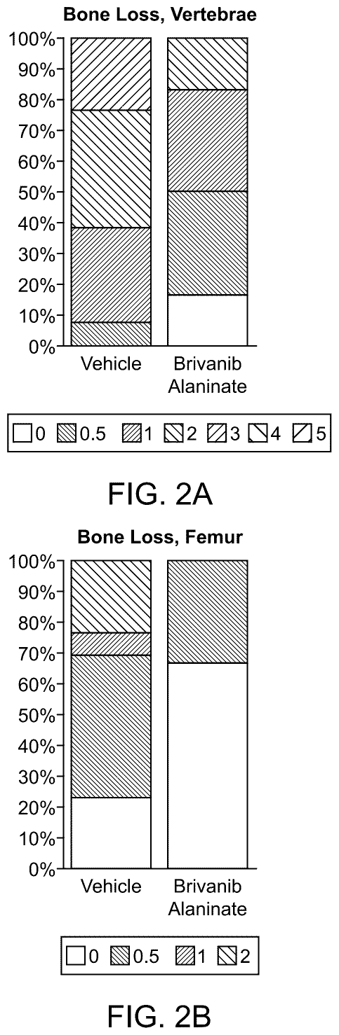

Mice treated with Brivanib Alaninate had lower bone loss scores compared to mice treated with the vehicle. For the vertebrae samples, the most severe scored assigned to the Brivanib Alaninate group was a score 4, of which compose about 15% of the mice. For the vehicle group, 20% had a score of 5 (FIG. 2A) For the femur samples, the most severe scored assigned to the Brivanib Alaninate group was a score of 0.5, which composed about 30% of the group. For the vehicle group, 20% had a score of 5 (FIG. 2B). The data show successful use of Brivanib Alaninate for the preservation of bone density.

### Example 3. Brivanib Alaninate Decreases Liver Lipid Content and Increases Storage in Subcutaneous Depot in a Mouse Model of Diet Induced Fatty Liver Disease

26 week old male C57BL6 mice were fed a high fat, high cholesterol, high fructose diet for 23 weeks. The diet provided 40 Kcal% fat from palm oil, 20% kcal% fructose and 2% cholesterol. The mice were then either treated with 25 mg/kg Brivanib Alaninate, administered in 10% (w/v) DMSO, 4% (w/v) Tween-80, 86% (w/v) PBS, or the vehicle alone, 10% (w/v) DMSO, 4% (w/v) Tween-80, 86% PBS. The mice were dosed for 28 days, once a day, via intraperitoneal injection

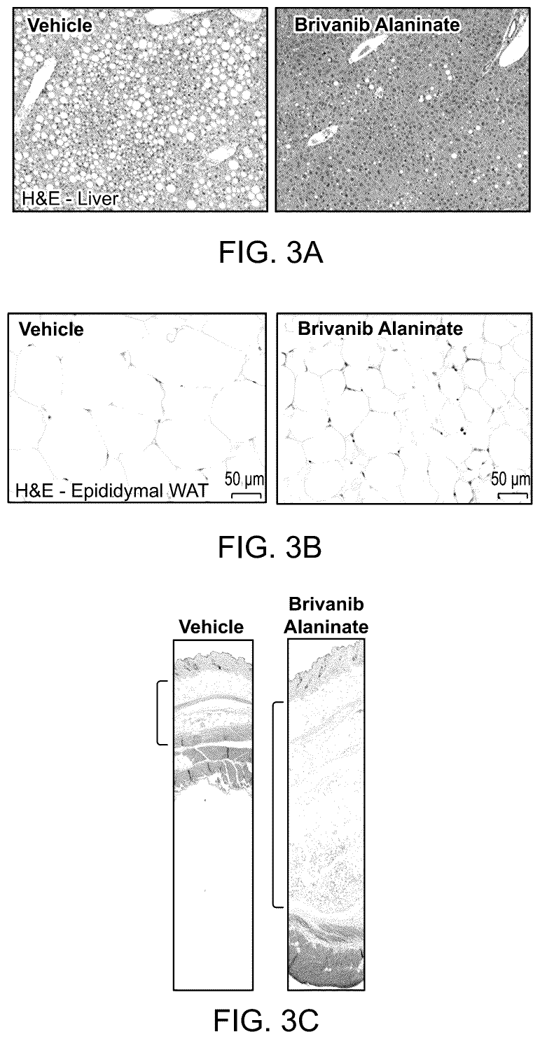

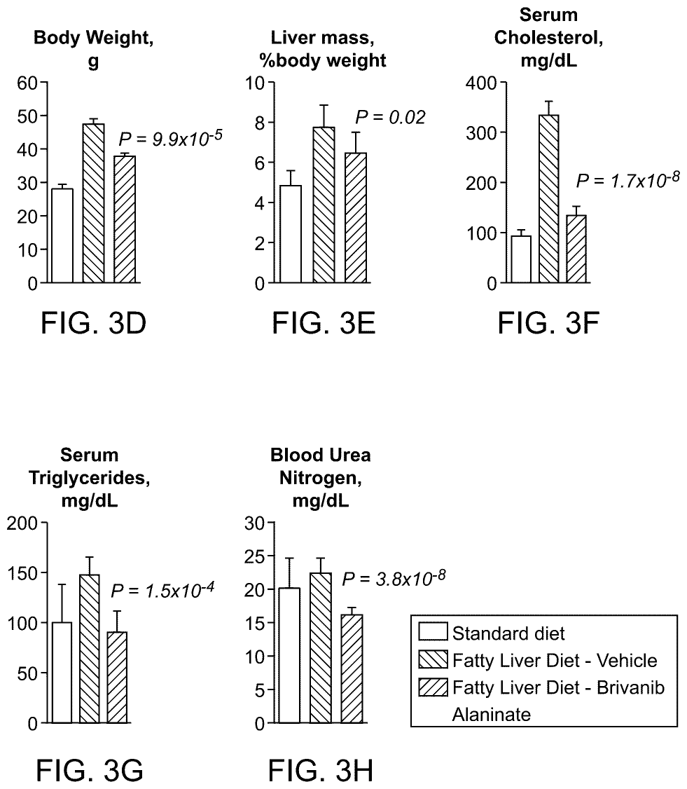

At the end of treatment, H&E stained liver samples showed Brivanib Alaninate treatment decreased liver lipid content (FIG. 3A) and decreased the size of adipocytes in the epididymal fat pad (FIG. 3B) compared to the vehicle. Brivanib alaninate treatment also caused an increased in the thickness of the abdominal subcutaneous adipose (FIG. 3C).

The data show that Brivanib Alaninate treatment increased lipid storage in subcutaneous depot in a mouse model of diet induced fatty liver disease.

26 week old male C57BL6 mice were fed a high fat, high cholesterol, high fructose diet for 23 weeks. The diet provided 40 Kcal % fat from palm oil, 20% kcal % fructose and 2% cholesterol. The mice were then either treated with 50 mg/kg Brivanib Alaninate administered in 10% (w/v) DMSO, 4% (w/v) Tween-80, 86% (w/v) PBS, or the vehicle alone, 10% (w/v) DMSO, 4% (w/v) Tween-80, 86% PBS. The mice were dosed for 28 days, once a day, via intraperitoneal injection. A third group of mice were fed a standard diet and administered the vehicle alone.

The group fed with the fatty liver diet and treated with Brivanib Alaninate had less of an increase in body weight, liver mass, serum cholesterol, serum triglycerides, and blood urea nitrogen compared to the mice fed with the fatty liver diet and treated with vehicle compared to the mice fed a standard diet (FIG. 3D-FIG. 3H). This shows the beneficial effect of Brivanib Alaninate directing the fat increase to the abdominal subcutaneous adipose and not the liver.

### Example 4. SU5402 Increase Adipose, Fat, and Body Mass of Progeria Animals

Zmpste24−/− mice, a progeria model mouse line, were dosed with either 25 mg/kg SU5402 for 56 days, 5 days on, 2 days off (QD5) or vehicle alone. The dose was administered via oral gavage as a suspension of 5% (w/v) DMSO 9% (w/v) PEG400 1% (w/v) Tween-80 in saline. The vehicle alone was 5% (w/v) DMSO 9% (w/v) PEG400 1% (w/v) Tween-80 in saline.

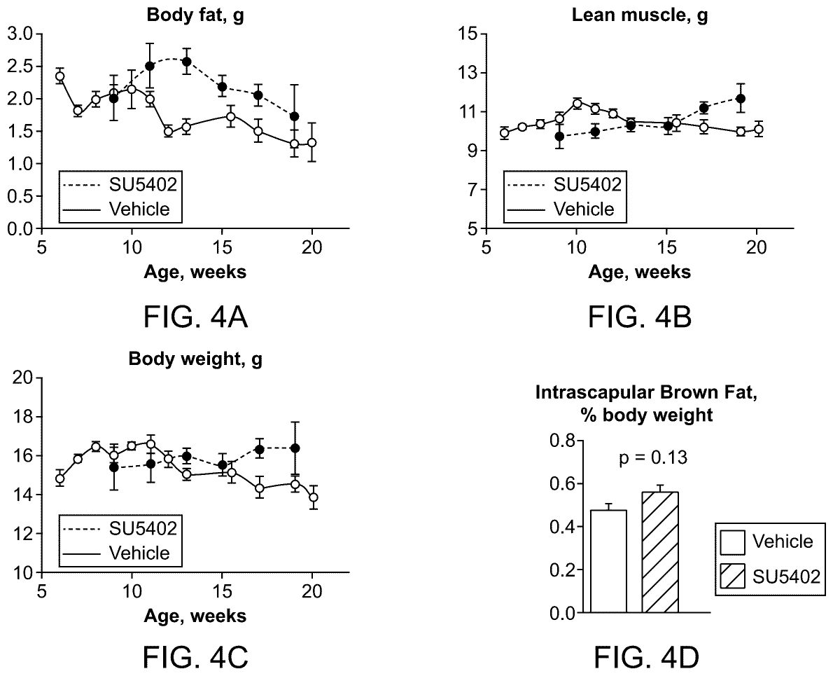

The body fat of the mice was measured by an Echo MM. Lean muscle and body weight were also measured. The mice that were treated with SU5402 had an increase in body mass starting at 9 weeks of age compared to the vehicle group (FIG. 4A). The SU5402 treated mice also had an increase in lean muscle starting at 15 weeks of age (FIG. 4B), and an increase in body weight starting at 12 weeks of age (FIG. 4C).

Intrascapular brown fat mass also made up for more of the body weight percent of the SU5402 treated female mice compared to vehicle starting at 9 weeks of age (FIG. 4D).

The data show successful use of SU5402 to increase body fat, body mass, and brown fat mass in a mouse model of progeria.

### Example 5. Cediranib Maleate, Which Targets VEGF-Receptors, Does Not Increase (Brown) Fat, Muscle or Body Mass in Progeria Model

Cediranib maleate was developed to inhibit VEGF-receptor kinases. It inhibits VEGFR1 with an IC₅₀ of 5 nM, VEGFR2 with an IC₅₀ of 1 nM, and VEGFR3 with an IC₅₀ of 3 nM.

Zmpste24−/− mice were dosed with either 5 mg/kg Cediranib maleate for 56 days, 5 days on, 2 days off (QD5) or vehicle alone. The dose was administered via oral gavage as a suspension of 18% (w/v) PEG400 2% (w/v) Tween-80 in water . The vehicle alone was 18% (w/v) PEG400 2% (w/v) Tween-80 in water.

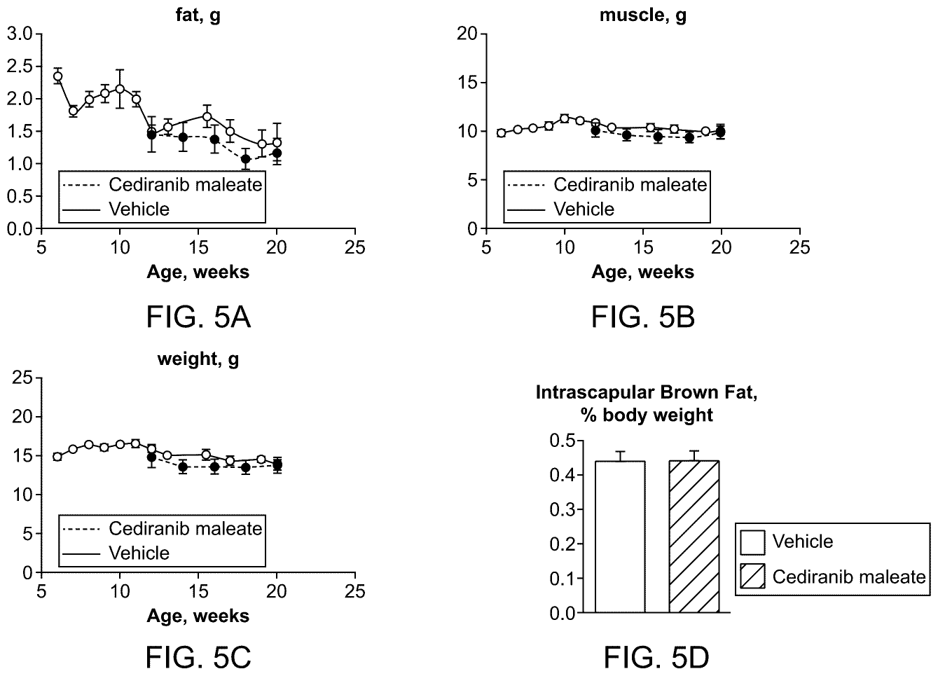

The body fat of the mice was measured by an Echo MRI. The mice treated with Cediranib maleate did not show an increase in body fat, lean muscle, or weight compared to the control group (FIG. 5A-FIG. 5C). There was also no alteration of intrascapular brown adipose tissue mass (FIG. 5D).

The data show that although Cediranib maleate inhibits the VEGF receptor family with great affinity, the compound is not able to promote an increase in fat, muscle, or weight in a mice model of progeria.

### Example 6. Regorafenib, which Targets VEGFRs, FGFR1, and Other RTKs, Does Not Increase (Brown) Fat, Muscle or Body Mass in Progeria Model

Regorafenib (Stivarga) is used as a multi-kinase inhibitor to block angiogenesis. It inhibits VEGFR1, VEGFR2, VEGFR3, EPHX2, FGFR1, RAF1, and PDGFRB with an IC₅₀ of less than 3 nM.

Zmpste24−/− mice were dosed with either 10 mg/kg Regorafenib for 56 days, 5 days on, 2 days off (QD5) or a vehicle alone. The dose was administered via oral gavage as a suspension of 18% (w/v) PEG400 2% (w/v) Tween-80 in water. The vehicle alone was 18% (w/v) PEG400 2% (w/v) Tween-80 in water.

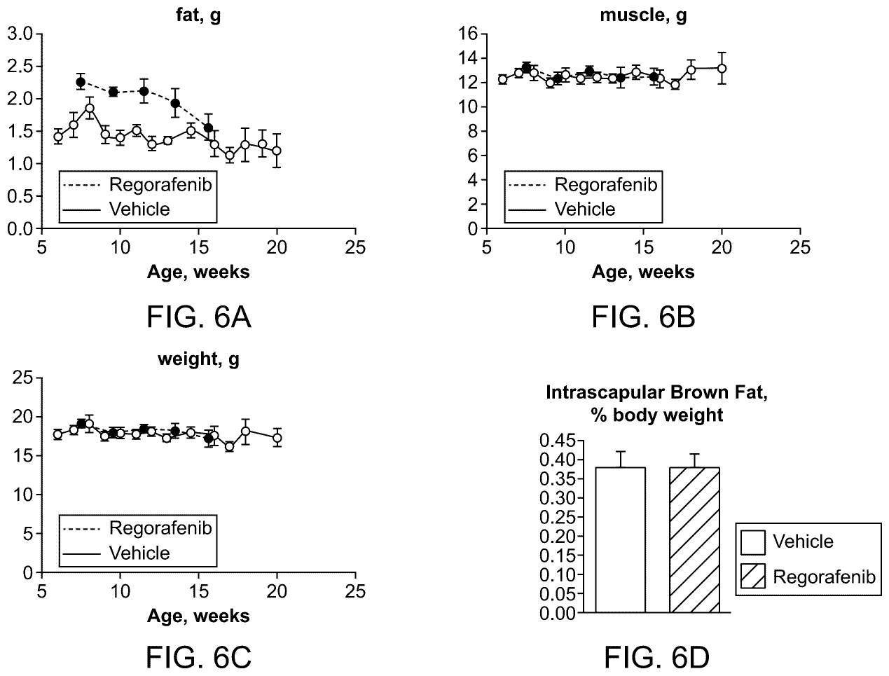

The body fat of the mice was measured by an Echo MM. The mice treated with Regorafenib did not show an increase in body fat, muscle, or weight compared to the control group (FIG. 6A-FIG. 6C). There was also no alteration of intrascapular brown adipose tissue mass (FIG. 6D).

### Example 7. Infigratinib, Which Targets FGF-Receptors Does Not Increase (Brown) Fat, Muscle or Body Mass in Progeria Model

Infigratinib (Truseltiq) is used as FGFR-inhibiting therapy for cholangiocarcinoma. It inhibits FGFR1, FGFR4, and FGFR2 with an IC₅₀ of less than 2 nM.

Zmpste24−/− mice were dosed with either 5 mg/kg infigratinib for 56 days, 5 days on, 2 days off (QD5) or vehicle alone. The dose was administered via oral gavage as a suspension of 18% (w/v) PEG400 2% (w/v) Tween-80 in water. The vehicle alone was 18% (w/v) PEG400 2% (w/v) Tween-80 in water.

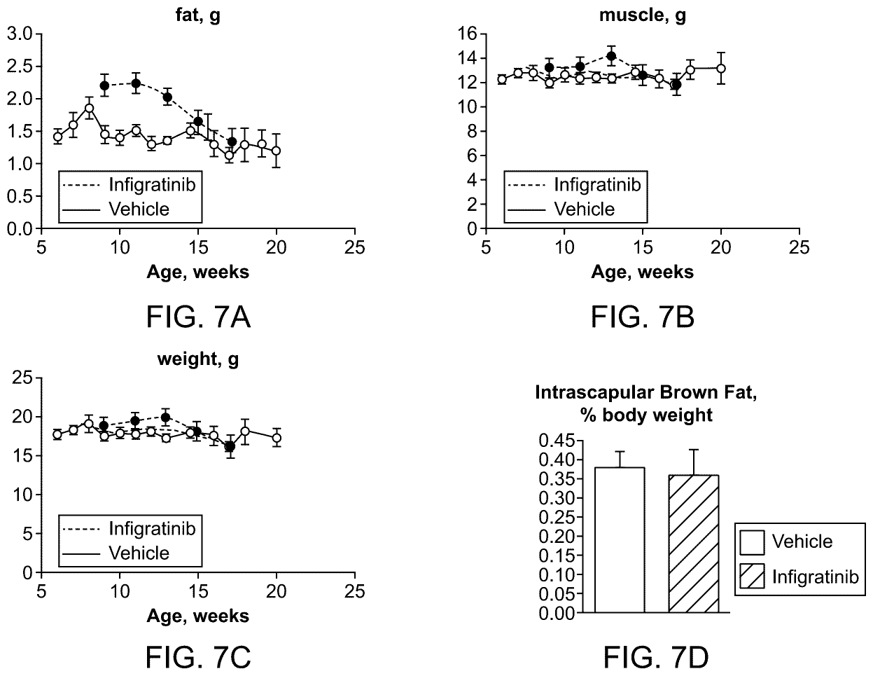

The body fat of the mice was measure by an Echo MRI. The mice treated with infigratinib did not show an increase in body fat, lean muscle, or weight compared to the control group (FIG. 7A-C). There was also no alteration of intrascapular brown adipose tissue mass (FIG. 7D).

### Example 8. A Selective Inhibitor of VEGFR2 and a Selective Inhibitor of FGFR1 Recapitulate the Effects of a Selective Inhibitor of VEGFR2 and FGFR1

Zmpste24−/− mice are dosed with a selective inhibitor of VEGFR2 chosen from Table 3 and a selective inhibitor of FGFR1 chosen from Table 2 for 56 days, 5 days on, 2 days off (QD5), or vehicle alone. The dose is administered via oral gavage as a suspension of 18% (w/v) PEG400 2% Tween-80 (w/v) in water . The vehicle alone is 18% (w/v) PEG400 2% (w/v) Tween-80 in water.

The body fat of the mice is measured by an Echo MRI. The mice that are treated with a selective VEGFR2 inhibitor and a selective FGFR1 inhibitor show an increase in body fat over the course of the treatment.

The lean muscle mass and body weight of the mice are also measured. The mice treated with a selective VEGFR2 inhibitor and a selective FGFR1 inhibitor show an increase in lean muscle mass and body weight compared to the mice that received the vehicle only.

Samples of intrascapular brown adipose tissue from mice treated with a selective VEGFR2 inhibitor and a selective FGFR1 inhibitor are processed for H & E staining. The tissue samples show this treatment increases lipid content in intrascapular brown adipose tissue compared to the mice that receive the vehicle alone.

Intrascapular brown fat mass is also measured and makes up a higher percent of the body weight of the mice treated with a selective VEGFR2 inhibitor and a selective FGFR1 inhibitor compared to mice treated with the vehicle.

The mice that are treated with a selective VEGFR2 inhibitor and a selective FGFR1 inhibitor are also assessed for bone loss in the vertebrae and femur. Bone loss is scored by a pathologist using the criteria in Example 2. The mice treated with a selective VEGFR2 inhibitor and a selective FGFR1 inhibitor have lower bone loss scores compared to mice treated with the vehicle.

### Example 9. A Selective Inhibitor of VEGFR2 and a Selective Inhibitor of FGFR1 Decrease Liver Lipid Content and Increase Storage in Subcutaneous Depot in a Mouse Model of Diet Induced Fatty Liver Disease

26 week old male C57BL6 mice are fed a high fat, high cholesterol, high fructose diet for 23 weeks. The diet provides 40 Kcal % fat from palm oil, 20% kcal % fructose and 2% cholesterol. The mice are then either treated with a selective inhibitor of VEGFR2 and a selective inhibitor of FGFR1 administered in 10% (w/v) DMSO, 4% (w/v) Tween-80, 86% (w/v) PBS, or the vehicle alone, 10% (w/v) DMSO, 4% (w/v) Tween-80, 86% PBS. The mice are dosed for 28 days, once a day, via intraperitoneal injection

At the end of treatment, H&E stained liver samples show mice treated with a selective inhibitor of VEGFR2 and a selective inhibitor of FGFR1 have a decrease in liver lipid content and a decrease in the size of adipocytes in the epididymal fat pad compared to mice that received the vehicle. The mice treatment treated with a selective inhibitor of VEGFR2 and a selective inhibitor of FGFR1 also have an increase in the thickness of the abdominal subcutaneous adipose tissue.

In another experiment, in addition to the two groups of mice above that are fed a high fat, high cholesterol diet, a third group of mice are fed a standard diet and administered the vehicle alone. The group fed the fatty liver diet and treated with a selective inhibitor of VEGFR2 and a selective inhibitor of FGFR1 have less of an increase in body weight, liver mass, serum cholesterol, serum triglycerides, and blood urea nitrogen compared to the mice fed with the fatty liver diet and treated with vehicle compared to the mice fed a standard diet and treated with vehicle.

TABLE 1
List of selective inhibitors of VEGFR2 and FGFR1

| | | | | targets | | |
|---|---|---|---|---|---|---|
| VEGFR2, | FGFR1, | min | avg | IC50 < | Compound | |
| rIC50 | rIC50 | IC50 μM | rIC50 | 10 μM | ID | Compound Name |
| 1.0 | 1.0 | 0.046 | 1.0 | 4 | 11477626 | [4-(2,4-Difluoro-5-methoxycarbamoyl-phenylamino)-5- |
| | | | | | | isopropyl-pyrrolo[2,1-f][1,2,4]triazin-6-yl]-carbamic acid |
| | | | | | | tetrahydro-furan-2-ylmethyl ester |
| 0.9 | 1.1 | 0.025 | 1.0 | 3 | 11234052 | Brivanib alaninate (Brivanib) |
| 1.1 | 1.0 | 0.033 | 1.0 | 7 | 11488852 | 2,4-Difluoro-5-[5-isopropyl-6-(5-trifluoromethyl- |
| | | | | | | [1,3,4]oxadiazol-2-yl)-pyrrolo[2,1-f][1,2,4]triazin-4- |
| | | | | | | ylamino]-N-methoxy-benzamide |
| 1.3 | 0.8 | 0.012 | 1.0 | 3 | 67161103 | 5-[(1-Ethylpiperidin-4-yl)amino]-3-[1H-imidazol-2- |
| | | | | | | yl(phenyl)methylidene]-1H-indol-2-one |
| 1.1 | 1.0 | 0.046 | 1.0 | 7 | 11328182 | 2,4-Difluoro-5-[5-isopropyl-6-(5-methyl-4H-[1,2,4]triazol- |
| | | | | | | 3-yl)-pyrrolo[2,1-f][1,2,4]triazin-4-ylamino]-N-methoxy- |
| | | | | | | benzamide |
| 1.0 | 1.1 | 0.007 | 1.0 | 7 | 9982235 | 5-[6-(5-Dimethylamino-[1,3,4]oxadiazol-2-yl)-5-isopropyl- |
| | | | | | | pyrrolo[2,1-f][1,2,4]triazin-4-ylamino]-2,4-difluoro-N- |
| | | | | | | methoxy-benzamide |
| 1.0 | 1.1 | 0.007 | 1.1 | 7 | 11419379 | 2,4-Difluoro-5-[5-isopropyl-6-(3-methyl-[1,2,4]oxadiazol- |
| | | | | | | 5-yl)-pyrrolo[2,1-f][1,2,4]triazin-4-ylamino]-N-methoxy- |
| | | | | | | benzamide |
| 0.7 | 1.5 | 0.030 | 1.1 | 2 | 5289418 | 3-[(3-(2-Carboxyethyl)-4-methylpyrrol-2-YL)methylene]- |
| | | | | | | 2-indolinone (SU5402) |
| 1.2 | 1.0 | 0.007 | 1.1 | 7 | 10478569 | 2,4-Difluoro-5-[5-isopropyl-6-(5-methyl-[1,3,4]oxadiazol- |
| | | | | | | 2-yl)-pyrrolo[2,1-f][1,2,4]triazin-4-ylamino]-N-methoxy- |
| | | | | | | benzamide |
| 1.0 | 1.2 | 0.017 | 1.1 | 7 | 10073832 | 5-[6-(5-Difluoromethyl-[1,3,4]oxadiazol-2-yl)-5-isopropyl- |
| | | | | | | pyrrolo[2,1-f][1,2,4]triazin-4-ylamino]-2,4-difluoro-N- |
| | | | | | | methoxy-benzamide |
| 0.6 | 1.7 | 0.003 | 1.1 | 3 | 135488014 | 3-[Benzimidazol-2-ylidene-(3-fluorophenyl)methyl]-5-[(1- |
| | | | | | | ethylpiperidin-4-yl)amino]-1H-indol-2-ol |
| 1.3 | 1.0 | 0.339 | 1.2 | 5 | 68209068 | N-(Cyclopropylmethyl)-3-(1-methylpyrazol-4-yl)-N- |
| | | | | | | phenylquinoxalin-6-amine |
| 1.4 | 1.0 | 0.054 | 1.2 | 7 | 11464544 | 2,4-Difluoro-5-(5-isopropyl-6-[1,3,4]oxadiazol-2-yl- |
| | | | | | | pyrrolo[2,1-f][1,2,4]triazin-4-ylamino)-N-methoxy- |
| | | | | | | benzamide |
| 0.6 | 1.8 | 0.083 | 1.2 | 8 | 11396738 | 7-(4-Fluoroanilino)-1-[(1R,3R)-3-hydroxycyclopentyl]-3- |
| | | | | | | (4-methoxyphenyl)-4H-pyrimido[4,5-d]pyrimidin-2-one |
| 1.0 | 1.5 | 0.004 | 1.2 | 7 | 11465545 | 5-[6-(5-Cyclopropyl-[1,3,4]oxadiazol-2-yl)-5-isopropyl- |
| | | | | | | pyrrolo[2,1-f][1,2,4]triazin-4-ylamino]-2,4-difluoro-N- |
| | | | | | | methoxy-benzamide |
| 0.5 | 2.0 | 0.061 | 1.2 | 4 | 44449577 | 6-(5-Methyl-1,3,4-oxadiazol-2-yl)-5-propan-2-yl-N-(1H- |
| | | | | | | pyrrolo[2,3-b]pyridin-5-yl)pyrrolo[2,1-f][1,2,4]triazin-4- |
| | | | | | | amine |
| 1.3 | 1.3 | 3.981 | 1.3 | 5 | 89447546 | N-(Cyclopropylmethyl)-N-[3-methoxy-5- |
| | | | | | | (trifluoromethyl)phenyl]-3-(1-methylpyrazol-4- |
| | | | | | | yl)quinoxalin-6-amine |
| 0.7 | 2.0 | 0.004 | 1.3 | 3 | 135546228 | 3-[Benzimidazol-2-ylidene-(3,5-difluorophenyl)methyl]-5- |
| | | | | | | [(1-ethylpiperidin-4-yl)amino]-1H-indol-2-ol |
| 1.7 | 1.0 | 0.005 | 1.4 | 7 | 5330158 | 1,6-Naphthyridine 76 |
| 1.0 | 1.8 | 0.025 | 1.4 | 7 | 11455031 | 2,4-Difluoro-5-{5-isopropyl-6-[5-(2,2,2-trifluoro-ethyl)- |
| | | | | | | [1,3,4]oxadiazol-2-yl]-pyrrolo[2,1-f][1,2,4]triazin-4- |
| | | | | | | ylamino}-N-methoxy-benzamide |
| 0.4 | 2.4 | 0.028 | 1.4 | 7 | 9910609 | (+)-(1R,3R)-1-(3-Hydroxy-cyclopentyl)-3-(4-methoxy- |
| | | | | | | phenyl)-7-phenylamino-3,4-dihydro-1H-pyrimido[4,5- |
| | | | | | | d]pyrimidin-2-one |
| 1.9 | 1.0 | 0.003 | 1.4 | 5 | 5330157 | 1,6-Naphthyridine 75 |
| 0.5 | 2.5 | 0.033 | 1.5 | 7 | 11407794 | (−)-(1S,3S)-1-(3-Hydroxy-cyclopentyl)-3-(4-methoxy- |
| | | | | | | phenyl)-7-phenylamino-3,4-dihydro-1H-pyrimido[4,5- |
| | | | | | | d]pyrimidin-2-one |
| 1.9 | 1.0 | 0.417 | 1.5 | 4 | 68209477 | N-(Cyclopropylmethyl)-N-[3-[(dimethylamino)methyl]-5- |
| | | | | | | methoxyphenyl]-3-(1-methylpyrazol-4-yl)quinoxalin-6- |
| | | | | | | amine |
| 1.4 | 1.6 | 0.871 | 1.5 | 5 | 134154171 | N-[3-(Aminomethyl)-5-methoxyphenyl]-N- |
| | | | | | | (cyclopropylmethyl)-3-(1-methylpyrazol-4-yl)quinoxalin- |
| | | | | | | 6-amine; hydrochloride |
| 1.1 | 1.9 | 0.150 | 1.5 | 6 | 155563388 | Chembl4573547 |
| 2.0 | 1.0 | 0.003 | 1.5 | 5 | 5330159 | 1,6-Naphthyridine 77 |
| 2.0 | 1.0 | 0.003 | 1.5 | 5 | 5330129 | 1,6-Naphthyridine 19 |
| 2.0 | 1.0 | 0.002 | 1.5 | 5 | 5330136 | 1,6-Naphthyridine 26 |
| 1.1 | 2.0 | 0.603 | 1.5 | 5 | 68209276 | N-(Cyclopropylmethyl)-N-[3-(difluoromethoxy)phenyl]-3- |
| | | | | | | (1-methylpyrazol-4-yl)quinoxalin-6-amine |
| 2.4 | 0.7 | 0.257 | 1.6 | 5 | 134134095 | N′-(3,4-Difluoro-5-methoxyphenyl)-N′-[3-(1- |
| | | | | | | methylpyrazol-4-yl)quinoxalin-6-yl]-N-(2,2,2- |
| | | | | | | trifluoroethyl)propane-1,3-diamine; hydrochloride |
| 1.0 | 2.2 | 0.002 | 1.6 | 7 | 10436729 | 2,4-Difluoro-5-[5-isopropyl-6-(5-methanesulfonylmethyl- |
| | | | | | | [1,3,4]oxadiazol-2-yl)-pyrrolo[2,1-f][1,2,4]triazin-4- |
| | | | | | | ylamino]-N-methoxy-benzamide |
| 0.4 | 2.8 | 0.024 | 1.6 | 4 | 135398512 | 5-(2-Chlorophenyl)-7-fluoro-1,2-dihydro-8-methoxy-3- |
| | | | | | | methylpyrazolo(3,4-b)(1,4)benzodiazepine |
| 1.3 | 2.0 | 0.501 | 1.6 | 5 | 68208764 | [3-[Cyclopropylmethyl-[3-(1-methylpyrazol-4- |
| | | | | | | yl)quinoxalin-6-yl]amino]-5-methoxyphenyl]-pyrrolidin-1- |
| | | | | | | ylmethanone |
| 0.6 | 2.7 | 0.003 | 1.6 | 4 | 67161862 | 5-[(1-Ethylpiperidin-4-yl)amino]-3-[(2-fluorophenyl)-(5- |
| | | | | | | methyl-1H-imidazol-2-yl)methylidene]-1H-indol-2-one |
| 1.0 | 2.3 | 0.004 | 1.7 | 7 | 11155938 | 5-[6-(5-Cyclopropylmethyl-[1,3,4]oxadiazol-2-yl)-5- |
| | | | | | | isopropyl-pyrrolo[2,1-f][1,2,4]triazin-4-ylamino]-2,4- |
| | | | | | | difluoro-N-methoxy-benzamide |
| 1.0 | 2.3 | 0.003 | 1.7 | 4 | 67162590 | 3-[(3,5-Difluorophenyl)-(5-methyl-1H-imidazol-2- |
| | | | | | | yl)methylidene]-5-[(1-ethylpiperidin-4-yl)amino]-1H- |
| | | | | | | indol-2-one |
| 1.2 | 2.2 | 0.006 | 1.7 | 4 | 67163022 | 5-[(1-Ethylpiperidin-4-yl)amino]-3-[(3-fluorophenyl)-(1H- |
| | | | | | | imidazol-2-yl)methylidene]-1H-indol-2-one |
| 3.0 | 0.3 | 0.200 | 1.7 | 4 | 5329165 | Indolin-2-one deriv. 9c |
| 1.1 | 2.2 | 0.032 | 1.7 | 5 | 135476706 | Methyl (3Z)-2-oxo-3-[phenyl-[4-(piperidin-1- |
| | | | | | | ylmethyl)anilino]methylidene]-1H-indole-6-carboxylate |
| 2.0 | 1.5 | 0.012 | 1.7 | 4 | 89447212 | N-(3,5-Dimethoxyphenyl)-N-[3-(3-methylimidazol-4- |
| | | | | | | yl)prop-2-ynyl]-3-(1-methylpyrazol-4-yl)quinoxalin-6- |
| | | | | | | amine |
| 1.7 | 1.8 | 0.006 | 1.8 | 3 | 135430397 | (3z)-5-[(1-Ethylpiperidin-4-Yl)amino]-3-[(5-Methoxy-1h- |
| | | | | | | Benzimidazol-2-YL)(Phenyl)methylidene]-1,3-Dihydro-2h- |
| | | | | | | Indol-2-One |
| 2.4 | 1.2 | 0.110 | 1.8 | 7 | 155520650 | Chembl4449189 |
| 0.5 | 3.1 | 0.110 | 1.8 | 6 | 134146580 | [5-Amino-1-(1H-indol-5-yl)pyrazol-4-yl]-(1H-indol-2- |
| | | | | | | yl)methanone |
| 0.5 | 3.1 | 0.025 | 1.8 | 3 | 22273644 | 5-Isopropyl-6-(5-methyl-1,3,4-oxadiazol-2-yl)-N-(2- |
| | | | | | | methyl-1H-pyrrolo[2,3-b]pyridin-5-yl)pyrrolo[2,1- |
| | | | | | | f][1,2,4]triazin-4-amine |
| 1.6 | 2.0 | 0.005 | 1.8 | 3 | 135507297 | 3-[Benzimidazol-2-ylidene-(4-methylphenyl)methyl]-5- |
| | | | | | | [(1-ethylpiperidin-4-yl)amino]-1H-indol-2-ol |
| 1.0 | 2.6 | 0.002 | 1.8 | 7 | 11374515 | 5-[6-(5-Ethyl-[1,3,4]oxadiazol-2-yl)-5-isopropyl- |
| | | | | | | pyrrolo[2,1-f][1,2,4]triazin-4-ylamino]-2,4-difluoro-N- |
| | | | | | | methoxy-benzamide |
| 2.0 | 1.7 | 0.003 | 1.8 | 3 | 135405015 | 3-[Benzimidazol-2-ylidene(phenyl)methyl]-5-[(1- |
| | | | | | | ethylpiperidin-4-yl)amino]-1H-indol-2-ol |
| 2.7 | 1.0 | 0.003 | 1.8 | 5 | 5330135 | 1,6-Naphthyridine 25 |
| 1.6 | 2.0 | 0.016 | 1.8 | 4 | 135458467 | 2-(6-Fluoro-1H-indazol-3-yl)-5-(4-(piperidin-1- |
| | | | | | | yl)piperidin-1-yl)-1H-benzo[d]imidazole |
| 1.9 | 2.0 | 0.007 | 1.9 | 3 | 135488027 | 3-[Benzimidazol-2-ylidene-(4-methoxyphenyl)methyl]-5- |
| | | | | | | [(1-ethylpiperidin-4-yl)amino]-1H-indol-2-ol |
| 2.3 | 1.7 | 0.479 | 2.0 | 4 | 68209467 | N-(Cyclopropylmethyl)-N-[3-methoxy-5-[2- |
| | | | | | | (methylamino)ethoxy]phenyl]-3-(1-methylpyrazol-4- |
| | | | | | | yl)quinoxalin-6-amine |
| 3.2 | 0.8 | 0.006 | 2.0 | 5 | 68209431 | 3-Methoxy-N-methyl-5-[[3-(1-methylpyrazol-4- |
| | | | | | | yl)quinoxalin-6-yl]-[2-(propan-2- |
| | | | | | | ylamino)ethyl]amino]benzamide |
| 2.0 | 2.0 | 0.002 | 2.0 | 4 | 67163677 | XL999; XL-999; XL 999 |
| 1.4 | 2.7 | 0.275 | 2.1 | 4 | 68208796 | 3-N-(Cyclopropylmethyl)-1-N-methyl-3-N-[3-(1- |
| | | | | | | methylpyrazol-4-yl)quinoxalin-6-yl]benzene-1,3-diamine |
| 1.0 | 3.1 | 0.002 | 2.1 | 7 | 11352215 | 2,4-Difluoro-5-[6-(5-isobutyl-[1,3,4]oxadiazol-2-yl)-5- |
| | | | | | | isopropyl-pyrrolo[2,1-f][1,2,4]triazin-4-ylamino]-N- |
| | | | | | | methoxy-benzamide |
| 0.7 | 3.5 | 0.007 | 2.1 | 6 | 141475848 | Methyl 2-hydroxy-3-[N-[4-[2-methyl-5-[(4- |
| | | | | | | methylpiperazin-1-yl)methyl]pyrrol-1-yl]phenyl]-C- |
| | | | | | | phenylcarbonimidoyl]-1H-indole-6-carboxylate |
| 2.8 | 1.4 | 0.037 | 2.1 | 5 | 68209353 | N-(3,5-Dimethoxyphenyl)-3-(1-methylpyrazol-4-yl)-N-[(2- |
| | | | | | | pyridin-4-ylcyclopropyl)methyl]quinoxalin-6-amine |
| 4.1 | 0.3 | 0.044 | 2.2 | 5 | 5330125 | 3-(3,5-Dimethoxyphenyl)-7-N-[3-(4-methylpiperazin-1- |
| | | | | | | yl)propyl]-1,6-naphthyridine-2,7-diamine |
| 2.0 | 2.4 | 0.005 | 2.2 | 6 | 56945277 | 9-Chloro-2-[3-[3-(dimethylamino)propyl]anilino]-5,7- |
| | | | | | | dihydropyrimido[5,4-d][1]benzazepine-6-thione |
| 3.5 | 1.0 | 0.006 | 2.3 | 5 | 5330167 | Pyrido[2,3-d]pyrimidine 103 |
| 3.6 | 1.0 | 0.020 | 2.3 | 5 | 5330169 | Pyrido[2,3-d]pyrimidine 105 |
| 1.0 | 3.6 | 0.001 | 2.3 | 7 | 10388612 | 2,4-Difluoro-5-[5-isopropyl-6-(5-methyl-oxazol-2-yl)- |
| | | | | | | pyrrolo[2,1-f][1,2,4]triazin-4-ylamino]-N-methoxy- |
| | | | | | | benzamide |
| 2.4 | 2.2 | 0.004 | 2.3 | 4 | 134131810 | N-(3,5-Dimethoxyphenyl)-N-[3-(2,5-dimethylimidazol-1- |
| | | | | | | yl)propyl]-3-(1-methylpyrazol-4-yl)quinoxalin-6- |
| | | | | | | amine; hydrochloride |
| 4.0 | 0.7 | 0.250 | 2.3 | 4 | 86293280 | N-[3-[2-[6-(2-Chlorophenyl)-2-[4- |
| | | | | | | (diethylamino)butylamino]-7-oxopyrido[2,3-d]pyrimidin- |
| | | | | | | 8-yl]ethyl]phenyl]prop-2-enamide |
| 3.6 | 1.1 | 0.003 | 2.4 | 5 | 134136522 | N-(3,5-Dimethoxyphenyl)-3-(1-methylpyrazol-4-yl)-N-(2- |
| | | | | | | piperazin-1-ylethyl)quinoxalin-6-amine; hydrochloride |
| 2.3 | 2.5 | 0.010 | 2.4 | 5 | 135456078 | Methyl (3Z)-3-[[4-[2-(dimethylamino)ethyl- |
| | | | | | | methylsulfonylamino]anilino]-phenylmethylidene]-2-oxo- |
| | | | | | | 1H-indole-6-carboxylate |
| 3.9 | 0.9 | 0.008 | 2.4 | 5 | 89447553 | 4-[3-(3,5-Dimethoxy-N-[3-(1-methylpyrazol-4- |
| | | | | | | yl)quinoxalin-6-yl]anilino)prop-1-ynyl]pyridine-3- |
| | | | | | | carbonitrile |
| 3.8 | 1.2 | 0.003 | 2.5 | 5 | 134135434 | N-[(5-Chlorothiophen-2-yl)methyl]-N-(3,5- |
| | | | | | | dimethoxyphenyl)-3-[1-(2-piperazin-1-ylethyl)pyrazol-4- |
| | | | | | | yllquinoxalin-6-amine; hydrochloride |
| 3.3 | 1.7 | 0.251 | 2.5 | 5 | 89447459 | N′-(3,5-Dichlorophenyl)-N′-[3-(1-methylpyrazol-4- |
| | | | | | | yl)quinoxalin-6-yl]-N-(2,2,2-trifluoroethyl)propane-1,3- |
| | | | | | | diamine |
| 4.6 | 0.5 | 0.050 | 2.5 | 5 | 68209034 | 2-[4-[7-[N-(Cyclopropylmethyl)anilino]quinoxalin-2- |
| | | | | | | yl]pyrazol-1-yllethanol |
| 4.1 | 1.0 | 0.015 | 2.6 | 5 | 5330166 | Pyrido[2,3-d]pyrimidine 102 |
| 4.8 | 0.4 | 0.046 | 2.6 | 5 | 5330131 | 7-N-[4-(Diethylamino)butyl]-3-(3,5-dimethoxyphenyl)- |
| | | | | | | 1,6-naphthyridine-2,7-diamine |
| 3.8 | 1.4 | 0.020 | 2.6 | 5 | 89447470 | N-(3,5-Dimethoxyphenyl)-N-[3-(4-methoxypyrimidin-2- |
| | | | | | | yl)propyl]-3-(1-methylpyrazol-4-yl)quinoxalin-6-amine |
| 2.3 | 2.9 | 0.028 | 2.6 | 5 | 135522918 | Methyl 3-[[4-[(dimethylamino)methyl]anilino]- |
| | | | | | | phenylmethylidene]-2-oxo-1H-indole-6-carboxylate |
| 4.8 | 0.7 | 0.002 | 2.8 | 5 | 68209105 | 3-Fluoro-N-methyl-5-[[3-(1-methylpyrazol-4- |
| | | | | | | yl)quinoxalin-6-yl]-[2-(propan-2- |
| | | | | | | ylamino)ethyllamino]benzamide |
| 4.2 | 1.3 | 0.178 | 2.8 | 5 | 68208875 | N-(Cyclopropylmethyl)-N-[3-(1,3-dioxolan-2-yl)-5- |
| | | | | | | methoxyphenyl]-3-(1-methylpyrazol-4-yl)quinoxalin-6- |
| | | | | | | amine |
| 1.9 | 3.7 | 0.003 | 2.8 | 4 | 89447352 | N-[[(2S)-1-[3-(3,5-Dimethoxy-N-[3-(1-methylpyrazol-4- |
| | | | | | | yl)quinoxalin-6-yl]anilino)propyl]pyrrolidin-2-yl]methyl]- |
| | | | | | | 1,1,1-trifluoromethanesulfonamide |
| 4.7 | 1.1 | 0.007 | 2.9 | 5 | 68208860 | 3-Methoxy-N-methyl-5-[2-(propan-2-ylamino)ethyl-[3-(1- |
| | | | | | | propan-2-ylpyrazol-4-yl)quinoxalin-6-yl]amino]benzamide |
| 1.0 | 4.8 | 0.027 | 2.9 | 7 | 11432911 | 5-{6-[5-(Difluoro-methanesulfonyl-methyl)- |
| | | | | | | [1,3,4]oxadiazol-2-yl]-5-isopropyl-pyrrolo[2,1- |
| | | | | | | f][1,2,4]triazin-4-ylamino}-2,4-difluoro-N-methoxy- |
| | | | | | | benzamide |
| 5.2 | 0.7 | 0.004 | 3.0 | 5 | 68209078 | N-[2-(3-Aminopiperidin-1-yl)ethyl]-N-(3,5- |
| | | | | | | dimethoxyphenyl)-3-(1-methylpyrazol-4-yl)quinoxalin-6- |
| | | | | | | amine |
| 4.5 | 1.5 | 0.025 | 3.0 | 5 | 89447351 | N-(3,5-Dimethoxyphenyl)-N-[(E)-3-(4-methoxypyrimidin- |
| | | | | | | 2-yl)prop-2-enyl]-3-(1-methylpyrazol-4-yl)quinoxalin-6- |
| | | | | | | amine |
| 2.4 | 3.6 | 0.005 | 3.0 | 5 | 135528963 | Methyl (3Z)-3-[[4-[acetyl-[2- |
| | | | | | | (dimethylamino)ethyl]amino]anilino]-phenylmethylidene]- |
| | | | | | | 2-oxo-1H-indole-6-carboxylate |
| 4.3 | 1.7 | 0.006 | 3.0 | 9 | 156010414 | Methyl N-[4-[7-amino-3-(3,4-dimethoxyphenyl)-5-(1- |
| | | | | | | methylpiperidin-4-yl)oxypyrazolo[1,5-a]pyrimidin-6- |
| | | | | | | yl]phenyl]carbamate |
| 1.0 | 5.0 | 0.020 | 3.0 | 7 | 5329033 | Anilinoquinazoline deriv. 35 |
| 5.0 | 1.0 | 0.001 | 3.0 | 5 | 5330127 | 1,6-Naphthyridine 17 |
| 5.1 | 0.9 | 0.019 | 3.0 | 5 | 89446990 | Tert-butyl (1S,4S)-5-[2-(3,5-dimethoxy-N-[3-(1- |
| | | | | | | methylpyrazol-4-yl)quinoxalin-6-yl]anilino)ethyl]-2,5- |
| | | | | | | diazabicyclo[2.2.1]heptane-2-carboxylate |
| 3.6 | 2.5 | 0.010 | 3.0 | 4 | 25031915 | Lucitanib |
| 3.6 | 2.5 | 0.010 | 3.0 | 4 | 57379018 | 6-((7-((1-Aminocyclopropyl)methoxy)-6-methoxyquinolin- |
| | | | | | | 4-yl)oxy)-N-methyl-1-naphthamide hydrochloride |
| 5.8 | 1.0 | 0.033 | 3.4 | 5 | 5330168 | Pyrido[2,3-d]pyrimidine 104 |
| 1.6 | 5.3 | 0.013 | 3.5 | 9 | 135476717 | Nintedanib esylate |
| 5.9 | 1.1 | 0.035 | 3.5 | 5 | 68208866 | [(2S)-1-(3,5-Dimethoxy-N-[3-(1-methylpyrazol-4- |
| | | | | | | yl)quinoxalin-6-yl]anilino)-3-methoxypropan-2-yl] acetate |
| 6.2 | 0.9 | 0.003 | 3.5 | 4 | 68209101 | 3-[2-Aminoethyl-[3-(1-methylpyrazol-4-yl)quinoxalin-6- |
| | | | | | | yl]amino]-5-methoxy-N-methylbenzamide |
| 5.9 | 1.2 | 0.002 | 3.5 | 5 | 89447202 | N-(3,5-Dimethoxyphenyl)-3-(1-methylpyrazol-4-yl)-N-[3- |
| | | | | | | [4-(trifluoromethyl)piperidin-1-yl]propyl]quinoxalin-6- |
| | | | | | | amine |
| 0.4 | 6.7 | 0.001 | 3.6 | 6 | 9811611 | 3-((4-Bromo-2,6-difluorobenzyl)oxy)-5-(3-(4-(pyrrolidin- |
| | | | | | | 1-yl)butyl)ureido)isothiazole-4-carboxamide |
| 5.4 | 1.7 | 0.004 | 3.6 | 5 | 89447488 | N-(3,5-Dimethoxyphenyl)-N-[3-(2-methylimidazol-1- |
| | | | | | | yl)propyl]-3-(1-methylpyrazol-4-yl)quinoxalin-6-amine |
| 1.4 | 5.7 | 0.070 | 3.6 | 4 | 44299203 | 3-[3-(Morpholinomethyl)-4,5,6, 7-tetrahydro-1H-indole-2- |
| | | | | | | ylmethylene]-5-(ethylsulfonyl)-2,3-dihydro-1H-indole-2- |
| | | | | | | one |
| 5.4 | 1.9 | 0.020 | 3.6 | 5 | 68208941 | 2-(3-Methoxy-N-[3-(1-methylpyrazol-4-yl)quinoxalin-6- |
| | | | | | | yl]anilino)ethanol |
| 5.8 | 1.6 | 0.360 | 3.7 | 5 | 24949852 | Epihematoxylol |
| 6.2 | 1.3 | 0.062 | 3.8 | 5 | 68208938 | N-[3-(5-Aminopyrazin-2-yl)prop-2-ynyl]-N-(3,5- |
| | | | | | | dimethoxyphenyl)-3-(1-methylpyrazol-4-yl)quinoxalin-6- |
| | | | | | | amine |
| 6.5 | 1.1 | 0.010 | 3.8 | 5 | 89447236 | 2-[3-[2-Aminoethyl-[7-(1-methylpyrazol-4-yl)quinoxalin- |
| | | | | | | 2-yl]amino]-5-fluorophenoxy ethanol |
| 6.0 | 1.6 | 0.006 | 3.8 | 5 | 89447552 | N-[3-(3-Aminopyridin-2-yl)prop-2-ynyl]-N-(3,5- |
| | | | | | | dimethoxyphenyl)-3-(1-methylpyrazol-4-yl)quinoxalin-6- |
| | | | | | | amine |
| 6.6 | 1.0 | 0.025 | 3.8 | 5 | 68208967 | N-(3,5-Dimethoxyphenyl)-N-[3-(3-methoxypyridin-2- |
| | | | | | | yl)propyl]-3-(1-methylpyrazol-4-yl)quinoxalin-6-amine |
| 6.5 | 1.5 | 0.012 | 4.0 | 5 | 68209193 | N-(3,5-Dimethoxyphenyl)-3-(1-methylpyrazol-4-yl)-N- |
| | | | | | | [(Z)-3-pyrimidin-2-ylprop-2-enyl]quinoxalin-6-amine |
| 7.2 | 0.9 | 0.006 | 4.1 | 5 | 134138981 | N′-(3,5-Dimethoxyphenyl)-N-methyl-N′-[3-[1-(oxan-4- |
| | | | | | | ylmethyl)pyrazol-4-yl]quinoxalin-6-yl]ethane-1,2- |
| | | | | | | diamine; hydrochloride |
| 5.6 | 2.8 | 0.000 | 4.2 | 5 | 134156584 | [5-Amino-1-(2-chloro-5-hydroxyphenyl)pyrazol-4-yl]-[5- |
| | | | | | | (morpholin-4-ylmethyl)-1H-indol-2-yl]methanone |
| 7.5 | 1.0 | 0.002 | 4.3 | 5 | 5330128 | 1,6-Naphthyridine 18 |
| 7.5 | 1.0 | 0.000 | 4.3 | 5 | 5330130 | 1-Tert-butyl-3-[3-(3,5-dimethoxyphenyl)-7-[4-(4- |
| | | | | | | methylpiperazin-1-yl)butylamino]-1,6-naphthyridin-2- |
| | | | | | | ylurea |
| 7.9 | 0.6 | 0.002 | 4.3 | 5 | 134157989 | N′-(2-Chloro-3,5-dimethoxyphenyl)-N′-[3-(1- |
| | | | | | | methylpyrazol-4-yl)quinoxalin-6-yl]-N-propan-2-ylethane- |
| | | | | | | 1,2-diamine; hydrochloride |
| 7.6 | 1.0 | 0.004 | 4.3 | 5 | 68209364 | N-(3,5-Dimethoxyphenyl)-3-(1-methylpyrazol-4-yl)-N-(3- |
| | | | | | | piperazin-1-ylpropyl)quinoxalin-6-amine |
| 3.0 | 5.7 | 0.003 | 4.3 | 5 | 135405065 | Methyl (3Z)-3-[[4-[acetyl-[3- |
| | | | | | | (dimethylamino)propyl]amino]anilino]- |
| | | | | | | phenylmethylidenel-2-oxo-1H-indole-6-carboxylate |
| 7.1 | 1.7 | 0.003 | 4.4 | 4 | 134132327 | 3-[4-[3-[4-[7-[N-(Cyclopropylmethyl)-3,5- |
| | | | | | | dimethoxyanilino]quinoxalin-2-yl]pyrazol-1- |
| | | | | | | yllpropyl]piperazin-1-yl]propan-1-ol; hydrochloride |
| 8.3 | 0.4 | 0.001 | 4.4 | 5 | 136211077 | 5-[(R)-1-(3,5-Dichloropyridine-4-yl)ethoxy]-3-[5-(4- |
| | | | | | | methylpiperazine-1-yl)-1H-benzoimidazole-2-yl]-1H- |
| | | | | | | indazole |
| 5.8 | 3.0 | 0.010 | 4.4 | 5 | 135410282 | 4-Amino-3-(1H-benzo[d]imidazol-2-yl)quinolin-2(1H)-one |
| 8.3 | 0.5 | 0.002 | 4.4 | 5 | 134147714 | 3-[4-[3-[4-[7-[3,5-Dimethoxy-N-[2-(propan-2- |
| | | | | | | ylamino)ethyl]anilino]quinoxalin-2-yl]pyrazol-1- |
| | | | | | | yllpropyl]piperazin-1-yl]propan-1-ol; hydrochloride |
| 8.5 | 0.9 | 0.001 | 4.7 | 5 | 88888594 | 2-(2,6-Difluoro-3,5-dimethoxy-N-[3-(1-methylpyrazol-4- |
| | | | | | | yl)quinoxalin-6-yl]anilino)ethanol |
| 9.1 | 0.5 | 0.002 | 4.8 | 4 | 68209292 | [4-[7-[3,5-Dimethoxy-N-[2-(propan-2- |
| | | | | | | ylamino)ethyl]anilino]quinoxalin-2-yl]-2-methylpyrazol-3- |
| | | | | | | yl]methanol |
| 9.0 | 1.0 | 0.001 | 5.0 | 5 | 5329004 | 1,6-Naphthyridine deriv. 20 |
| 9.5 | 0.6 | 0.002 | 5.1 | 5 | 134136463 | N-(2-Chloro-3,5-dimethoxyphenyl)-3-(1-methylpyrazol-4- |
| | | | | | | yl)-N-(3-pyrrolidin-1-ylpropyl)quinoxalin-6- |
| | | | | | | amine; hydrochloride |
| 2.2 | 8.0 | 0.011 | 5.1 | 4 | 135452890 | Methyl 3-[N-[4-[2-(dimethylamino)ethyl- |
| | | | | | | methylcarbamoyl]phenyl]-C-phenylcarbonimidoyl]-2- |
| | | | | | | hydroxy-1H-indole-6-carboxylate |
| 9.1 | 1.1 | 0.004 | 5.1 | 4 | 134156950 | N-[2-(1,4-Diazepan-1-yl)ethyl]-N-(3,5-dimethoxyphenyl)- |
| | | | | | | 3-(1-methylpyrazol-4-yl)quinoxalin-6-amine; oxalic acid |
| 9.8 | 0.5 | 0.002 | 5.1 | 5 | 87410340 | N′-(2,6-Difluoro-3,5-dimethoxyphenyl)-N′-[3-(1- |
| | | | | | | methylpyrazol-4-yl)quinoxalin-6-yl]-N-propan-2-ylethane- |
| | | | | | | 1,2-diamine |
| 7.8 | 2.5 | 0.011 | 5.1 | 5 | 68209120 | N-(3,5-Dimethoxyphenyl)-N-[(3-methyloxetan-3- |
| | | | | | | yl)methyl]-3-(1-methylpyrazol-4-yl)quinoxalin-6-amine |
| 1.3 | 9.0 | 0.006 | 5.2 | 4 | 135456052 | Methyl (3Z)-3-[[4-[acetyl-[2-(dimethylamino)-2- |
| | | | | | | oxoethyl]amino]anilino]-phenylmethylidene]-2-oxo-1H- |
| | | | | | | indole-6-carboxylate |
| 1.7 | 8.7 | 0.037 | 5.2 | 6 | 138393067 | Chembl4213341 |
| 0.5 | 10.0 | 0.300 | 5.3 | 7 | 5329034 | Anilinoquinazoline deriv. 36 |
| 10.3 | 0.3 | 0.033 | 5.3 | 4 | 68005732 | [5-Amino-1-(1H-benzimidazol-6-yl)-1H-pyrazol-4-yl]-1H- |
| | | | | | | indol-2-ylMethanone |
| 7.2 | 3.4 | 0.850 | 5.3 | 5 | 44578561 | Hematoxylone |
| 8.7 | 2.0 | 0.002 | 5.4 | 5 | 68209457 | N-Cyclopropyl-N′-(3,5-dimethoxyphenyl)-N′-[3-(1- |
| | | | | | | methylpyrazol-4-yl)quinoxalin-6-yl]ethane-1,2-diamine |
| 9.3 | 1.4 | 0.013 | 5.4 | 5 | 68208997 | N-(3,5-Dimethoxyphenyl)-3-(1-methylpyrazol-4-yl)-N-(3- |
| | | | | | | pyrimidin-2-ylpropyl)quinoxalin-6-amine |
| 10.0 | 0.8 | 0.013 | 5.4 | 5 | 68209322 | N′-(3,5-Dimethoxyphenyl)-N′-[3-[5- |
| | | | | | | [(dimethylamino)methyl]-1-methylpyrazol-4- |
| | | | | | | yl]quinoxalin-6-yl]-N-propan-2-ylethane-1,2-diamine |
| 10.2 | 0.7 | 0.001 | 5.4 | 4 | 88888623 | N′-(2,6-Difluoro-3,5-dimethoxyphenyl)-N′-[3-(1- |
| | | | | | | methylpyrazol-4-yl)quinoxalin-6-yl]-N-(2,2,2- |
| | | | | | | trifluoroethyl)propane-1,3-diamine |
| 0.4 | 10.6 | 0.018 | 5.5 | 3 | 134155961 | [5-Amino-1-(2-methyl-1H-indol-5-yl)pyrazol-4-yl]-(1H- |
| | | | | | | indol-2-yl)methanone |
| 9.5 | 1.5 | 0.220 | 5.5 | 6 | 442514 | Haematoxylin |
| 10.2 | 0.8 | 0.010 | 5.5 | 4 | 89447494 | N′-(3,5-Dimethoxyphenyl)-N′-[3-(5-ethyl-1-methylpyrazol- |
| | | | | | | 4-yl)quinoxalin-6-yl]-N-(2,2,2-trifluoroethyl)ethane-1,2- |
| | | | | | | diamine |
| 6.1 | 5.0 | 0.010 | 5.6 | 5 | 135837837 | Methyl 3-[N-[4- |
| | | | | | | [[dimethylcarbamoyl(methyl)amino]methyl]phenyl]-C- |
| | | | | | | phenylcarbonimidoyl]-2-hydroxy-1H-indole-6-carboxylate |
| 9.3 | 1.8 | 0.004 | 5.6 | 4 | 68208730 | 3-Methoxy-N-methyl-5-[[3-(1-methylpyrazol-4- |
| | | | | | | yl)quinoxalin-6-yl]-(3-pyrrolidin-1- |
| | | | | | | ylpropyl)amino]benzamide |
| 9.8 | 1.4 | 0.003 | 5.6 | 5 | 68208940 | N-(3,5-Dimethoxyphenyl)-3-(1-methylpyrazol-4-yl)-N-(3- |
| | | | | | | pyridin-4-ylpropyl)quinoxalin-6-amine |
| 9.3 | 1.9 | 0.003 | 5.6 | 5 | 68208791 | 1-[3-(3,5-Dimethoxy-N-[3-(1-propan-2-ylpyrazol-4- |
| | | | | | | yl)quinoxalin-6-yl]anilino)propyl]pyrrolidin-2-one |
| 8.4 | 3.0 | 0.001 | 5.7 | 10 | 11167602 | Regorafenib |

TABLE 2
List of selective inhibitors of FGFR1

| | | | Numlow | | |
|---|---|---|---|---|---|
| FGFR1 | Min | Relative | IC50 | Pubchem_ | |
| IC50_μM | IC50_μM | IC50 | Targets | CID | Compound Name |
| 0.0029 | 0.065 | 0.044615385 | 1 | 66555232 | [5-amino-1-(2-ethyl-3H-benzimidazol-5-yl)pyrazol-4-yl]-(1H- |
| | | | | | indol-2-yl)methanone |
| 0.0003 | 0.0026 | 0.115384615 | 2 | 46944259 | LY-2874455 |
| 0.0003631 | 0.002884 | 0.125901526 | 3 | 68208989 | N′-(3,5-dimethoxyphenyl)-N′-[3-(1-piperidin-4-ylpyrazol-4- |
| | | | | | yl)quinoxalin-6-yl]-N-propan-2-ylethane-1,2-diamine |
| 0.0007762 | 0.005754 | 0.134897463 | 3 | 134139707 | N′-[3-[1-(3-aminopropyl)pyrazol-4-yl]quinoxalin-6-yl]-N′-(3,5- |
| | | | | | dimethoxyphenyl)-N-propan-2-ylethane-1,2-diamine;2,2,2- |
| | | | | | trifluoroacetic acid |
| 0.001585 | 0.00912 | 0.17379386 | 4 | 88888615 | N′-(2,6-dichloro-3,5-dimethoxyphenyl)-N′-[3-(1-methylpyrazol- |
| | | | | | 4-yl)quinoxalin-6-yl]-N-propan-2-ylethane-1,2-diamine |
| 0.0006761 | 0.003162 | 0.213820367 | 4 | 68208933 | tert-butyl 4-[4-[7-[3,5-dimethoxy-N-[2-(propan-2- |
| | | | | | ylamino)ethyl]anilino]quinoxalin-2-yl]pyrazol-1-yl]piperidine-1- |
| | | | | | carboxylate |
| 0.0007586 | 0.00309 | 0.245501618 | 4 | 68208882 | N-(3,5-dimethoxyphenyl)-N-[(5,5-dimethylmorpholin-3- |
| | | | | | yl)methyl]-3-(1-methylpyrazol-4-yl)quinoxalin-6-amine |
| 0.0004571 | 0.001738 | 0.263003452 | 3 | 89447047 | N-[3,5-bis(trideuteriomethoxy)phenyl]-1,1,2,2-tetradeuterio-N- |
| | | | | | [3-(1-methylpyrazol-4-yl)quinoxalin-6-yl]-N′-propan-2-ylethane- |
| | | | | | 1,2-diamine |
| 0.033 | 0.12 | 0.275 | 4 | 68005732 | [5-amino-1-(1H-benzimidazol-6-yl)-1H-pyrazol-4-yl]-1H-indol- |
| | | | | | 2-ylMethanone |
| 0.001413 | 0.004898 | 0.288485096 | 4 | 68208877 | 2-N-(3,5-dimethoxyphenyl)-2-N-[3-(1-methylpyrazol-4- |
| | | | | | yl)quinoxalin-6-yl]-1-N-propan-2-ylpropane-1,2-diamine |
| 0.044 | 0.15 | 0.293333333 | 5 | 5330125 | 3-(3,5-dimethoxyphenyl)-7-N-[3-(4-methylpiperazin-1- |
| | | | | | yl)propyl]-1,6-naphthyridine-2,7-diamine |
| 0.001349 | 0.004571 | 0.295121418 | 4 | 68209490 | 2-N-(3,5-dimethoxyphenyl)-2-N-[3-(1-methylpyrazol-4- |
| | | | | | yl)quinoxalin-6-yl]propane-1,2-diamine |
| 0.0029 | 0.0094 | 0.308510638 | 2 | 68005621 | [5-amino-1-(2-methyl-3H-benzimidazol-5-yl)pyrazol-4-yl]-[6- |
| | | | | | [(4,4-difluoropiperidin-1-yl)methyl]-1H-indol-2-yl]methanone |
| 0.007079 | 0.02188 | 0.323537477 | 3 | 68209111 | N-(3,5-dimethoxyphenyl)-N-[(1-ethylimidazol-2-yl)methyl]-3- |
| | | | | | (1-methylpyrazol-4-yl)quinoxalin-6-amine |
| 0.003388 | 0.01047 | 0.323591213 | 3 | 68209458 | 1-N-(3,5-dimethoxyphenyl)-1-N-[3-(1-methylpyrazol-4- |
| | | | | | yl)quinoxalin-6-yl]propane-1,2-diamine |
| 0.0004467 | 0.001349 | 0.331134173 | 4 | 68208888 | 1-[3-(3,5-dimethoxy-N-[3-(1-methylpyrazol-4-yl)quinoxalin-6- |
| | | | | | yl]anilino)propyl]azetidin-3-ol |
| 0.000912 | 0.002754 | 0.331154684 | 4 | 118014711 | N′-[3-methoxy-5-(trideuteriomethoxy)phenyl]-N′-[3-(1- |
| | | | | | methylpyrazol-4-yl)quinoxalin-6-yl]-N-propan-2-ylethane-1,2- |
| | | | | | diamine |
| 0.2 | 0.6 | 0.333333333 | 4 | 5329165 | Indolin-2-one deriv. 9c |
| 0.001585 | 0.004677 | 0.338892452 | 4 | 68209154 | N′-(3,5-dimethoxyphenyl)-N′-[3-(1,5-dimethylpyrazol-4- |
| | | | | | yl)quinoxalin-6-yl]-N-propan-2-ylethane-1,2-diamine |
| 0.0005888 | 0.001698 | 0.346760895 | 4 | 134135471 | N-(3,5-dimethoxyphenyl)-3-(1-methylpyrazol-4-yl)-N-[3-[(2S)- |
| | | | | | 2-methylpyrrolidin-1-yl]propyl]quinoxalin-6- |
| | | | | | amine;hydrochloride |
| 0.046 | 0.13 | 0.353846154 | 5 | 5330131 | 7-N-[4-(diethylamino)butyl]-3-(3,5-dimethoxyphenyl)-1,6- |
| | | | | | naphthyridine-2,7-diamine |
| 0.00263 | 0.007413 | 0.354782139 | 4 | 67467703 | N-(3,5-dimethoxyphenyl)-N-[3-(4-methoxypyrimidin-2-yl)prop- |
| | | | | | 2-ynyl]-3-(1-methylpyrazol-4-yl)quinoxalin-6-amine |
| 0.002512 | 0.006918 | 0.363110726 | 3 | 134147847 | N′-(3,5-dimethoxyphenyl)-N′-[3-[1-methyl-3- |
| | | | | | (trifluoromethyl)pyrazol-4-yl]quinoxalin-6-yl]-N-propan-2- |
| | | | | | ylethane-1,2-diamine;hydrochloride |
| 0.001413 | 0.00389 | 0.363239075 | 3 | 68209043 | N′-(3,5-dimethoxyphenyl)-N-methyl-N′-[3-(1-methylpyrazol-4- |
| | | | | | yl)quinoxalin-6-yl]-N-propan-2-ylethane-1,2-diamine |
| 0.0003 | 0.0008 | 0.375 | 2 | 138617501 | 1-[(3S)-3-[4-amino-5-(7-methoxy-5-methyl-1-benzothiophen-2- |
| | | | | | yl)pyrrolo[2,3-d]pyrimidin-7-yl]pyrrolidin-1-yl]prop-2-en-1-one |
| 0.001905 | 0.005012 | 0.380087789 | 4 | 68209466 | N-[3-(2-aminopyrimidin-4-yl)prop-2-ynyl]-N-(3,5- |
| | | | | | dimethoxyphenyl)-3-(1-methylpyrazol-4-yl)quinoxalin-6-amine |
| 0.000871 | 0.002291 | 0.380183326 | 4 | 118014709 | N′-(3,5-dimethoxyphenyl)-N-propan-2-yl-N′-[3-[1- |
| | | | | | (trideuteriomethyl)pyrazol-4-yl]quinoxalin-6-yl]ethane-1,2- |
| | | | | | diamine |
| 0.001514 | 0.003802 | 0.398211468 | 3 | 134145521 | 1-N-(3,5-dimethoxyphenyl)-1-N-[3-(1-methylpyrazol-4- |
| | | | | | yl)quinoxalin-6-yl]-2-N-propan-2-ylpropane-1,2- |
| | | | | | diamine;hydrochloride |
| 0.002089 | 0.005129 | 0.40729187 | 4 | 68208876 | (2R)-2-N-(3,5-dimethoxyphenyl)-2-N-[3-(1-methylpyrazol-4- |
| | | | | | yl)quinoxalin-6-yl]propane-1,2-diamine |
| 0.0014 | 0.0034 | 0.411764706 | 4 | 68004741 | [5-amino-1-(2-methyl-1H-benzimidazol-5-yl)-1H-pyrazol-4-yl]- |
| | | | | | (6-morpholin-4-ylmethyl-1H-indol-2-yl)-methanone |
| 0.00123 | 0.002818 | 0.436479773 | 4 | 134157816 | N′-[3-methoxy-5-(trideuteriomethoxy)phenyl]-N-propan-2-yl-N′- |
| | | | | | [3-(1H-pyrazol-4-yl)quinoxalin-6-yl]ethane-1,2- |
| | | | | | diamine;hydrochloride |
| 0.00182 | 0.004169 | 0.436555529 | 3 | 134153451 | N-(3,5-dimethoxyphenyl)-N-[3-(4-methoxypiperidin-1- |
| | | | | | yl)propyl]-3-(1-methylpyrazol-4-yl)quinoxalin-6-amine |
| 0.001445 | 0.003236 | 0.446538937 | 4 | 68208658 | N′-(3,5-dimethoxyphenyl)-N-methyl-N′-[3-(1-methylpyrazol-4- |
| | | | | | yl)quinoxalin-6-yl]ethane-1,2-diamine |
| 0.01995 | 0.04467 | 0.446608462 | 3 | 68209183 | 1-[4-[3-Aminopropyl-[3-(1-methylpyrazol-4-yl)quinoxalin-6- |
| | | | | | yl]amino]-2,6-dimethoxyphenyl]-2,2,2-trifluoroethanol |
| 0.002884 | 0.006457 | 0.44664705 | 4 | 134147170 | N′-(3,5-dimethoxy-2-methylphenyl)-N′-[3-(1-methylpyrazol-4- |
| | | | | | yl)quinoxalin-6-yl]-N-propan-2-ylethane-1,2- |
| | | | | | diamine;hydrochloride |
| 0.00263 | 0.005888 | 0.446671196 | 3 | 89447340 | N-(2,6-dichloro-3,5-dimethoxyphenyl)-N-[3-(3-methoxypyridin- |
| | | | | | 2-yl)prop-2-ynyl]-3-(1-methylpyrazol-4-yl)quinoxalin-6-amine |
| 0.0009 | 0.002 | 0.45 | 5 | 136211077 | 5-[(R)-1-(3,5-Dichloropyridine-4-yl)ethoxy]-3-[5-(4- |
| | | | | | methylpiperazine-1-yl)-1H-benzoimidazole-2-yl]-1H-indazole |
| 0.0007586 | 0.00166 | 0.456987952 | 4 | 134143004 | N-(3,5-dimethoxyphenyl)-3-(1-methylpyrazol-4-yl)-N-(3- |
| | | | | | pyrrolidin-1-ylpropyl)quinoxalin-6-amine;hydrochloride |
| 0.001047 | 0.002291 | 0.457005674 | 4 | 89447290 | 2-[4-[7-[3,5-dimethoxy-N-(3-pyrrolidin-1- |
| | | | | | ylpropyl)anilino]quinoxalin-2-yl]pyrazol-1-yl]-N- |
| | | | | | methylacetamide |
| 0.0006 | 0.0013 | 0.461538462 | 3 | 118295624 | 8-(3-(4-acryloylpiperazin-1-yl)propyl)-6-(2,6-dichloro-3,5- |
| | | | | | dimethoxyphenyl)-2-(methylamino)pyrido[2,3-d]pyrimidin- |
| | | | | | 7(8H)-one |
| 0.07586 | 0.16218 | 0.467751881 | 4 | 89447430 | 4-[3-(3,5-dimethoxy-N-[3-(1-methylpyrazol-4-yl)quinoxalin-6- |
| | | | | | yl]anilino)prop-1-ynyl]pyridine-2-carbonitrile |
| 0.001349 | 0.002884 | 0.467753121 | 4 | 68209505 | N′-(7-methoxy-1,3-benzodioxol-5-yl)-N′-[3-(1-methylpyrazol-4- |
| | | | | | yl)quinoxalin-6-yl]-N-propan-2-ylethane-1,2-diamine |
| 0.0006166 | 0.001318 | 0.467830046 | 4 | 89447189 | N-(3,5-dimethoxyphenyl)-N-[(5-methyl-1,2-oxazol-3-yl)methyl]- |
| | | | | | 3-(1-methylpyrazol-4-yl)quinoxalin-6-amine |
| 0.001148 | 0.002399 | 0.478532722 | 4 | 134135843 | N-(3,5-dimethoxyphenyl)-N-[3-(2-methylpiperidin-1-yl)propyl]- |
| | | | | | 3-(1-methylpyrazol-4-yl)quinoxalin-6-amine;hydrochloride |
| 0.05012 | 0.10471 | 0.478655334 | 5 | 68209034 | 2-[4-[7-[N-(cyclopropylmethyl)anilino]quinoxalin-2-yl]pyrazol- |
| | | | | | 1-yl]ethanol |
| 0.0005495 | 0.001148 | 0.478658537 | 4 | 134135131 | N-(3,5-dimethoxyphenyl)-3-(1-methylpyrazol-4-yl)-N-(3- |
| | | | | | piperidin-1-ylpropyl)quinoxalin-6-amine;hydrochloride |
| 0.001549 | 0.003236 | 0.478677379 | 3 | 68209147 | N′-(3,5-dimethoxyphenyl)-N,N-dimethyl-N′-[3-(1- |
| | | | | | methylpyrazol-4-yl)quinoxalin-6-yl]ethane-1,2-diamine |
| 0.002692 | 0.005623 | 0.478747999 | 4 | 134145360 | N-[2-[(1S,4S)-2,5-diazabicyclo[2.2.1]heptan-2-yl]ethyl]-N-(3,5- |
| | | | | | dimethoxyphenyl)-3-(1-methylpyrazol-4-yl)quinoxalin-6- |
| | | | | | amine;hydrochloride |
| 0.001622 | 0.003388 | 0.478748524 | 4 | 134148952 | N′-(3,5-dimethoxyphenyl)-N-propan-2-yl-N′-[3-(1H-pyrazol-4- |
| | | | | | yl)quinoxalin-6-yl]ethane-1,2-diamine;hydrochloride |
| 0.001413 | 0.002951 | 0.478820739 | 3 | 134142615 | N′-(3,5-dimethoxyphenyl)-N′-[3-[1-(1-methylpiperidin-4- |
| | | | | | yl)pyrazol-4-yl]quinoxalin-6-yl]-N-propan-2-ylethane-1,2- |
| | | | | | diamine;hydrochloride |
| 0.00123 | 0.002512 | 0.489649682 | 4 | 134146514 | N-(3,5-dimethoxyphenyl)-N-[2-(2-methylpiperidin-1-yl)ethyl]-3- |
| | | | | | (1-methylpyrazol-4-yl)quinoxalin-6-amine;hydrochloride |
| 0.00182 | 0.003715 | 0.489905787 | 3 | 68208841 | 2-[(3,5-dimethoxy-N-[3-(1-methylpyrazol-4-yl)quinoxalin-6- |
| | | | | | yl]anilino)methyl]-N,N-dimethylimidazole-1-sulfonamide |
| 0.0002 | 0.0004 | 0.5 | 3 | 51039095 | Azd-4547 |
| 0.001778 | 0.003548 | 0.501127396 | 5 | 87410340 | N′-(2,6-difluoro-3,5-dimethoxyphenyl)-N′-[3-(1-methylpyrazol- |
| | | | | | 4-yl)quinoxalin-6-yl]-N-propan-2-ylethane-1,2-diamine |
| 0.002399 | 0.004786 | 0.501253656 | 4 | 68209030 | N-(3,5-dimethoxyphenyl)-N-[2-(2,6-dimethylpiperidin-1- |
| | | | | | yl)ethyl]-3-(1-methylpyrazol-4-yl)quinoxalin-6-amine |
| 0.00955 | 0.01905 | 0.501312336 | 4 | 89447337 | 2-[[5-[(3,5-dimethoxy-N-[3-(1-methylpyrazol-4-yl)quinoxalin-6- |
| | | | | | yl]anilino)methyl]-1,3,4-oxadiazol-2-yl]amino]ethanol |
| 0.006457 | 0.01288 | 0.501319876 | 4 | 89447497 | N-[3-(2-amino-6-chloropyrimidin-4-yl)prop-2-ynyl]-N-(3,5- |
| | | | | | dimethoxyphenyl)-3-(1-methylpyrazol-4-yl)quinoxalin-6-amine |
| 0.0007244 | 0.001413 | 0.512668082 | 4 | 68209028 | N-[3-(3-aminopyrrolidin-1-yl)propyl]-N-(3,5-dimethoxyphenyl)- |
| | | | | | 3-(1-methylpyrazol-4-yl)quinoxalin-6-amine |
| 0.003802 | 0.007413 | 0.512882774 | 4 | 89447141 | (2S)-1-N-(3,5-dimethoxyphenyl)-1-N-[3-(1-methylpyrazol-4- |
| | | | | | yl)quinoxalin-6-yl]propane-1,2-diamine |
| 0.00195 | 0.003802 | 0.512887954 | 4 | 88888566 | N′-(3,5-dimethoxyphenyl)-N-(2-fluoroethyl)-N′-[3-(1- |
| | | | | | methylpyrazol-4-yl)quinoxalin-6-yl]ethane-1,2-diamine |
| 0.00138 | 0.00263 | 0.524714829 | 3 | 89447157 | 2-[4-[7-[N-(cyclopropylmethyl)-3,5- |
| | | | | | dimethoxyanilino]quinoxalin-2-yl]pyrazol-1-yl]-N-(2-pyrrolidin- |
| | | | | | 1-ylethyl)acetamide |
| 0.0006166 | 0.001175 | 0.524765957 | 3 | 68208813 | N′-(3,5-dimethoxyphenyl)-N′-[3-(1H-pyrazol-4-yl)quinoxalin-6- |
| | | | | | yl]propane-1,3-diamine |
| 0.001778 | 0.003388 | 0.524793388 | 3 | 134139651 | N′-(3,5-dimethoxyphenyl)-N′-[3-(5-ethyl-1-methylpyrazol-4- |
| | | | | | yl)quinoxalin-6-yl]-N-propan-2-ylethane-1,2- |
| | | | | | diamine;hydrochloride |
| 0.03236 | 0.06166 | 0.524813493 | 3 | 68208812 | N-[(2-chlorophenyl)methyl]-N-(3,5-dimethoxyphenyl)-3-(1- |
| | | | | | methylpyrazol-4-yl)quinoxalin-6-amine |
| 0.002188 | 0.004169 | 0.524826097 | 5 | 134147714 | 3-[4-[3-[4-[7-[3,5-dimethoxy-N-[2-(propan-2- |
| | | | | | ylamino)ethyl]anilino]quinoxalin-2-yl]pyrazol-1- |
| | | | | | yl]propyl]piperazin-1-yl]propan-1-ol;hydrochloride |
| 0.001096 | 0.002042 | 0.536728697 | 4 | 68208743 | N′-(3,5-dimethoxyphenyl)-N′-[3-(1-methylpyrazol-4- |
| | | | | | yl)quinoxalin-6-yl]-N-propan-2-ylpropane-1,3-diamine |
| 0.001622 | 0.00302 | 0.537086093 | 4 | 68208850 | N′-(3,5-dimethoxyphenyl)-N,N-diethyl-N′-[3-(1-methylpyrazol- |
| | | | | | 4-yl)quinoxalin-6-yl]ethane-1,2-diamine |
| 0.005495 | 0.01 | 0.5495 | 3 | 68208937 | N-(3,5-dimethoxyphenyl)-3-(1-methylpyrazol-4-yl)-N-[[3- |
| | | | | | [(propan-2-ylamino)methyl]oxetan-3-yl]methyl]quinoxalin-6- |
| | | | | | amine |
| 0.002188 | 0.003981 | 0.549610651 | 4 | 68208878 | N-(3,5-dimethoxyphenyl)-3-(1-methylpyrazol-4-yl)-N- |
| | | | | | (morpholin-3-ylmethyl)quinoxalin-6-amine |
| 0.00182 | 0.003311 | 0.549682875 | 4 | 68209292 | [4-[7-[3,5-dimethoxy-N-[2-(propan-2- |
| | | | | | ylamino)ethyl]anilino]quinoxalin-2-yl]-2-methylpyrazol-3- |
| | | | | | yl]methanol |
| 0.0007762 | 0.00138 | 0.562463768 | 4 | 134146349 | N′-(3,5-dimethoxyphenyl)-N′-[3-(1-ethylpyrazol-4-yl)quinoxalin- |
| | | | | | 6-yl]-N-propan-2-ylethane-1,2-diamine;hydrochloride |
| 0.002692 | 0.004786 | 0.562473882 | 4 | 89447371 | N-(3,5-dimethoxyphenyl)-3-(1-methylpyrazol-4-yl)-N-(3- |
| | | | | | pyridin-4-ylprop-2-ynyl)quinoxalin-6-amine |
| 0.00166 | 0.002951 | 0.562521179 | 4 | 89447499 | N-[3-(4-aminopyridin-2-yl)prop-2-ynyl]-N-(3,5- |
| | | | | | dimethoxyphenyl)-3-(1-methylpyrazol-4-yl)quinoxalin-6-amine |
| 0.001072 | 0.001905 | 0.562729659 | 4 | 134144550 | 2-[4-[7-[3,5-dimethoxy-N-(3-pyrrolidin-1- |
| | | | | | ylpropyl)anilino]quinoxalin-2-yl]pyrazol-1- |
| | | | | | yl]ethanol;hydrochloride |
| 0.002754 | 0.004786 | 0.575428333 | 3 | 134156865 | N′-(2-chloro-5-methoxyphenyl)-N-propan-2-yl-N′-[3-(1H- |
| | | | | | pyrazol-4-yl)quinoxalin-6-yl]ethane-1,2-diamine;hydrochloride |
| 0.00182 | 0.003162 | 0.575585073 | 5 | 134136463 | N-(2-chloro-3,5-dimethoxyphenyl)-3-(1-methylpyrazol-4-yl)-N- |
| | | | | | (3-pyrrolidin-1-ylpropyl)quinoxalin-6-amine;hydrochloride |
| 0.00182 | 0.003162 | 0.575585073 | 4 | 68208975 | N-(3,5-dimethoxyphenyl)-N-[2-[3-(dimethylamino)pyrrolidin-1- |
| | | | | | yl]ethyl]-3-(1-methylpyrazol-4-yl)quinoxalin-6-amine |
| 0.0008318 | 0.001413 | 0.588676575 | 3 | 89447336 | N-(3,5-dimethoxyphenyl)-N-[(2-methyl-1,3-thiazol-4- |
| | | | | | yl)methyl]-3-[1-(2-piperazin-1-ylethyl)pyrazol-4-yl]quinoxalin- |
| | | | | | 6-amine |
| 0.001175 | 0.001995 | 0.588972431 | 4 | 68209116 | N′-(3,5-dimethoxyphenyl)-N-(1-fluoro-2-methylpropan-2-yl)-N′- |
| | | | | | [3-(1-methylpyrazol-4-yl)quinoxalin-6-yl]propane-1,3-diamine |
| 0.00138 | 0.002291 | 0.602357049 | 3 | 68209409 | 3-[1-(3-aminopropyl)pyrazol-4-yl]-N-(cyclopropylmethyl)-N- |
| | | | | | (3,5-dimethoxyphenyl)quinoxalin-6-amine |
| 0.02818 | 0.04677 | 0.602522985 | 4 | 68208920 | [6-[3-(3,5-dimethoxy-N-[3-(1-methylpyrazol-4-yl)quinoxalin-6- |
| | | | | | yl]anilino)prop-1-ynyl]pyridin-2-yl]methanol |
| 0.002188 | 0.003631 | 0.602588819 | 4 | 68209451 | N′-(3,5-dimethoxyphenyl)-N-ethyl-N′-[3-(1-methylpyrazol-4- |
| | | | | | yl)quinoxalin-6-yl]ethane-1,2-diamine |
| 0.001259 | 0.002089 | 0.602680708 | 4 | 68208744 | N-(3,5-dimethoxyphenyl)-3-(1-methylpyrazol-4-yl)-N-[2-(1- |
| | | | | | propan-2-ylpiperidin-4-yl)ethyl]quinoxalin-6-amine |
| 0.0006166 | 0.001023 | 0.602737048 | 4 | 68209344 | N′-(3,5-dimethoxyphenyl)-N′-[3-(1-propan-2-ylpyrazol-4- |
| | | | | | yl)quinoxalin-6-yl]propane-1,3-diamine |
| 0.001072 | 0.001778 | 0.602924634 | 4 | 134156523 | N-(3,5-dimethoxyphenyl)-3-(1-methylpyrazol-4-yl)-N-(2- |
| | | | | | pyrrolidin-1-ylethyl)quinoxalin-6-amine;hydrochloride |
| 0.004677 | 0.007586 | 0.616530451 | 4 | 68209268 | (2R)-1-N-(3,5-dimethoxyphenyl)-1-N-[3-(1-methylpyrazol-4- |
| | | | | | yl)quinoxalin-6-yl]propane-1,2-diamine |
| 0.002089 | 0.003388 | 0.616587957 | 3 | 134150563 | N-(3-chloro-5-methoxyphenyl)-3-(1-methylpyrazol-4-yl)-N-(3- |
| | | | | | pyrrolidin-1-ylpropyl)quinoxalin-6-amine;hydrochloride |
| 0.002188 | 0.003548 | 0.616685457 | 4 | 134145340 | N-(3,5-dimethoxy-2-methylphenyl)-3-(1-methylpyrazol-4-yl)-N- |
| | | | | | (3-pyrrolidin-1-ylpropyl)quinoxalin-6-amine;hydrochloride |
| 0.00195 | 0.003162 | 0.616698292 | 4 | 68208803 | N-(3,5-dimethoxyphenyl)-3-(1-methylpyrazol-4-yl)-N-[(2S)- |
| | | | | | pyrrolidin-2-yl]methyl]quinoxalin-6-amine |
| 0.001413 | 0.002291 | 0.61676124 | 4 | 67467552 | N′-(3,5-dimethoxyphenyl)-N′-[3-(1-methylpyrazol-4- |
| | | | | | yl)quinoxalin-6-yl]propane-1,3-diamine |
| 0.000955 | 0.001514 | 0.630779392 | 4 | 68208768 | N′-(3,5-dimethoxyphenyl)-N-methyl-N′-[3-(1-methylpyrazol-4- |
| | | | | | yl)quinoxalin-6-yl]propane-1,3-diamine |
| 0.002884 | 0.004571 | 0.63093415 | 4 | 134155786 | N-(3,5-dimethoxyphenyl)-N-[3-[3-[(2S,6R)-2,6- |
| | | | | | dimethylmorpholin-4-yl]pyrrolidin-1-yl]propyl]-3-(1- |
| | | | | | methylpyrazol-4-yl)quinoxalin-6-amine;hydrochloride |
| 0.00195 | 0.00309 | 0.631067961 | 4 | 68209460 | [1-[2-(3,5-dimethoxy-N-[3-(1-methylpyrazol-4-yl)quinoxalin-6- |
| | | | | | yl]anilino)ethyl]piperidin-4-yl]methanol |
| 0.001413 | 0.002239 | 0.631085306 | 3 | 134140450 | N-(3,5-dimethoxyphenyl)-N-[3-[3-(methylamino)pyrrolidin-1- |
| | | | | | yl]propyl]-3-(1-methylpyrazol-4-yl)quinoxalin-6- |
| | | | | | amine;hydrochloride |
| 0.002188 | 0.003467 | 0.631093164 | 4 | 89447562 | (2R)-3-(3,5-dimethoxy-N-[3-(1-propan-2-ylpyrazol-4- |
| | | | | | yl)quinoxalin-6-yl]anilino)-1,1,1-trifluoropropan-2-ol |
| 0.001622 | 0.00257 | 0.631128405 | 4 | 68208748 | 2-[4-[7-[3,5-dimethoxy-N-[2- |
| | | | | | (methylamino)ethyl]anilino]quinoxalin-2-yl]pyrazol-1-yl]-N- |
| | | | | | methylacetamide |
| 0.001778 | 0.002754 | 0.645606391 | 4 | 68209461 | N-cyclopentyl-N′-(3,5-dimethoxyphenyl)-N′-[3-(1- |
| | | | | | methylpyrazol-4-yl)quinoxalin-6-yl]ethane-1,2-diamine |
| 0.003236 | 0.005012 | 0.645650439 | 4 | 134135785 | N′-[2-(3,5-dimethoxy-N-[3-(1-methylpyrazol-4-yl)quinoxalin-6- |
| | | | | | yl]anilino)ethyl]-N′-propan-2-ylpropane-1,3- |
| | | | | | diamine;hydrochloride |
| 0.004169 | 0.006457 | 0.645655877 | 4 | 68209117 | N′-(3,5-dimethoxyphenyl)-N′-[3-(1-methylpyrazol-4- |
| | | | | | yl)quinoxalin-6-yl]-N-(3,3,3-trifluoropropyl)ethane-1,2-diamine |
| 0.001549 | 0.002399 | 0.645685702 | 3 | 134155819 | 2-[4-[3-[4-[7-[3,5-dimethoxy-N-[2-(propan-2- |
| | | | | | ylamino)ethyl]anilino]quinoxalin-2-yl]pyrazol-1- |
| | | | | | yl]propyl]piperazin-1-yl]ethanol;hydrochloride |
| 0.002239 | 0.003467 | 0.645803288 | 5 | 134157989 | N′-(2-chloro-3,5-dimethoxyphenyl)-N′-[3-(1-methylpyrazol-4- |
| | | | | | yl)quinoxalin-6-yl]-N-propan-2-ylethane-1,2- |
| | | | | | diamine;hydrochloride |
| 0.0029 | 0.0044 | 0.659090909 | 4 | 68005465 | [5-amino-1-(2-methyl-1H-benzimidazol-5-yl)-1H-pyrazol-4-yl]- |
| | | | | | (5-morpholin-4-ylmethyl-1H-indol-2-yl)-methanone |
| 0.003388 | 0.005129 | 0.660557614 | 4 | 89447050 | (2S)-2-N-(3,5-dimethoxyphenyl)-2-N-[3-(1-methylpyrazol-4- |
| | | | | | yl)quinoxalin-6-yl]propane-1,2-diamine |
| 0.00138 | 0.002089 | 0.660603159 | 4 | 88888623 | N′-(2,6-difluoro-3,5-dimethoxyphenyl)-N′-[3-(1-methylpyrazol- |
| | | | | | 4-yl)quinoxalin-6-yl]-N-(2,2,2-trifluoroethyl)propane-1,3- |
| | | | | | diamine |
| 0.00389 | 0.005888 | 0.660665761 | 5 | 68209078 | N-[2-(3-aminopiperidin-1-yl)ethyl]-N-(3,5-dimethoxyphenyl)-3- |
| | | | | | (1-methylpyrazol-4-yl)quinoxalin-6-amine |
| 0.00389 | 0.005888 | 0.660665761 | 4 | 89447175 | N-[(4-aminophenyl)methyl]-N′-(3,5-dimethoxyphenyl)-N′-[3-(1- |
| | | | | | methylpyrazol-4-yl)quinoxalin-6-yl]-N-propan-2-ylethane-1,2- |
| | | | | | diamine |
| 0.0389 | 0.05888 | 0.660665761 | 3 | 68209333 | [(2S)-3-(3,5-dimethoxy-N-[3-(1-methylpyrazol-4-yl)quinoxalin- |
| | | | | | 6-yl]anilino)-1,1,1-trifluoropropan-2-yl] acetate |
| 0.04786 | 0.07244 | 0.660684705 | 4 | 68208978 | N-(3,5-dimethoxyphenyl)-N-[(Z)-3-(3-methoxypyridin-2- |
| | | | | | yl)prop-2-enyl]-3-(1-methylpyrazol-4-yl)quinoxalin-6-amine |
| 0.00182 | 0.002754 | 0.660856935 | 4 | 134147743 | N-(3,5-dimethoxyphenyl)-3-(1-propan-2-ylpyrazol-4-yl)-N-(3- |
| | | | | | pyrrolidin-1-ylpropyl)quinoxalin-6-amine;hydrochloride |
| 0.001413 | 0.002138 | 0.660898036 | 4 | 68208846 | N-(3,5-dimethoxyphenyl)-3-(1-methylpyrazol-4-yl)-N-(2- |
| | | | | | piperidin-4-ylethyl)quinoxalin-6-amine |
| 0.001413 | 0.002138 | 0.660898036 | 4 | 134146811 | N-(3,5-dimethoxyphenyl)-3-(1-methylpyrazol-4-yl)-N-[3-[(2R)- |
| | | | | | 2-methylpyrrolidin-1-yl]propyl]quinoxalin-6- |
| | | | | | amine;hydrochloride |
| 0.01698 | 0.02512 | 0.675955414 | 4 | 89447417 | 6-[3-(3,5-dimethoxy-N-[3-(1-methylpyrazol-4-yl)quinoxalin-6- |
| | | | | | yl]anilino)prop-1-ynyl]pyridine-2-carbonitrile |
| 0.001479 | 0.002188 | 0.675959781 | 4 | 68208807 | 2-[4-[7-[3,5-dimethoxy-N-[3-(propan-2- |
| | | | | | ylamino)propyl]anilino]quinoxalin-2-yl]pyrazol-1-yl]-N- |
| | | | | | methylacetamide |
| 0.007762 | 0.01148 | 0.676132404 | 4 | 134147581 | 3-methoxy-5-[[3-(1H-pyrazol-4-yl)quinoxalin-6-yl]-[3-(2,2,2- |
| | | | | | trifluoroethylamino)propyl]amino]phenol;hydrochloride |
| 0.001259 | 0.001862 | 0.676154672 | 4 | 134140310 | N-(3-fluoro-5-methoxyphenyl)-3-(1H-pyrazol-4-yl)-N-(3- |
| | | | | | pyrrolidin-1-ylpropyl)quinoxalin-6-amine;hydrochloride |
| 0.00166 | 0.002455 | 0.676171079 | 4 | 134153401 | N-(3,5-dimethoxyphenyl)-3-(1H-pyrazol-4-yl)-N-(2-pyrrolidin- |
| | | | | | 1-ylethyl)quinoxalin-6-amine;hydrochloride |
| 0.25 | 0.369 | 0.677506775 | 4 | 86293280 | N-(3-(2-(6-(2-chlorophenyl)-2-(4-(diethylamino)butylamino)-7- |
| | | | | | oxopyrido[2,3-d]-pyrimidin-8(7H)-yl)ethyl)phenyl)acrylamide |
| 0.89 | 1.3 | 0.684615385 | 5 | 139434105 | 1-(4-Phenoxyphenyl)-3-phenyl-1,3,5-triazinane-2,4,6-trione |
| 0.001023 | 0.001479 | 0.69168357 | 4 | 68209146 | 2-[4-[7-[N-(3-aminopropyl)-3,5-dimethoxyanilino]quinoxalin-2- |
| | | | | | yl]pyrazol-1-yl]-N-methylacetamide |
| 0.01288 | 0.01862 | 0.691729323 | 4 | 89447482 | N-(3,5-dimethoxyphenyl)-N-[3-[3-(methylamino)pyridin-2- |
| | | | | | yl]prop-2-ynyl]-3-(1-methylpyrazol-4-yl)quinoxalin-6-amine |
| 0.001122 | 0.001622 | 0.691738594 | 4 | 68209434 | N′-(3,5-dimethoxyphenyl)-N′-[3-(1-ethylpyrazol-4-yl)quinoxalin- |
| | | | | | 6-yl]ethane-1,2-diamine |
| 0.00309 | 0.004467 | 0.691739422 | 3 | 68208702 | ethyl 2-(3,5-dimethoxy-N-[3-(1-methylpyrazol-4-yl)quinoxalin- |
| | | | | | 6-yl]anilino)acetate |
| 0.001995 | 0.002884 | 0.691747573 | 4 | 89447185 | 2-[4-[3-[N-(2-aminoethyl)-3,5-dimethoxyanilino]quinoxalin-6- |
| | | | | | yl]pyrazol-1-yl]-N-methylacetamide |
| 0.001995 | 0.002884 | 0.691747573 | 4 | 68208907 | 2-[1-[2-(3,5-dimethoxy-N-[3-(1-methylpyrazol-4-yl)quinoxalin- |
| | | | | | 6-yl]anilino)ethyl]piperidin-4-yl]ethanol |
| 0.005623 | 0.008128 | 0.691806102 | 3 | 68208969 | N-(3,5-dimethoxyphenyl)-3-(1-methylpyrazol-4-yl)-N- |
| | | | | | (pyrimidin-2-ylmethyl)quinoxalin-6-amine |
| 0.001549 | 0.002239 | 0.691826708 | 4 | 68209029 | N-(3,5-dimethoxyphenyl)-3-(1-methylpyrazol-4-yl)-N-(2- |
| | | | | | piperidin-1-ylethyl)quinoxalin-6-amine |
| 0.001585 | 0.002291 | 0.691837625 | 4 | 134138403 | N-(3,5-dimethoxyphenyl)-3-(1-methylpyrazol-4-yl)-N-(2- |
| | | | | | pyrazol-1-ylethyl)quinoxalin-6-amine;hydrochloride |
| 0.002344 | 0.003388 | 0.691853601 | 4 | 134132789 | 2-[4-[7-[3,5-dimethoxy-N-[2-(propan-2- |
| | | | | | ylamino)ethyl]anilino]quinoxalin-2-yl]pyrazol-1-yl]-N- |
| | | | | | methylacetamide;hydrochloride |
| 0.005129 | 0.007413 | 0.691892621 | 4 | 68209287 | N-(3,5-dimethoxyphenyl)-3-(1-methylpyrazol-4-yl)-N-(oxolan-2- |
| | | | | | ylmethyl)quinoxalin-6-amine |
| 0.002455 | 0.003548 | 0.691939121 | 4 | 68208927 | N′-(3,5-dimethoxyphenyl)-N′-[3-(1-ethylpyrazol-4-yl)quinoxalin- |
| | | | | | 6-yl]-N-methylethane-1,2-diamine |
| 0.001514 | 0.002188 | 0.691956124 | 4 | 134141677 | N′-(3,5-dimethoxyphenyl)-N′-[3-[1-(oxan-4-ylmethyl)pyrazol-4- |
| | | | | | yl]quinoxalin-6-yl]-N-propan-2-ylethane-1,2- |
| | | | | | diamine;hydrochloride |
| 0.001096 | 0.001549 | 0.70755326 | 4 | 68209090 | N′-(3,5-dimethoxyphenyl)-N′-[3-[1-(2- |
| | | | | | methylsulfonylethyl)pyrazol-4-yl]quinoxalin-6-yl]ethane-1,2- |
| | | | | | diamine |
| 0.006457 | 0.00912 | 0.708004386 | 4 | 89447490 | 2-[3-(3,5-dimethoxy-N-[3-(1-methylpyrazol-4-yl)quinoxalin-6- |
| | | | | | yl]anilino)prop-1-ynyl]pyridine-4-carbonitrile |
| 0.002042 | 0.002884 | 0.708044383 | 4 | 134133926 | N′-(3,5-dimethoxyphenyl)-N-methyl-N′-[3-(1-propan-2- |
| | | | | | ylpyrazol-4-yl)quinoxalin-6-yl]ethane-1,2-diamine;hydrochloride |
| 0.002399 | 0.003388 | 0.708087367 | 4 | 89447343 | [2-[3-(3,5-dimethoxy-N-[3-(1-methylpyrazol-4-yl)quinoxalin-6- |
| | | | | | yl]anilino)prop-1-ynyl]pyridin-4-yl]methanol |
| 0.001413 | 0.001995 | 0.708270677 | 4 | 134157853 | N-[2-(3-aminopyrrolidin-1-yl)ethyl]-N-(3,5-dimethoxyphenyl)-3- |
| | | | | | (1-methylpyrazol-4-yl)quinoxalin-6-amine;hydrochloride |
| 0.001778 | 0.002455 | 0.724236253 | 4 | 89447328 | 2-[3-(3,5-dimethoxy-N-[3-(1-methylpyrazol-4-yl)quinoxalin-6- |
| | | | | | yl]anilino)prop-1-ynyl]pyridine-4-carboxamide |
| 0.001738 | 0.002399 | 0.724468529 | 4 | 134131862 | 2-[4-[7-[3,5-dimethoxy-N-[2- |
| | | | | | (methylamino)ethyl]anilino]quinoxalin-2-yl]pyrazol-1-yl]-N- |
| | | | | | methylacetamide;hydrochloride |
| 0.002291 | 0.003162 | 0.724541429 | 5 | 68209105 | 3-fluoro-N-methyl-5-[[3-(1-methylpyrazol-4-yl)quinoxalin-6-yl]- |
| | | | | | [2-(propan-2-ylamino)ethyl]amino]benzamide |
| 0.002042 | 0.002818 | 0.724627395 | 4 | 88888568 | N-(3,5-dimethoxyphenyl)-3-(1-methylpyrazol-4-yl)-N-prop-2- |
| | | | | | ynylquinoxalin-6-amine |
| 0.01 | 0.0138 | 0.724637681 | 4 | 134132077 | N-[(2-aminophenyl)methyl]-N′-(3,5-dimethoxycyclohexa-2,4- |
| | | | | | dien-1-yl)-N′-[3-(1-methylpyrazol-4-yl)quinoxalin-6-yl]-N- |
| | | | | | propan-2-ylethane-1,2-diamine |
| 0.003802 | 0.005129 | 0.741275102 | 4 | 68209342 | N-(3,5-dimethoxyphenyl)-N-(1,3-dioxolan-2-ylmethyl)-3-(1- |
| | | | | | methylpyrazol-4-yl)quinoxalin-6-amine |
| 0.25704 | 0.34674 | 0.741304724 | 5 | 134134095 | N′-(3,4-difluoro-5-methoxyphenyl)-N′-[3-(1-methylpyrazol-4- |
| | | | | | yl)quinoxalin-6-yl]-N-(2,2,2-trifluoroethyl)propane-1,3- |
| | | | | | diamine;hydrochloride |
| 0.0009772 | 0.001318 | 0.741426404 | 4 | 134152667 | N-(3,5-dimethoxyphenyl)-N-[3-(3-fluoropyrrolidin-1-yl)propyl]- |
| | | | | | 3-(1-methylpyrazol-4-yl)quinoxalin-6-amine;hydrochloride |
| 0.0009772 | 0.001318 | 0.741426404 | 3 | 134140921 | 2-(N-[3-[1-(3-aminopropyl)pyrazol-4-yl]quinoxalin-6-yl]-3,5- |
| | | | | | dimethoxyanilino)ethanol;hydrochloride |
| 0.00195 | 0.00263 | 0.741444867 | 3 | 68209345 | 3-fluoro-5-[2-hydroxyethyl-[3-(1-methylpyrazol-4-yl)quinoxalin- |
| | | | | | 6-yl]amino]-N-methylbenzamide |
| 0.012 | 0.016 | 0.75 | 3 | 67161103 | 5-[(1-ethylpiperidin-4-yl)amino]-3-[1H-imidazol-2- |
| | | | | | yl(phenyl)methylidene]-1H-indol-2-one |
| 0.008128 | 0.01072 | 0.758208955 | 4 | 134138098 | N′-(3,5-dimethoxyphenyl)-N-propan-2-yl-N′-[3-(1,3,5- |
| | | | | | trimethylpyrazol-4-yl)quinoxalin-6-yl]ethane-1,2- |
| | | | | | diamine;hydrochloride |
| 0.11482 | 0.15136 | 0.758588795 | 3 | 54591211 | N′-(2,5-dimethoxyphenyl)-N′-[3-(1-methylpyrazol-4- |
| | | | | | yl)quinoxalin-6-yl]-N-propan-2-ylethane-1,2-diamine |
| 0.0631 | 0.08318 | 0.758595816 | 4 | 134150399 | 2-(2,5-dimethoxy-N-[3-(1-methylpyrazol-4-yl)quinoxalin-6- |
| | | | | | yl]anilino)ethanol;hydrochloride |
| 0.002291 | 0.00302 | 0.758609272 | 4 | 68208778 | N-(cyclopropylmethyl)-N′-(3,5-dimethoxyphenyl)-N′-[3-(1- |
| | | | | | methylpyrazol-4-yl)quinoxalin-6-yl]ethane-1,2-diamine |
| 0.007943 | 0.01047 | 0.758643744 | 4 | 134142457 | N-[3-(3,3-difluoropyrrolidin-1-yl)propyl]-N-(3,5- |
| | | | | | dimethoxyphenyl)-3-(1H-pyrazol-4-yl)quinoxalin-6- |
| | | | | | amine;hydrochloride |
| 0.007943 | 0.01047 | 0.758643744 | 4 | 54591126 | N-[3-(3,3-difluoropyrrolidin-1-yl)propyl]-N-(3,5- |
| | | | | | dimethoxyphenyl)-3-(1H-pyrazol-4-yl)quinoxalin-6-amine |
| 0.00182 | 0.002399 | 0.758649437 | 4 | 68208767 | 1-[2-(3,5-dimethoxy-N-[3-(1-methylpyrazol-4-yl)quinoxalin-6- |
| | | | | | yl]anilino)ethyl]piperidine-3-carboxamide |
| 0.000912 | 0.001202 | 0.758735441 | 3 | 134150157 | 3-methoxy-5-[2-(propan-2-ylamino)ethyl-[3-(1H-pyrazol-4- |
| | | | | | yl)quinoxalin-6-yl]amino]phenol;hydrochloride |
| 0.002692 | 0.003548 | 0.758737317 | 4 | 89447204 | N-(3,5-dimethoxyphenyl)-N-[3-[(2S,6R)-2,6-dimethylmorpholin- |
| | | | | | 4-yl]propyl]-3-(1-methylpyrazol-4-yl)quinoxalin-6-amine |
| 0.03311 | 0.04266 | 0.776136896 | 4 | 67466417 | N-[3-(6-aminopyridin-2-yl)prop-2-ynyl]-N-(3,5- |
| | | | | | dimethoxyphenyl)-3-(1-methylpyrazol-4-yl)quinoxalin-6-amine |
| 0.00138 | 0.001778 | 0.776152981 | 4 | 68209446 | N-(3,5-dimethoxyphenyl)-3-[1-(2-methylsulfonylethyl)pyrazol- |
| | | | | | 4-yl]-N-(3-pyrrolidin-1-ylpropyl)quinoxalin-6-amine |
| 0.001259 | 0.001622 | 0.776202219 | 4 | 89447256 | N-(3,5-dimethoxyphenyl)-3-(1-methylpyrazol-4-yl)-N-[3-(3- |
| | | | | | methylsulfonylpyrrolidin-1-yl)propyl]quinoxalin-6-amine |
| 0.0005623 | 0.0007244 | 0.776228603 | 4 | 68208755 | 7-[N-[2-(dimethylsulfamoylamino)ethyl]-3,5-dimethoxyanilino]- |
| | | | | | 2-(1-methylpyrazol-4-yl)quinoxaline |
| 0.001738 | 0.002239 | 0.776239393 | 4 | 68209464 | 2-[2-(3,5-dimethoxy-N-[3-(1-methylpyrazol-4-yl)quinoxalin-6- |
| | | | | | yl]anilino)ethylamino]ethanol |
| 0.002884 | 0.003715 | 0.776312248 | 4 | 134153603 | N′-(2-chloro-5-methoxyphenyl)-N′-[3-(1-methylpyrazol-4- |
| | | | | | yl)quinoxalin-6-yl]-N-propan-2-ylethane-1,2- |
| | | | | | diamine;hydrochloride |
| 0.001549 | 0.001995 | 0.776441103 | 4 | 134145938 | N-(3,5-dimethoxyphenyl)-3-(1-methylpyrazol-4-yl)-N-[2-[(2R)- |
| | | | | | 2-methylpyrrolidin-1-yl]ethyl]quinoxalin-6-amine;hydrochloride |
| 0.014 | 0.018 | 0.777777778 | 7 | 11847343 | 3-[(4-morpholin-4-ylbenzoyl)amino]-N-[(1S)-1-phenyl-2- |
| | | | | | pyrrolidin-1-ylethyl]-1H-thieno[5,4-d]pyrazole-5-carboxamide |
| 0.6 | 0.76 | 0.789473684 | 4 | 24949856 | Hematoxylene |
| 0.001698 | 0.002138 | 0.794200187 | 4 | 89447335 | N-[2-(3,5-dimethoxy-N-[3-(1-methylpyrazol-4-yl)quinoxalin-6- |
| | | | | | yl]anilino)ethyl]-3,3,3-trifluoro-2-hydroxy-2-methylpropanamide |
| 0.002138 | 0.002692 | 0.794205052 | 4 | 68209337 | N′-(3-fluoro-5-methoxyphenyl)-N′-[3-(1-methylpyrazol-4- |
| | | | | | yl)quinoxalin-6-yl]-N-propan-2-ylethane-1,2-diamine |
| 0.01 | 0.01259 | 0.794281176 | 4 | 89447494 | N′-(3,5-dimethoxyphenyl)-N′-[3-(5-ethyl-1-methylpyrazol-4- |
| | | | | | yl)quinoxalin-6-yl]-N-(2,2,2-trifluoroethyl)ethane-1,2-diamine |
| 0.01259 | 0.01585 | 0.794321767 | 4 | 89447345 | N-[3-(3-chloropyrazin-2-yl)prop-2-ynyl]-N-(3,5- |
| | | | | | dimethoxyphenyl)-3-(1-methylpyrazol-4-yl)quinoxalin-6-amine |
| 0.1349 | 0.16982 | 0.79437051 | 4 | 68208915 | N-(3,5-dimethoxyphenyl)-N-[3-(3-methoxyphenyl)prop-2-ynyl]- |
| | | | | | 3-(1-methylpyrazol-4-yl)quinoxalin-6-amine |
| 0.002399 | 0.00302 | 0.794370861 | 4 | 134131759 | N′-(3,5-dimethoxyphenyl)-N′-[3-(1H-pyrazol-4-yl)quinoxalin-6- |
| | | | | | yl]ethane-1,2-diamine;hydrochloride |
| 0.00166 | 0.002089 | 0.794638583 | 4 | 134153940 | N′-(3-fluoro-5-methoxyphenyl)-N-propan-2-yl-N′-[3-(1H- |
| | | | | | pyrazol-4-yl)quinoxalin-6-yl]ethane-1,2-diamine;hydrochloride |
| 0.00166 | 0.002089 | 0.794638583 | 3 | 68208724 | 4-[[4-[7-[N-(cyclopropylmethyl)-3,5- |
| | | | | | dimethoxyanilino]quinoxalin-2-yl]pyrazol-1-yl]methyl]piperidin- |
| | | | | | 4-ol |
| 0.00263 | 0.003236 | 0.812731768 | 4 | 68209138 | N-(2,2-difluoropropyl)-N′-(3,5-dimethoxyphenyl)-N′-[3-(1- |
| | | | | | methylpyrazol-4-yl)quinoxalin-6-yl]propane-1,3-diamine |
| 0.001862 | 0.002291 | 0.812745526 | 3 | 68208700 | N-(cyclopropylmethyl)-N-(3,5-dimethoxyphenyl)-3-[1- |
| | | | | | (piperidin-4-ylmethyl)pyrazol-4-yl]quinoxalin-6-amine |
| 0.005623 | 0.006918 | 0.81280717 | 5 | 68209431 | 3-methoxy-N-methyl-5-[3-(1-methylpyrazol-4-yl)quinoxalin-6- |
| | | | | | yl]-[2-(propan-2-ylamino)ethyl]amino]benzamide |
| 0.002042 | 0.002512 | 0.812898089 | 4 | 134135888 | N-(3,5-dimethoxyphenyl)-N-[3-[(2R,5S)-2,5-dimethylpyrrolidin- |
| | | | | | 1-yl]propyl]-3-(1-methylpyrazol-4-yl)quinoxalin-6- |
| | | | | | amine;hydrochloride |
| 0.04467 | 0.05495 | 0.812920837 | 4 | 68209404 | N-(cyclopentylmethyl)-N-(3,5-dimethoxyphenyl)-3-(1- |
| | | | | | methylpyrazol-4-yl)quinoxalin-6-amine |
| 0.008913 | 0.01072 | 0.831436567 | 4 | 68208924 | N-(3,5-dimethoxyphenyl)-3-(1-methylpyrazol-4-yl)-N-[3-(4- |
| | | | | | methylpyridin-2-yl)propyl]quinoxalin-6-amine |
| 0.01259 | 0.01514 | 0.831571995 | 5 | 68209322 | N′-(3,5-dimethoxyphenyl)-N′-[3-[5-[(dimethylamino)methyl]-1- |
| | | | | | methylpyrazol-4-yl]quinoxalin-6-yl]-N-propan-2-ylethane-1,2- |
| | | | | | diamine |
| 0.001047 | 0.001259 | 0.831612391 | 4 | 68209010 | (2R)-3-(N-[3-[1-(3-aminopropyl)pyrazol-4-yl]quinoxalin-6-yl]- |
| | | | | | 3,5-dimethoxyanilino)-1,1, 1-trifluoropropan-2-ol |
| 0.005888 | 0.007079 | 0.831755898 | 3 | 89447547 | 4-[3-(3,5-dimethoxy-N-[3-(1-methylpyrazol-4-yl)quinoxalin-6- |
| | | | | | yl]anilino)prop-1-ynyl]piperidin-4-ol |
| 0.002818 | 0.003388 | 0.83175915 | 4 | 68209310 | ethyl 1-[2-(3,5-dimethoxy-N-[3-(1-methylpyrazol-4- |
| | | | | | yl)quinoxalin-6-yl]anilino)ethyl]piperidine-4-carboxylate |
| 0.00309 | 0.003715 | 0.831763122 | 4 | 68208740 | N-(3,5-dimethoxyphenyl)-3-(1-methylpyrazol-4-yl)-N-(thiophen- |
| | | | | | 2-ylmethyl)quinoxalin-6-amine |
| 0.006607 | 0.007943 | 0.831801586 | 4 | 89447313 | N-(3,5-dimethoxyphenyl)-3-(1-methylpyrazol-4-yl)-N-[3-(1,3- |
| | | | | | thiazol-2-yl)prop-2-ynyl]quinoxalin-6-amine |
| 0.001202 | 0.001445 | 0.83183391 | 4 | 68209075 | N′-(3,5-dimethoxyphenyl)-N′-[3-[1-(2- |
| | | | | | methylsulfonylethyl)pyrazol-4-yl]quinoxalin-6-yl]propane-1,3- |
| | | | | | diamine |
| 0.006026 | 0.007244 | 0.83186085 | 4 | 134138612 | N′-(3-methoxyphenyl)-N′-[3-(1-methylpyrazol-4-yl)quinoxalin- |
| | | | | | 6-yl]-N-propan-2-ylethane-1,2-diamine;hydrochloride |
| 0.001514 | 0.00182 | 0.831868132 | 4 | 134154939 | 1-[2-(3,5-dimethoxy-N-[3-(1-methylpyrazol-4-yl)quinoxalin-6- |
| | | | | | yl]anilino)ethyl]pyrrolidin-3-ol;hydrochloride |
| 0.002692 | 0.003236 | 0.831891224 | 4 | 89447423 | N-(3,5-dimethoxyphenyl)-N-[3-[(2R,5S)-2,5-dimethylpyrrolidin- |
| | | | | | 1-yl]propyl]-3-(1-methylpyrazol-4-yl)quinoxalin-6-amine |
| 0.00195 | 0.002344 | 0.831911263 | 4 | 89447091 | (2S)-3-(N-[3-[1-(3-aminopropyl)pyrazol-4-yl]quinoxalin-6-yl]- |
| | | | | | 3,5-dimethoxyanilino)-1,1, 1-trifluoropropan-2-ol |
| 0.002455 | 0.002951 | 0.831921383 | 4 | 68209482 | 1-(3,5-dimethoxy-N-[3-(1-methylpyrazol-4-yl)quinoxalin-6- |
| | | | | | yl]anilino)-3-piperidin-1-ylpropan-2-ol |
| 0.11 | 0.13 | 0.846153846 | 10 | 5328112 | 1-(2-Amino-6-(2,6-dichlorophenyl)pyrido[2,3-d]pyrimidin-7-yl)- |
| | | | | | 3-(tert-butyl)urea |
| 0.002089 | 0.002455 | 0.850916497 | 4 | 89447314 | N-(3,5-dimethoxyphenyl)-N-[(2-methyl-1,3-thiazol-4- |
| | | | | | yl)methyl]-3-(1H-pyrazol-4-yl)quinoxalin-6-amine |
| 0.002291 | 0.002692 | 0.851040119 | 4 | 68209083 | N′-(3,5-dimethoxyphenyl)-N′-[3-(1-methylpyrazol-4- |
| | | | | | yl)quinoxalin-6-yl]butane-1,4-diamine |
| 0.007586 | 0.008913 | 0.851116347 | 4 | 68208680 | N-(3,5-dimethoxyphenyl)-N-[(1-methylimidazol-2-yl)methyl]-3- |
| | | | | | (1-methylpyrazol-4-yl)quinoxalin-6-amine |
| 0.002399 | 0.002818 | 0.851312988 | 4 | 134149546 | 1-(3,5-dimethoxy-N-[3-(1-methylpyrazol-4-yl)quinoxalin-6- |
| | | | | | yl]anilino)-3-(methylamino)propan-2-ol;hydrochloride |
| 0.01096 | 0.01259 | 0.870532168 | 4 | 68209408 | N-[(5-chlorothiophen-2-yl)methyl]-N-(3,5-dimethoxyphenyl)-3- |
| | | | | | (1-methylpyrazol-4-yl)quinoxalin-6-amine |
| 0.00138 | 0.001585 | 0.870662461 | 5 | 88888594 | 2-(2,6-difluoro-3,5-dimethoxy-N-[3-(1-methylpyrazol-4- |
| | | | | | yl)quinoxalin-6-yl]anilino)ethanol |
| 0.005754 | 0.006607 | 0.870894506 | 4 | 134141925 | N-[3-(3,3-difluoropyrrolidin-1-yl)propyl]-N-(3,5- |
| | | | | | dimethoxyphenyl)-3-[1-(oxan-4-ylmethyl)pyrazol-4- |
| | | | | | yl]quinoxalin-6-amine;hydrochloride |
| 0.00302 | 0.003467 | 0.871070089 | 4 | 68209101 | 3-[2-aminoethyl-[3-(1-methylpyrazol-4-yl)quinoxalin-6- |
| | | | | | yl]amino]-5-methoxy-N-methylbenzamide |
| 0.01549 | 0.01778 | 0.8712036 | 4 | 89447472 | N-(3,5-dimethoxyphenyl)-3-(1-methylpyrazol-4-yl)-N-(3- |
| | | | | | pyrazin-2-ylprop-2-ynyl)quinoxalin-6-amine |
| 0.003388 | 0.003802 | 0.891109942 | 4 | 134155253 | (2S)-1-[2-(3,5-dimethoxy-N-[3-(1-methylpyrazol-4- |
| | | | | | yl)quinoxalin-6-yl]anilino)ethyl]pyrrolidine-2-carboxylic |
| | | | | | acid;hydrochloride |
| 0.03388 | 0.03802 | 0.891109942 | 3 | 134157453 | N-(2,5-dimethoxyphenyl)-3-(1-methylpyrazol-4-yl)-N-(3- |
| | | | | | pyrrolidin-1-ylpropyl)quinoxalin-6-amine;hydrochloride |
| 0.005623 | 0.00631 | 0.891125198 | 5 | 134138981 | N′-(3,5-dimethoxyphenyl)-N-methyl-N′-[3-[1-(oxan-4- |
| | | | | | ylmethyl)pyrazol-4-yl]quinoxalin-6-yl]ethane-1,2- |
| | | | | | diamine;hydrochloride |
| 0.005754 | 0.006457 | 0.89112591 | 3 | 68209303 | 3-[[3-[1-(2-hydroxyethyl)pyrazol-4-yl]quinoxalin-6-yl]-(3- |
| | | | | | pyrrolidin-1-ylpropyl)amino]-5-methoxy-N-methylbenzamide |
| 0.007943 | 0.008913 | 0.891170201 | 4 | 89447373 | 3-Methoxy-5-[[3-(1-methylpyrazol-4-yl)quinoxalin-6-yl]-[3- |
| | | | | | (2,2,2-trifluoroethylamino)propyl]amino]phenol |
| 0.004571 | 0.005129 | 0.891206863 | 3 | 68209057 | N-(3,5-dimethoxyphenyl)-N-[2-[4-(dimethylamino)piperidin-1- |
| | | | | | yl]ethyl]-3-(1-methylpyrazol-4-yl)quinoxalin-6-amine |
| 0.22387 | 0.25119 | 0.891237709 | 4 | 68209503 | N-(3,5-dimethoxyphenyl)-3-(1-methylpyrazol-4-yl)-N-[3-(5- |
| | | | | | methylpyridin-2-yl)prop-2-ynyl]quinoxalin-6-amine |
| 0.008318 | 0.009333 | 0.891246116 | 4 | 89447538 | N-(3,5-dimethoxyphenyl)-3-(1-methylpyrazol-4-yl)-N-(3- |
| | | | | | pyrimidin-2-ylprop-2-ynyl)quinoxalin-6-amine |
| 0.003467 | 0.00389 | 0.89125964 | 4 | 68209085 | 2-[1-[2-(3,5-dimethoxy-N-[3-(1-methylpyrazol-4-yl)quinoxalin- |
| | | | | | 6-yl]anilino)ethyl]piperidin-4-yl]propan-2-ol |
| 0.002692 | 0.00302 | 0.891390728 | 4 | 134141393 | N-(3,5-dimethoxyphenyl)-3-(1-methylpyrazol-4-yl)-N- |
| | | | | | (piperidin-4-ylmethyl)quinoxalin-6-amine;hydrochloride |
| 0.001175 | 0.001318 | 0.891502276 | 4 | 134151087 | N′-(3,5-dimethoxyphenyl)-N′-[3-[1-(oxan-4-ylmethyl)pyrazol-4- |
| | | | | | yl]quinoxalin-6-yl]ethane-1,2-diamine;hydrochloride |
| 0.71 | 0.78 | 0.91025641 | 5 | 5328125 | 1-[2-Amino-6-(2,3,5,6-tetramethylphenyl)pyrido[2,3- |
| | | | | | d]pyrimidin-7-yl]-3-tert-butylurea |
| 0.001778 | 0.00195 | 0.911794872 | 4 | 68208862 | N′-(3,5-dimethoxyphenyl)-N′-[3-(1-propan-2-ylpyrazol-4- |
| | | | | | yl)quinoxalin-6-yl]ethane-1,2-diamine |
| 0.01096 | 0.01202 | 0.911813644 | 4 | 68208774 | N′-(3,5-dichlorophenyl)-N′-[3-(1-methylpyrazol-4-yl)quinoxalin- |
| | | | | | 6-yl]-N-propan-2-ylethane-1,2-diamine |
| 0.001585 | 0.001738 | 0.911967779 | 4 | 88888612 | N-(2,2-difluoroethyl)-N′-(3,5-dimethoxyphenyl)-N′-[3-(1- |
| | | | | | methylpyrazol-4-yl)quinoxalin-6-yl]propane-1,3-diamine |
| 0.007762 | 0.008511 | 0.91199624 | 5 | 89447553 | 4-[3-(3,5-dimethoxy-N-[3-(1-methylpyrazol-4-yl)quinoxalin-6- |
| | | | | | yl]anilino)prop-1-ynyl]pyridine-3-carbonitrile |
| 0.002188 | 0.002399 | 0.912046686 | 4 | 67461665 | N′-(3,5-dimethoxyphenyl)-N′-[3-(1-methylpyrazol-4- |
| | | | | | yl)quinoxalin-6-yl]ethane-1,2-diamine |
| 0.002399 | 0.00263 | 0.9121673 | 4 | 68209340 | 1-(3,5-dimethoxy-N-[3-(1-methylpyrazol-4-yl)quinoxalin-6- |
| | | | | | yl]anilino)-3-(methylamino)propan-2-ol |
| 0.001738 | 0.001905 | 0.912335958 | 4 | 134151320 | N-(3,5-dimethoxyphenyl)-N-[3-(2,2-dimethylpyrrolidin-1- |
| | | | | | yl)propyl]-3-(1-methylpyrazol-4-yl)quinoxalin-6- |
| | | | | | amine;hydrochloride |
| 0.003715 | 0.003981 | 0.933182617 | 4 | 68209445 | N′-(3,5-dimethoxyphenyl)-N-methyl-N′-[3-(1H-pyrazol-4- |
| | | | | | yl)quinoxalin-6-yl]ethane-1,2-diamine |
| 0.003548 | 0.003802 | 0.933193056 | 4 | 134136731 | N′-[3-[1-(3-aminopropyl)pyrazol-4-yl]quinoxalin-6-yl]-N′-(3,5- |
| | | | | | dimethoxyphenyl)-N-(2,2,2-trifluoroethyl)propane-1,3- |
| | | | | | diamine;hydrochloride |
| 0.008511 | 0.00912 | 0.933223684 | 4 | 134144581 | N-(3,5-dimethoxyphenyl)-N-[2-[(2S,6R)-2,6-dimethylmorpholin- |
| | | | | | 4-yl]ethyl]-3-(1-methylpyrazol-4-yl)quinoxalin-6- |
| | | | | | amine;hydrochloride |
| 0.003467 | 0.003715 | 0.933243607 | 4 | 88888575 | N-(3,5-dimethoxyphenyl)-3-(1-methylpyrazol-4-yl)-N-(oxiran-2- |
| | | | | | ylmethyl)quinoxalin-6-amine |
| 0.007244 | 0.007762 | 0.933264623 | 3 | 89447346 | cyclopropyl-[4-[2-(3,5-dimethoxy-N-[3-(1-methylpyrazol-4- |
| | | | | | yl)quinoxalin-6-yl]anilino)ethyl]piperazin-1-yl]methanone |
| 0.01862 | 0.01995 | 0.933333333 | 5 | 89446990 | tert-butyl (1S,4S)-5-[2-(3,5-dimethoxy-N-[3-(1-methylpyrazol-4- |
| | | | | | yl)quinoxalin-6-yl]anilino)ethyl]-2,5-diazabicyclo[2.2.1]heptane- |
| | | | | | 2-carboxylate |
| 0.029 | 0.031 | 0.935483871 | 7 | 5328158 | 6-Arylpyrido[2,3-d]pyrimidine deriv. 56 |
| 0.003467 | 0.003631 | 0.954833379 | 4 | 89447231 | N′-(3,5-dimethoxyphenyl)-N-methyl-N′-[3-[1-(2- |
| | | | | | methylsulfonylethyl)pyrazol-4-yl]quinoxalin-6-yl]ethane-1,2- |
| | | | | | diamine |
| 0.003388 | 0.003548 | 0.954904171 | 3 | 68208671 | N-[2-(3,5-dimethoxy-N-[3-(1-methylpyrazol-4-yl)quinoxalin-6- |
| | | | | | yl]anilino)ethyl]-1-methylpiperidine-3-carboxamide |
| 0.007079 | 0.007413 | 0.954944017 | 4 | 68208970 | 2-[4-[7-[3,5-dimethoxy-N-[2- |
| | | | | | (methylamino)ethyl]anilino]quinoxalin-2-yl]pyrazol-1-yl]ethanol |
| 0.006918 | 0.007244 | 0.954997239 | 4 | 89447342 | (NZ)-N-[1-(3,5-dimethoxy-N-[3-(1-methylpyrazol-4- |
| | | | | | yl)quinoxalin-6-yl]anilino)-3-methylbutan-2- |
| | | | | | ylidene]hydroxylamine |
| 0.008913 | 0.009333 | 0.954998393 | 4 | 67466880 | N-(3,5-dimethoxyphenyl)-3-(1-methylpyrazol-4-yl)-N-[3-(4- |
| | | | | | methylpyridin-2-yl)prop-2-ynyl]quinoxalin-6-amine |
| 0.01047 | 0.01072 | 0.976679104 | 4 | 67466492 | N-(3,5-dimethoxyphenyl)-3-(1-methylpyrazol-4-yl)-N-(3- |
| | | | | | pyridin-2-ylprop-2-ynyl)quinoxalin-6-amine |
| 0.003715 | 0.003802 | 0.977117307 | 4 | 89447247 | N-(3,5-dimethoxyphenyl)-N-[2-[(2S,5S)-2,5-dimethylpyrrolidin- |
| | | | | | 1-yl]ethyl]-3-(1-methylpyrazol-4-yl)quinoxalin-6-amine |
| 0.002951 | 0.00302 | 0.977152318 | 4 | 54591130 | N-(3,5-dimethoxyphenyl)-3-(1-methylpyrazol-4-yl)-N-[2-(2-oxa- |
| | | | | | 6-azaspiro[3.3]heptan-6-yl)ethyl]quinoxalin-6-amine |
| 0.0007586 | 0.0007762 | 0.977325432 | 4 | 134157956 | N-[3-(3,5-dimethoxy-N-[3-(1H-pyrazol-4-yl)quinoxalin-6- |
| | | | | | yl]anilino)propyl]-2,2,2-trifluoroacetamide;2,2,2-trifluoroacetic |
| | | | | | acid |
| 0.03631 | 0.03715 | 0.977388964 | 4 | 134139173 | 1-N-(3,5-dimethoxyphenyl)-3-methyl-1-N-[3-(1-methylpyrazol- |
| | | | | | 4-yl)quinoxalin-6-yl]-2-N-propan-2-ylbutane-1,2- |
| | | | | | diamine;hydrochloride |
| 0.001738 | 0.001778 | 0.977502812 | 4 | 67466509 | 1-(3,5-dimethoxy-N-[3-(1-propan-2-ylpyrazol-4-yl)quinoxalin-6- |
| | | | | | yl]anilino)-3-methoxypropan-2-ol |
| 0.001738 | 0.001778 | 0.977502812 | 4 | 68209125 | (2S)-1-(3,5-dimethoxy-N-[3-(1-methylpyrazol-4-yl)quinoxalin-6- |
| | | | | | yl]anilino)-3-(methylamino)propan-2-ol |
| 0.001738 | 0.001778 | 0.977502812 | 4 | 134157525 | 1-(3,5-dimethoxy-N-[3-(1-propan-2-ylpyrazol-4-yl)quinoxalin-6- |
| | | | | | yl]anilino)-3-methoxypropan-2-ol;hydrochloride |
| 3.7 | 3.71 | 0.997304582 | 11 | 5329111 | 1-(2-Amino-6-phenylpyrido[2,3-d]pyrimidin-7-yl)-3-tert- |
| | | | | | butylurea |
| 0.001778 | 0.001778 | 1 | 4 | 68208757 | N-(3,5-dimethoxyphenyl)-3-(1-methylpyrazol-4-yl)-N-[(2- |
| | | | | | methyl-1,3-thiazol-4-yl)methyl]quinoxalin-6-amine |
| 0.001905 | 0.001905 | 1 | 4 | 134135121 | (2S)-1-(3,5-dimethoxy-N-[3-(1-methylpyrazol-4-yl)quinoxalin-6- |
| | | | | | yl]anilino)-3-(propan-2-ylamino)propan-2-ol;hydrochloride |
| 0.003 | 0.003 | 1 | 5 | 5330135 | 1,6-Naphthyridine 25 |
| 0.033 | 0.033 | 1 | 5 | 5330168 | Pyrido[2,3-d]pyrimidine 104 |
| 0.0036 | 0.0036 | 1 | 4 | 5330126 | 1,6-Naphthyridine 16 |
| 0.001 | 0.001 | 1 | 5 | 5330127 | 1,6-Naphthyridine 17 |
| 0.002 | 0.002 | 1 | 5 | 5330128 | 1,6-Naphthyridine 18 |
| 0.003 | 0.003 | 1 | 5 | 5330129 | 1,6-Naphthyridine 19 |
| 0.0004 | 0.0004 | 1 | 5 | 5330130 | 1,6-Naphthyridine 20 |
| 0.0026 | 0.0026 | 1 | 4 | 5330132 | 1,6-Naphthyridine 22 |
| 0.02 | 0.02 | 1 | 5 | 5330169 | Pyrido[2,3-d]pyrimidine 105 |
| 0.0005 | 0.0005 | 1 | 4 | 5330133 | 1,6-Naphthyridine 23 |
| 0.002 | 0.002 | 1 | 5 | 5330136 | 1,6-Naphthyridine 26 |
| 0.0027 | 0.0027 | 1 | 5 | 5330157 | 1,6-Naphthyridine 75 |
| 0.0047 | 0.0047 | 1 | 7 | 5330158 | 1,6-Naphthyridine 76 |
| 0.003 | 0.003 | 1 | 5 | 5330159 | 1,6-Naphthyridine 77 |
| 0.015 | 0.015 | 1 | 5 | 5330166 | Pyrido[2,3-d]pyrimidine 102 |
| 0.006 | 0.006 | 1 | 5 | 5330167 | Pyrido[2,3-d]pyrimidine 103 |
| 0.0005 | 0.0005 | 1 | 4 | 5330134 | 1,6-Naphthyridine 24 |
| 0.001698 | 0.001698 | 1 | 4 | 54591291 | N-[3-(3,5-dimethoxy-N-[3-(1-methylpyrazol-4-yl)quinoxalin-6- |
| | | | | | yl]anilino)propyl]-3,3,3-trifluoro-2-hydroxy-2- |
| | | | | | methylpropanamide |
| 0.054 | 0.054 | 1 | 7 | 11464544 | 2,4-Difluoro-5-(5-isopropyl-6-[1,3,4]oxadiazol-2-yl-pyrrolo[2,1- |
| | | | | | f][1,2,4]triazin-4-ylamino)-N-methoxy-benzamide |
| 0.0002 | 0.0002 | 1 | 3 | 127047969 | 7-(1,3,4,6,7,8,9,9a-octahydropyrido[1,2-a]pyrazin-2-yl)-N-[5-[2- |
| | | | | | (3,5-dimethoxyphenyl)ethyl]-1H-pyrazol-3-yl]quinazolin-4- |
| | | | | | amine |
| 6.8 | 6.8 | 1 | 5 | 44330158 | 7-Oxo-5-phenyl-7H-[2]pyrindine-6-carboxylic acid methyl ester |
| 6.4 | 6.4 | 1 | 5 | 44330102 | Methyl 5-methyl-1-oxo-3-phenylindene-2-carboxylate;methyl 5- |
| | | | | | methyl-3-oxo-1-phenylindene-2-carboxylate |
| 5.1 | 5.1 | 1 | 5 | 10015651 | 1-Oxo-3-phenyl-1H-indene-2-carboxylic acid methyl ester |
| 0.001 | 0.001 | 1 | 3 | 135819982 | 1-tert-butyl-3-(3-(5-(4-(piperidin-1-yl)piperidin-1-yl)-1H- |
| | | | | | benzo[d]imidazol-2-yl)-1H-indazol-5-yl)urea |
| 0.001 | 0.001 | 1 | 5 | 5329004 | 1,6-Naphthyridine deriv. 20 |
| 5 | 5 | 1 | 9 | 137631210 | 3-[(3S)-3-aminopyrrolidine-1-carbonyl]-5,10-dihydroxy-2- |
| | | | | | methylnaphtho[2,3-f][1]benzofuran-4,11-dione;methanesulfonic |
| | | | | | acid |
| 5.4 | 5.4 | 1 | 5 | 44330203 | 3-(4-Chloro-phenyl)-1-oxo-1H-indene-2-carboxylic acid amide |
| 0.126 | 0.126 | 1 | 4 | 44294299 | 2-Amino-6-(3,5-dimethoxyphenyl)-8-methylpyrido[2,3- |
| | | | | | d]pyrimidine-7(8H)-one |
| 0.006166 | 0.006166 | 1 | 4 | 68208839 | N-[(4-benzylmorpholin-2-yl)methyl]-N-(3,5-dimethoxyphenyl)- |
| | | | | | 3-(1-methylpyrazol-4-yl)quinoxalin-6-amine |
| 0.14 | 0.14 | 1 | 4 | 10602350 | 1-[2-[[3-(4-Methylpiperazin-1-yl)propyl]amino]-6-(2,3,5,6- |
| | | | | | tetramethylphenyl)pyrido[2,3-d]pyrimidin-7-yl]-3-tert-butylurea |
| 0.001698 | 0.001698 | 1 | 4 | 134148391 | N-[3-(3,5-dimethoxy-N-[3-(1-methylpyrazol-4-yl)quinoxalin-6- |
| | | | | | yl]anilino)propyl]-3,3,3-trifluoro-2-hydroxy-2- |
| | | | | | methylpropanamide;hydrochloride |
| 0.006166 | 0.006166 | 1 | 4 | 118014724 | N-[3,5-bis(trideuteriomethoxy)phenyl]-3-(1-propan-2-ylpyrazol- |
| | | | | | 4-yl)-N-(3-pyridin-4-ylprop-2-ynyl)quinoxalin-6-amine |
| 0.046 | 0.046 | 1 | 7 | 11328182 | 2,4-Difluoro-5-[5-isopropyl-6-(5-methyl-4H-[1,2,4]triazol-3-yl)- |
| | | | | | pyrrolo[2,1-f][1,2,4]triazin-4-ylamino]-N-methoxy-benzamide |
| 0.001 | 0.001 | 1 | 4 | 11442803 | 2,4-Difluoro-5-[5-isopropyl-6-(5-isopropyl-[1,3,4]oxadiazol-2- |
| | | | | | yl)-pyrrolo[2,1-f][1,2,4]triazin-4-ylamino]-N-methoxy- |
| | | | | | benzamide |
| 0.033 | 0.033 | 1 | 7 | 11488852 | 2,4-Difluoro-5-[5-isopropyl-6-(5-trifluoromethyl- |
| | | | | | [1,3,4]oxadiazol-2-yl)-pyrrolo[2,1-f][1,2,4]triazin-4-ylamino]-N- |
| | | | | | methoxy-benzamide |
| 0.046 | 0.046 | 1 | 4 | 11477626 | [4-(2,4-Difluoro-5-methoxycarbamoyl-phenylamino)-5- |
| | | | | | isopropyl-pyrrolo[2,1-f][1,2,4]triazin-6-yl]-carbamic acid |
| | | | | | tetrahydro-furan-2-ylmethyl ester |
| 0.0065 | 0.0065 | 1 | 7 | 10478569 | 2,4-Difluoro-5-[5-isopropyl-6-(5-methyl-[1,3,4]oxadiazol-2-yl)- |
| | | | | | pyrrolo[2,1-f][1,2,4]triazin-4-ylamino]-N-methoxy-benzamide |
| 0.001 | 0.001 | 1 | 2 | 73707531 | N-(4-((3-(2,6-dichloro-3,5-dimethoxyphenyl)-1-(6-((4-(4- |
| | | | | | methylpiperazin-1-yl)phenyl)amino)pyrimidin-4- |
| | | | | | yl)ureido)methyl)phenyl)acrylamide |

### Column Key

FGFR1 IC50_μm: reported IC₅₀ to FGFR1 in μM.

Min IC50_μm: next lowest IC₅₀ value to any gene target in μM.

Relative IC50: IC₅₀ of target of interest divided by next lowest IC₅₀

Num low IC50 Targets: the number of gene targets that have a reported IC₅₀ within 10-fold of the IC₅₀ to the target of interest (VEGFR2 or FGFR1).

Pubchem_cid: Pubchem chemical ID

Compound name: Name of compound

TABLE 3
List of selective inhibitors of VEGFR2

| | Min | | Numlow | | |
|---|---|---|---|---|---|
| VEGFR2 | IC50_ | Relative | IC50 | Pubchem_ | |
| IC50_μM | μM | IC50 | Targets | CID | Compound Name |
| 0.0006 | 0.7 | 0.000857143 | 1 | 21956341 | 3-({4-[methyl(3-methyl-1H-indazol-6-yl)amino]pyrimidin-2- |
| | | | | | yl}amino)benzene-1-sulfonamide |
| 6.20E−05 | 0.058 | 0.001068966 | 1 | 9893493 | [4-(2,4-Difluoro-5-methoxycarbamoyl-phenylamino)-5-isopropyl- |
| | | | | | pyrrolo[2,1-f][1,2,4]triazin-6-yl]-carbamic acid 3-methanesulfonyl-propyl |
| | | | | | ester |
| 0.017 | 9 | 0.001888889 | 1 | 10273854 | 3-({4-[(1,2-dimethyl-1H-1,3-benzodiazol-5-yl)(methyl)amino]pyrimidin- |
| | | | | | 2-yl}amino)benzene-1-sulfonamide |
| 0.0013 | 0.68 | 0.001911765 | 1 | 24823094 | 6-(6,7-Dimethoxyquinazolin-4-yl)oxynaphthalene-1-carboxamide |
| 0.00037 | 0.185 | 0.002 | 1 | 11750694 | 4-[5-(Cyclopropylcarbamoyl)naphthalen-2-yl]oxy-7-methoxyquinoline-6- |
| | | | | | carboxamide |
| 3.30E−05 | 0.015 | 0.0022 | 1 | 15949613 | 1-{2-Fluoro-4-[(5-Methyl-5h-Pyrrolo[3,2-D]pyrimidin-4-Yl)oxy]phenyl}- |
| | | | | | 3-[3-(Trifluoromethyl)phenyl]urea |
| 0.0013 | 0.5 | 0.0026 | 1 | 56970263 | 5-[[(6S)-7-acetyl-6-methyl-6,8-dihydro-5H-pyrido[3,4-d]pyrimidin-4- |
| | | | | | yl]oxy]-N-(5-cyclopropyl-1,2-oxazol-3-yl)indole-1-carboxamide |
| 0.0076 | 2.9 | 0.00262069 | 1 | 24949548 | 3-({4-[methyl(2-methyl-2H-indazol-6-yl)amino]pyrimidin-2- |
| | | | | | yl}amino)benzene-1-sulfonamide |
| 0.0013 | 0.44 | 0.002954545 | 1 | 24824448 | |
| 0.0013 | 0.39 | 0.003333333 | 1 | 11211389 | 4-(5-Carbamoylnaphthalen-2-yl)oxy-7-methoxyquinoline-6-carboxamide |
| 0.0056 | 1.4 | 0.004 | 1 | 24949700 | 3-[[4-[(2,3-Dimethylindazol-6-yl)-methylamino]pyrimidin-2- |
| | | | | | yl]amino]benzenesulfonamide;dihydrochloride |
| 0.17 | 29.9 | 0.005685619 | 1 | 44408215 | 3-(5-(2,3-dihydrobenzo[b][1,4]dioxin-6-ylamino)-1,3,4-oxadiazol-2-yl)-N- |
| | | | | | (pyridin-4-ylmethyl)pyridin-2-amine |
| 0.002884 | 0.36 | 0.008011111 | 1 | 24823093 | N-cyclopropyl-6-(6,7-dimethoxyquinazolin-4-yl)oxynaphthalene-1- |
| | | | | | carboxamide |
| 0.0012 | 0.13 | 0.009230769 | 1 | 24823095 | 6-(7-Methoxyquinolin-4-yl)oxynaphthalene-1-carboxamide |
| 0.0008913 | 0.088 | 0.010128409 | 1 | 11474276 | 1-Naphthalenecarboxamide, 6-[(6,7-dimethoxy-4-quinolinyl)oxy]- |
| 0.0009 | 0.066 | 0.013636364 | 1 | 56970264 | N-[5-(1-methylcyclopropyl)-1H-pyrazol-3-yl]-5-[[(6S)-6-methyl-5,6,7,8- |
| | | | | | tetrahydropyrido[3,4-d]pyrimidin-4-yl]oxy]indole-1-carboxamide |
| 0.009 | 0.62 | 0.014516129 | 1 | 24822599 | N-cyclopentyl-6-(6,7-dimethoxyquinolin-4-yl)oxynaphthalene-1- |
| | | | | | carboxamide |
| 0.001 | 0.06 | 0.016666667 | 1 | 5329056 | 7-[[(3R)-1-Methyl-3-piperidinyl]methoxy]-6-methoxy-4-(2-fluoro-4- |
| | | | | | bromoanilino)quinazoline |
| 0.0006 | 0.027 | 0.022222222 | 1 | 11441375 | N-cyclopropyl-6-[(6,7-dimethoxyquinolin-4-yl)oxy]naphthalene-1- |
| | | | | | carboxamide |
| 0.001 | 0.045 | 0.022222222 | 1 | 11398307 | Benzamide, N-[6-[(6,7-dimethoxy-4-quinolinyl)oxy]-1-methyl-1H- |
| | | | | | indazol-3-yl]-3-(trifluoromethyl)- |
| 0.00116 | 0.052 | 0.022307692 | 1 | 44368815 | N-(3-Isoquinolin-4-yl-phenyl)-2-[(pyridin-4-ylmethyl)-amino]-benzamide |
| 0.005 | 0.19 | 0.026315789 | 1 | 24822600 | 6-(6,7-dimethoxyquinolin-4-yl)oxy-N-propan-2-ylnaphthalene-1- |
| | | | | | carboxamide |
| 0.063 | 2.36 | 0.026694915 | 1 | 5330388 | Pyrazine-Pyridine Biheteroaryl 39 |
| 3.50E−05 | 0.0013 | 0.026923077 | 1 | 25102847 | Cabozantinib |
| 0.001288 | 0.046 | 0.028 | 1 | 11486459 | 6-(6,7-dimethoxyquinolin-4-yl)oxy-N-ethylnaphthalene-1-carboxamide |
| 0.00019 | 0.0055 | 0.034545455 | 1 | 57505507 | N-[3-({2-[(Cyclopropylcarbonyl)amino]imidazo[1,2-B]pyridazin-6- |
| | | | | | Yl}oxy)phenyl]-1,3-Dimethyl-1h-Pyrazole-5-Carboxamide |
| 0.0038 | 0.11 | 0.034545455 | 1 | 11749561 | 6-(6, 7-dimethoxyquinolin-4-yl)oxy-N-methylnaphthalene-1-carboxamide |
| 0.036 | 1 | 0.036 | 1 | 24949545 | 3-[[4-[(1,3-Dimethylindazol-6-yl)-methylamino]pyrimidin-2- |
| | | | | | yl]amino]benzenesulfonamide;hydrochloride |
| 0.063 | 1.7 | 0.037058824 | 1 | 24949704 | 3-({4-[(1,2-dimethyl-1H-1,3-benzodiazol-6-yl)(methyl)amino]pyrimidin- |
| | | | | | 2-yl}amino)benzene-1-sulfonamide |
| 0.49 | 11 | 0.044545455 | 1 | 44406074 | N-cyclohexyl-3-phenyl-1H-thieno[2,3-c]pyrazole-5-carboxamide |
| 0.0012 | 0.023 | 0.052173913 | 1 | 25206346 | 2-Chloro-3-[(2-Cyanopropan-2-Yl)oxy]-N-{5-[{2- |
| | | | | | [(Cyclopropylcarbonyl)amino][1,3]thiazolo[5,4-B]pyridin-5- |
| | | | | | Yl}(Methyl)amino]-2-Fluorophenyl}benzamide |
| 0.0011 | 0.019 | 0.057894737 | 1 | 25127856 | N-[3-({2-[(cyclopropylcarbonyl)amino][1,2,4]triazolo[1,5-a]pyridin-6- |
| | | | | | yl}oxy)phenyl]-1,3-dimethyl-1H-pyrazole-5-carboxamide |
| 3.00E−05 | 0.0005 | 0.06 | 1 | 24757938 | N-(4-chlorophenyl)-6-[(6,7-dimethoxyquinolin-4-yl)oxy]naphthalene-1- |
| | | | | | carboxamide |
| 0.006 | 0.1 | 0.06 | 1 | 5329026 | Anilinoquinazoline deriv. 28 |
| 0.011 | 0.183 | 0.06010929 | 1 | 16742063 | (E)-1-(4-((6-amino-5-((methoxyimino)methyl)pyrimidin-4-yl)oxy)-2- |
| | | | | | chlorophenyl)-3-ethylurea |
| 0.005 | 0.08 | 0.0625 | 1 | 5329055 | 4-Anilinoquinazoline 29 |
| 0.035 | 0.55 | 0.063636364 | 1 | 135454992 | 4-(Benzothiazol-6-ylamino)-6-(1-methyl-1-phenyl-ethylamino)- |
| | | | | | [1,3,5]triazin-2-ol |
| 0.0012 | 0.018 | 0.066666667 | 1 | 6398883 | 2-(Pyridin-4-Ylmethylamino)-~{n}-[3- |
| | | | | | (Trifluoromethyl)phenyl]benzamide |
| 0.00026 | 0.0036 | 0.072222222 | 1 | 25123227 | N-[5-({2-[(cyclopropylcarbonyl)amino][1,2,4]triazolo[1,5-a]pyridin-6- |
| | | | | | yl}oxy)-2-fluorophenyl]-1,3-dimethyl-1H-pyrazole-5-carboxamide |
| 9.30E−05 | 0.0011 | 0.084545455 | 1 | 24767976 | Tak-593 |
| 0.022 | 0.233 | 0.094420601 | 1 | 11224593 | 7-Methoxy-4-[5-(2-methoxyethylcarbamoyl)naphthalen-2- |
| | | | | | yl]oxyquinoline-6-carboxamide |
| 0.04 | 0.42 | 0.095238095 | 1 | 10249989 | N-[4-(Benzothiazol-6-ylamino)-6-(1-methyl-1-phenyl-ethylamino)- |
| | | | | | [1,3,5]triazin-2-yl]-hydroxylamine |
| 0.01 | 0.1 | 0.1 | 3 | 5329046 | N-(4-bromo-2,6-difluorophenyl)-6-methoxy-7-[(1-methylpiperidin-4- |
| | | | | | yl)methoxy]quinazolin-4-amine |
| 0.006 | 0.06 | 0.1 | 3 | 5329053 | N-(4-bromo-2-fluorophenyl)-6-methoxy-7-[2-(1-methylpiperidin-4- |
| | | | | | yl)ethoxy]quinazolin-4-amine |
| 0.1 | 1 | 0.1 | 3 | 5329006 | N-(4-chloro-2-fluorophenyl)-6,7-dimethoxyquinazolin-4-amine |
| 0.1 | 1 | 0.1 | 12 | 24900378 | N-hydroxy-4-[4-[[4-[4-[(1R)-1-phenylethyl]amino]-7H-pyrrolo[2,3- |
| | | | | | d]pyrimidin-6-yl]phenyl]methyl]piperazin-1-yl]butanamide |
| 0.0005 | 0.0049 | 0.102040816 | 2 | 127037812 | 6-[2-[3-(methylsulfonylmethyl)anilino]pyrimidin-4-yl]oxy-N-pyridin-3- |
| | | | | | ylnaphthalene-1-carboxamide |
| 0.02 | 0.18 | 0.111111111 | 2 | 9797919 | N-(4-Chlorophenyl)-2-[(pyridin-4-ylmethyl)amino]benzamide |
| 0.073 | 0.64 | 0.1140625 | 2 | 16730105 | 2-(3-Hydroxy-4-methoxyanilino)-5,7-dihydro-6H-pyrimido[5,4-d]- |
| | | | | | [1]benzazepin-6-one |
| 0.023 | 0.2 | 0.115 | 2 | 11632737 | N-cyclopropyl-2,4-difluoro-5-((2-(pyridin-2-ylamino)thiazol-5- |
| | | | | | yl)methylamino)benzamide |
| 0.03 | 0.25 | 0.12 | 3 | 5329049 | N-(2-fluoro-4-methylphenyl)-6-methoxy-7-(piperidin-4- |
| | | | | | ylmethoxy)quinazolin-4-amine |
| 0.049 | 0.4 | 0.1225 | 2 | 135509841 | 4-(Benzothiazol-6-ylamino)-6-(benzyl-ethyl-amino)-[1,3,5]triazin-2-ol |
| 0.04 | 0.3 | 0.133333333 | 4 | 5329019 | Anilinoquinazoline deriv. 21 |
| 0.0007 | 0.0052 | 0.134615385 | 2 | 9823820 | Lenvatinib |
| 0.0046 | 0.03 | 0.153333333 | 2 | 11620410 | 4-({2-[(4-chloro-3-{[(2S)-1-methylpyrrolidin-2- |
| | | | | | yl]methoxy}phenyl)amino]-1,3-benzoxazol-5-yl}oxy)-N-methylpyridine- |
| | | | | | 2-carboxamide |
| 0.012 | 0.073 | 0.164383562 | 2 | 11362483 | 6-(6,7-dimethoxyquinolin-4-yl)oxy-N-(2-methoxyethyl)naphthalene-1- |
| | | | | | carboxamide |
| 0.0036 | 0.021 | 0.171428571 | 2 | 25128181 | N-[3-({2-[(cyclopropylcarbonyl)amino]-1,3-benzothiazol-6- |
| | | | | | yl}oxy)phenyl]-1,3-dimethyl-1H-pyrazole-5-carboxamide |
| 0.007 | 0.04 | 0.175 | 3 | 5329011 | N-(4-chloro-2-fluorophenyl)-6-methoxy-7-(2-methoxyethoxy)quinazolin- |
| | | | | | 4-amine |
| 0.098 | 0.545 | 0.179816514 | 12 | 127027714 | 4-[4-(4-bromoanilino)-7-methoxyquinazolin-6-yl]oxy-N- |
| | | | | | hydroxybutanamide |
| 0.009 | 0.05 | 0.18 | 3 | 5329020 | Anilinoquinazoline deriv. 22 |
| 0.01 | 0.054 | 0.185185185 | 3 | 151194 | Vatalanib |
| 0.0011 | 0.0059 | 0.186440678 | 3 | 25127854 | N-[3-({2-[(cyclopropylcarbonyl)amino]imidazo[1,2-a]pyridin-6- |
| | | | | | yl}oxy)phenyl]-1,3-dimethyl-1H-pyrazole-5-carboxamide |
| 0.0009 | 0.0046 | 0.195652174 | 2 | 86302541 | 6-(4-aminothieno[2,3-d]pyrimidin-5-yl)-N-phenylnaphthalene-1- |
| | | | | | carboxamide |
| 0.016 | 0.077 | 0.207792208 | 2 | 151193 | Vatalanib succinate |
| 0.015 | 0.07 | 0.214285714 | 3 | 5329051 | N-(4-bromo-2,6-difluorophenyl)-6-methoxy-7-(piperidin-4- |
| | | | | | ylmethoxy)quinazolin-4-amine |
| 0.036 | 0.167 | 0.215568862 | 3 | 25094210 | 3-methyl-N-[6-methylsulfanyl-3-(1H-pyrazol-4-yl)imidazo[1,2-a]pyrazin- |
| | | | | | 8-yl]-1,2-thiazol-5-amine |
| 0.0007 | 0.003 | 0.233333333 | 4 | 44560803 | 1-{4-[7-Amino-3-(1-methyl-1H-pyrazol-4-yl)pyrazolo[1,5-a]pyrimidin-6- |
| | | | | | yl]phenyl}-3-(3-trifluoromethylphenyl)urea |
| 0.0038 | 0.016 | 0.2375 | 2 | 24823250 | 6-(6,7-dimethoxyquinolin-4-yl)oxy-N-(2,2,2-trifluoroethyl)naphthalene-1- |
| | | | | | carboxamide |
| 0.015 | 0.062 | 0.241935484 | 2 | 24823090 | 6-(6,7-dimethoxyquinolin-4-yl)oxy-N-(2,2,2-trifluoroethyl)naphthalene-1- |
| | | | | | carboxamide |
| 0.01 | 0.04 | 0.25 | 4 | 5329057 | 4-Anilinoquinazoline 31 |
| 0.5 | 2 | 0.25 | 4 | 11359538 | N-benzyl-3-phenyl-1H-thieno[2,3-c]pyrazole-5-carboxamide |
| 0.001 | 0.004 | 0.25 | 2 | 16043241 | 1-(4-(4-Aminoisothiazolo[5,4-d]pyrimidin-3-yl)phenyl)-3-m-tolylurea |
| 0.01 | 0.04 | 0.25 | 3 | 5329028 | Anilinoquinazoline deriv. 30 |
| 0.182 | 0.698 | 0.260744986 | 12 | 127027096 | 4-[4-(4-chloroanilino)-7-methoxyquinazolin-6-yl]oxy-N- |
| | | | | | hydroxybutanamide |
| 0.012 | 0.045 | 0.266666667 | 4 | 136299423 | methyl 2-hydroxy-3-[N-[4-[3-[(4-methylpiperazin-1-yl)methyl]-2- |
| | | | | | oxopyrrolidin-1-yl]phenyl]-C-phenylcarbonimidoyl]-1H-indole-6- |
| | | | | | carboxylate |
| 0.008 | 0.03 | 0.266666667 | 3 | 22478260 | (E)-4-(4-bromo2-fluoroanilino)-6-methoxy-7-(4-pyrrolidin-1-ylbut-2-en- |
| | | | | | 1-yloxy)quinazoline hydrochloride |
| 0.007 | 0.025 | 0.28 | 3 | 49860186 | 4-Fluoro-5-[6-methoxy-7-[(1-methylpiperidin-4-yl)methoxy]quinazolin- |
| | | | | | 4-yl]amino]-2-methylphenol;hydrochloride |
| 0.376 | 1.34 | 0.280597015 | 12 | 127026070 | 4-[4-(4-fluoroanilino)-7-methoxyquinazolin-6-yl]oxy-N- |
| | | | | | hydroxybutanamide |
| 0.009 | 0.032 | 0.28125 | 3 | 5329447 | 4-[2-[4-(3-Phenylpyrazolo[1,5-a]pyrimidin-6- |
| | | | | | yl)phenoxylethyl]morpholine |
| 0.09333 | 0.326 | 0.286288344 | 4 | 5330391 | Pyrazine-Pyridine Biheteroaryl 57 |
| 0.03 | 0.1 | 0.3 | 4 | 4913175 | N-(4-chloro-2-fluorophenyl)-6-methoxy-7-[2-(4-methylpiperazin-1- |
| | | | | | yl)ethoxy]quinazolin-4-amine |
| 0.015 | 0.05 | 0.3 | 4 | 23133320 | 4-(4-Chloro-2-fluoroanilino)-6-methoxy-7-(1-methylpiperidin-4- |
| | | | | | ylmethoxy)quinazoline hydrochloride |
| 0.009 | 0.03 | 0.3 | 4 | 49860190 | N-(4-bromo-2-fluorophenyl)-6-methoxy-7-[3-(4-methylpiperazin-1- |
| | | | | | yl)propoxy]quinazolin-4-amine;hydrochloride |
| 0.027 | 0.084 | 0.321428571 | 3 | 155562216 | [2-Oxo-2-[3-(pyridine-2-carbonylamino)anilino]ethyl] piperidine-1- |
| | | | | | carbodithioate |
| 0.004 | 0.012 | 0.333333333 | 5 | 25144294 | N-[6-methyl-3-(1H-pyrazol-4-yl)imidazo[1,2-a]pyrazin-8-yl]-3-(piperidin- |
| | | | | | 1-ylmethyl)-1,2-thiazol-5-amine |
| 0.0065 | 0.018 | 0.361111111 | 3 | 134155961 | [5-amino-1-(2-methyl-1H-indol-5-yl)pyrazol-4-yl]-(1H-indol-2- |
| | | | | | yl)methanone |
| 0.048 | 0.13 | 0.369230769 | 3 | 46890315 | 1-Ethyl-3-(4-(3-(2-(methylamino)pyrimidin-4-yl)pyridin-2- |
| | | | | | yloxy)naphthalen-1-yl)urea |
| 0.025 | 0.065 | 0.384615385 | 3 | 5329042 | N-(2-fluoro-4-methylphenyl)-6-methoxy-7-[(1-methylpiperidin-4- |
| | | | | | yl)methoxy]quinazolin-4-amine |
| 0.034 | 0.088 | 0.386363636 | 7 | 136002661 | 5-(2-Chlorophenyl)-1,10-dihydropyrazolo[3,4-b][1,4]benzodiazepin-7- |
| | | | | | amine |
| 0.04 | 0.1 | 0.4 | 3 | 5329050 | N-(4-chloro-2,6-difluorophenyl)-6-methoxy-7-(piperidin-4- |
| | | | | | ylmethoxy)quinazolin-4-amine |
| 0.02 | 0.05 | 0.4 | 4 | 21701597 | N-(4-chloro-2-fluorophenyl)-6-methoxy-7-[3-(4-methylpiperazin-1- |
| | | | | | yl)propoxy]quinazolin-4-amine;hydrochloride |
| 0.14 | 0.35 | 0.4 | 6 | 5908088 | (Z)-3-((1H-pyrrol-2-yl)methylene)indolin-2-one |
| 1.5 | 3.7 | 0.405405405 | 4 | 24823091 | N-tert-butyl-6-(6,7-dimethoxyquinolin-4-yl)oxynaphthalene-1- |
| | | | | | carboxamide |
| 0.00127 | 0.00308 | 0.412337662 | 6 | 9811611 | 3-((4-Bromo-2,6-difluorobenzyl)oxy)-5-(3-(4-(pyrrolidin-1- |
| | | | | | yl)butyl)ureido)isothiazole-4-carboxamide |
| 0.025 | 0.06 | 0.416666667 | 3 | 5329045 | N-(4-chloro-2,6-difluorophenyl)-6-methoxy-7-[(1-methylpiperidin-4- |
| | | | | | yl)methoxy]quinazolin-4-amine |
| 0.01 | 0.024 | 0.416666667 | 4 | 135398512 | 5-(2-Chlorophenyl)-7-fluoro-1,2-dihydro-8-methoxy-3- |
| | | | | | methylpyrazolo(3,4-b)(1,4)benzodiazepine |
| 0.012 | 0.028 | 0.428571429 | 7 | 9910609 | (+)-(1R,3R)-1-(3-hydroxy-cyclopentyl)-3-(4-methoxy-phenyl)-7- |
| | | | | | phenylamino-3,4-dihydro-1H-pyrimido[4,5-d]pyrimidin-2-one |
| 0.001 | 0.0023 | 0.434782609 | 3 | 46182735 | (Z)-1-[2-Oxo-3-(1H-pyrrol-2-ylmethylene)-2,3-dihydro-1H-indol-6-yl]-3- |
| | | | | | phenyl-urea |
| 0.067 | 0.153 | 0.437908497 | 4 | 5330392 | Pyrazine-Pyridine Biheteroaryl 58 |
| 0.015 | 0.033 | 0.454545455 | 7 | 11407794 | (-)-(1S,3S)-1-(3-hydroxy-cyclopentyl)-3-(4-methoxy-phenyl)-7- |
| | | | | | phenylamino-3,4-dihydro-1H-pyrimido[4,5-d]pyrimidin-2-one |
| 0.96 | 2.1 | 0.457142857 | 6 | 44564817 | N-(4-(benzo[d]thiazol-2-yl)-3-methoxyphenyl)-2-methoxy-9H-purin-6- |
| | | | | | amine |
| 0.00046 | 0.001 | 0.46 | 3 | 122177477 | 1-(3-tert-butyl-1,2-oxazol-5-yl)-3-[2-fluoro-5-[7-(methylamino)-2-oxo-1- |
| | | | | | [(3S)-oxolan-3-yl]-1,6-naphthyridin-3-yl]phenyl]urea |
| 0.23 | 0.5 | 0.46 | 4 | 5329040 | 4-Anilinoquinazoline 12 |
| 0.053 | 0.113 | 0.469026549 | 4 | 44440254 | 2-methoxy-N-{3-[5-(3-phenoxy-prop-1-ynyl)-thiophen-3-yl]-2,4-dihydro- |
| | | | | | indeno[1,2-c]pyrazol-5-yl}-acetamide |
| 0.025 | 0.053 | 0.471698113 | 3 | 22273644 | 5-isopropyl-6-(5-methyl-1,3,4-oxadiazol-2-yl)-N-(2-methyl-1H- |
| | | | | | pyrrolo[2,3-b]pyridin-5-yl)pyrrolo[2,1-f] [1,2,4]triazin-4-amine |
| 0.052 | 0.11 | 0.472727273 | 6 | 134146580 | [5-amino-1-(1H-indol-5-yl)pyrazol-4-yl]-(1H-indol-2-yl)methanone |
| 0.3 | 0.6 | 0.5 | 7 | 5329034 | Anilinoquinazoline deriv. 36 |
| 0.001 | 0.002 | 0.5 | 5 | 6539985 | 1-[4-(4-Aminothieno[2,3-d]pyrimidin-5-yl)phenyl]-3-(3- |
| | | | | | methylphenyl)urea |
| 0.0025 | 0.005 | 0.5 | 3 | 11530963 | |
| 0.01 | 0.02 | 0.5 | 8 | 135947979 | 6-(4-amino-1-isopropyl-1H-pyrazolo[3,4-d]pyrimidin-3-yl)naphthalen-2- |
| | | | | | ol |
| 0.004 | 0.008 | 0.5 | 4 | 44447890 | 7-[(1E)-1-(methoxyimino)ethyl]-19-methyl-3-(2-methylpropyl)- |
| | | | | | 3,13,19,20- |
| | | | | | tetraazahexacyclo[14.7.0.0\hat{ }{2,10}.0\hat{ }{4,9}.0\hat{ }{11,15}.0\hat{ }{17,21}] |
| | | | | | tricosa-1(16),2(10),4,6,8,11(15), 17,20-octaen-14-one |
| 0.005012 | 0.01 | 0.5012 | 4 | 10113978 | Pazopanib |
| 0.031 | 0.061 | 0.508196721 | 4 | 44449577 | 6-(5-methyl-1,3,4-oxadiazol-2-yl)-5-propan-2-yl-N-(1H-pyrrolo[2,3- |
| | | | | | b]pyridin-5-yl)pyrrolo[2,1-f\|1,2,4]triazin-4-amine |
| 0.001 | 0.0018 | 0.555555556 | 5 | 135423438 | Nintedanib |
| 1.13 | 2 | 0.565 | 4 | 5703472 | 3-[2,2″]Bithiophenyl-5-ylmethylene-1,3-dihydro-indol-2-one |
| 0.194 | 0.341 | 0.568914956 | 4 | 24905198 | 2-(4-(4-amino-1-isopropyl-1H-pyrazolo[3,4-d]pyrimidin-3-yl)-2- |
| | | | | | methoxyphenylamino)propan-1-ol |
| 0.0055 | 0.0096 | 0.572916667 | 2 | 56835147 | Urea, N-(3-fluorophenyl)-N′-[4-[7-[(1-methyl-4-piperidinyl)methoxy]-4- |
| | | | | | quinazolinyl]amino]phenyl]- |
| 0.003 | 0.005 | 0.6 | 3 | 44442418 | 1-(5-((5-((2,2-Difluorobenzo[d][1,3]dioxol-5-yl)carbamoyl)thiazol-4- |
| | | | | | ylamino)methyl)pyridin-2-yl)-3-methylurea |
| 0.003 | 0.005 | 0.6 | 4 | 67161862 | 5-[(1-ethylpiperidin-4-yl)amino]-3-[(2-fluorophenyl)-(5-methyl-1H- |
| | | | | | imidazol-2-yl)methylidene]-1H-indol-2-one |
| 0.003 | 0.005 | 0.6 | 3 | 135488014 | 3-[benzimidazol-2-ylidene-(3-fluorophenyl)methyl]-5-[(1-ethylpiperidin- |
| | | | | | 4-yl)amino]-1H-indol-2-ol |
| 0.018 | 0.03 | 0.6 | 5 | 11429729 | 3-[4-(3-Amino-7-methoxy-1,2-benzoxazol-4-yl)phenyl]-1-(3- |
| | | | | | chlorophenyl)urea |
| 0.03 | 0.05 | 0.6 | 4 | 5329032 | N-(4-bromo-2-fluorophenyl)-6-methoxy-7-[2-(triazol-1- |
| | | | | | yl)ethoxy]quinazolin-4-amine |
| 0.05 | 0.083 | 0.602409639 | 8 | 11396738 | 7-(4-fluoroanilino)-1-[(1R,3R)-3-hydroxycyclopentyl]-3-(4- |
| | | | | | methoxyphenyl)-4H-pyrimido[4,5-d]pyrimidin-2-one |
| 0.00235 | 0.00387 | 0.607235142 | 4 | 145949428 | N-[5-[4-[(3-chlorophenyl)carbamoylamino]phenyl]pyridin-2-yl]prop-2- |
| | | | | | enamide |
| 0.33 | 0.54 | 0.611111111 | 3 | 155536201 | 1,5-bis[(1S)-5-hydroxy-1-methyl-1,2-dihydrobenzo[e]indol-3-yl]pentane- |
| | | | | | 1,5-dione |
| 0.012 | 0.019 | 0.631578947 | 5 | 71466525 | 1-N′-[3-fluoro-4-[7-[4-(hydroxyamino)-4-oxobutoxy]-6-methoxyquinolin- |
| | | | | | 4-yl]oxyphenyl]-1-N-(4-fluorophenyl)cyclopropane-1,1-dicarboxamide |
| 0.011 | 0.017 | 0.647058824 | 2 | 122193773 | 1-(3-Bromophenyl)-3-[4-(5,6,7,8-tetrahydro-[1]benzothiolo[2,3- |
| | | | | | d]pyrimidin-4-yloxy)phenyl]urea |
| 0.0065 | 0.0099 | 0.656565657 | 6 | 141475848 | methyl 2-hydroxy-3-[N-[4-[2-methyl-5-[(4-methylpiperazin-1- |
| | | | | | yl)methyl]pyrrol-1-yl]phenyl]-C-phenylcarbonimidoyl]-1H-indole-6- |
| | | | | | carboxylate |
| 0.0033 | 0.005 | 0.66 | 7 | 46179972 | |
| 0.004 | 0.006 | 0.666666667 | 3 | 135546228 | 3-[benzimidazol-2-ylidene-(3,5-difluorophenyl)methyl]-5-[(1- |
| | | | | | ethylpiperidin-4-yl)amino]-1H-indol-2-ol |
| 0.04 | 0.06 | 0.666666667 | 4 | 23133323 | Vandetanib hydrochloride |
| 0.02 | 0.03 | 0.666666667 | 2 | 5289418 | 3-[(3-(2-Carboxyethyl)-4-methylpyrrol-2-YL)methylene]-2-indolinone |
| | | | | | (SU5402) |
| 0.857 | 1.255 | 0.682868526 | 12 | 127027019 | 4-[4-(2-chloroanilino)-7-methoxyquinazolin-6-yl]oxy-N- |
| | | | | | hydroxybutanamide |
| 1.3 | 1.9 | 0.684210526 | 12 | 2132479 | 1-Methyl-3,5-bis-pyridin-2-ylmethylene-piperidin-4-one |
| 0.011 | 0.016 | 0.6875 | 5 | 11374110 | 3-[4-(3-Amino-7-methoxy-1,2-benzoxazol-4-yl)phenyl]-1-[3- |
| | | | | | (trifluoromethyl)phenyl]urea |
| 3.1 | 4.5 | 0.688888889 | 6 | 24894886 | 3-(3,4,5-Trimethoxyphenyl)-4-(1-{[2-(trimethylsilyl)ethoxy]-methyl}-1H- |
| | | | | | indol-3-yl)-1,5-dihydro-2H-pyrrole-2-one |
| 0.07 | 0.1 | 0.7 | 3 | 5329169 | Indolin-2-one deriv. 9g |
| 2.3 | 3.2 | 0.71875 | 2 | 24894887 | 4-[1-(Methoxymethyl)-1H-indol-3-yl]-3-(3,4,5-trimethoxyphenyl)-1,5- |
| | | | | | dihydro-2H-pyrrole-2-one |
| 0.016 | 0.022 | 0.727272727 | 5 | 44414779 | 1-(4-(4-Aminoisothiazolo[5,4-d]pyrimidin-3-yl)phenyl)-3-(2-fluoro-5- |
| | | | | | (trifluoromethyl)phenyl)urea |
| 0.00238 | 0.00326 | 0.73006135 | 4 | 118737468 | 1-[4-[6, 7-Bis(trideuteriomethoxy)quinolin-4-yl]oxy-2-chlorophenyl]-3-(5- |
| | | | | | methyl-1,2-oxazol-3-yl)urea |
| 0.476 | 0.645 | 0.737984496 | 12 | 122195525 | 3-[4-(3-chloroanilino)-7-methoxyquinazolin-6-yl]oxy-N- |
| | | | | | hydroxypropanamide |
| 12 | 16 | 0.75 | 5 | 44405941 | N-benzyl-3-methyl-1H-thieno[2,3-c]pyrazole-5-carboxamide |
| 0.0093 | 0.0123 | 0.756097561 | 18 | 44555430 | N-(4-methoxyphenyl)-N,5-dimethylfuro[2,3-d]pyrimidin-4-amine |
| 0.00016 | 0.00021 | 0.761904762 | 3 | 9911830 | Tivozanib |
| 0.0023 | 0.003 | 0.766666667 | 8 | 11485656 | Linifanib |
| 1.56 | 2.02 | 0.772277228 | 7 | 57332840 | N-(4-Piperidinophenyl)-2-phenylpyrido[3,4-b]pyrazine-8-amine |
| 0.0016 | 0.00204 | 0.784313725 | 5 | 68620328 | 1-[4-[4-amino-7-[1-[(2S)-2-hydroxypropyl]pyrazol-4-yl]thieno[3,2- |
| | | | | | c]pyridin-3-yl]phenyl]-3-(3-methylphenyl)urea |
| 0.055 | 0.07 | 0.785714286 | 4 | 5329052 | N-(4-chloro-2-fluorophenyl)-6-methoxy-7-[2-(1-methylpiperidin-4- |
| | | | | | yl)ethoxy]quinazolin-4-amine |
| 0.00016 | 0.0002 | 0.8 | 6 | 216239 | Sorafenib |
| 0.0058 | 0.00723 | 0.802213001 | 2 | 53324451 | (4S,6Z,10S)-10,18-dihydroxy-16-methoxy-4-methyl-3- |
| | | | | | oxabicyclo[12.4.0]octadeca-1(14),6,15,17-tetraene-2,8-dione |
| 10.3 | 12.8 | 0.8046875 | 6 | 6477203 | 5-[(1e)-2-(2-Methoxyphenyl)prop-1-En-1-Yl]furo[2,3-D]pyrimidine-2,4- |
| | | | | | Diamine |
| 0.00509 | 0.00627 | 0.811802233 | 2 | 68245 | Delphinidin |
| 0.013 | 0.016 | 0.8125 | 9 | 57339386 | 6-(4-methoxyphenyl)-3-(1H-pyrazol-4-yl)pyrazolo[1,5-a]pyrimidine |
| 0.314 | 0.385 | 0.815584416 | 2 | 24823252 | 6-(6,7-dimethoxyquinazolin-4-yl)oxy-N-(2-methoxyethyl)naphthalene-1- |
| | | | | | carboxamide |
| 0.009 | 0.011 | 0.818181818 | 7 | 3081361 | Vandetanib |
| 0.05 | 0.06 | 0.833333333 | 4 | 73349643 | N-(4-chloro-2-fluorophenyl)-6-methoxy-7-(piperidin-4- |
| | | | | | ylmethoxy)quinazolin-4-amine;hydrochloride |
| 0.02 | 0.024 | 0.833333333 | 6 | 11429654 | 3-[4-(3-Amino-7-methoxy-1,2-benzoxazol-4-yl)phenyl]-1-(2-fluoro-5- |
| | | | | | methylphenyl)urea |
| 0.0191 | 0.0228 | 0.837719298 | 4 | 44611919 | |
| 0.031 | 0.037 | 0.837837838 | 2 | 24894117 | 4-(1H-Indol-3-yl)-3-(3,4,5-trimethoxyphenyl)-1,5-dihydro-2H-pyrrole-2- |
| | | | | | one |
| 0.02489 | 0.02951 | 0.843442901 | 3 | 46231220 | 3-(5-Iodo-1H-indol-3-yl)-4-(3,4,5-trimethoxyphenyl)-1H-pyrrole-2,5- |
| | | | | | dione |
| 0.0071 | 0.0084 | 0.845238095 | 4 | 57505528 | N-[3-(imidazo[1,2-b]pyridazin-6-yloxy)phenyl]-3- |
| | | | | | (trifluoromethyl)benzamide |
| 0.0017 | 0.002 | 0.85 | 5 | 11495270 | Urea, N-[4-(3-amino-1H-pyrazolo[3,4-b]pyridin-4-yl)phenyl]-N′-[3- |
| | | | | | (trifluoromethyl)phenyl]- |
| 0.012 | 0.014 | 0.857142857 | 7 | 46225612 | N-(4-(benzo[d]thiazol-2-yl)phenyl)-6-methoxy-7-(3-(4-methylpiperazin-1- |
| | | | | | yl)propoxy)quinazolin-4-amine |
| 0.006 | 0.007 | 0.857142857 | 4 | 135399795 | methyl 2-hydroxy-3-[N-[4-[(2-imidazol-1-ylacetyl)-methylamino]phenyl]- |
| | | | | | C-phenylcarbonimidoyl]-1H-indole-6-carboxylate |
| 1.3 | 1.5 | 0.866666667 | 24 | 10396070 | Bastadin 7 |
| 0.059 | 0.068 | 0.867647059 | 12 | 127028028 | 5-[4-(4-bromoanilino)-7-methoxyquinazolin-6-yl]oxy-N- |
| | | | | | hydroxypentanamide |
| 0.033 | 0.038 | 0.868421053 | 7 | 136002662 | N-[5-(2-chlorophenyl)-1,10-dihydropyrazolo[3,4-b][1,4]benzodiazepin-7- |
| | | | | | yl]acetamide |
| 2.06 | 2.34 | 0.88034188 | 7 | 127030424 | 3-phenyl-N-(4-piperidin-1-ylphenyl)pyrido[3,4-b]pyrazin-8-amine |
| 1.5 | 1.7 | 0.882352941 | 5 | 24894997 | 4-[1-(Phenoxymethyl)-1H-indol-3-yl]-3-(3,4,5-trimethoxyphenyl)-1,5- |
| | | | | | dihydro-2H-pyrrole-2-one |
| 8 | 9 | 0.888888889 | 5 | 44405999 | 4-(2-chlorobenzamido)-3-methyl-1H-thieno[2,3-c]pyrazole-5-carboxamide |
| 0.245 | 0.275 | 0.890909091 | 5 | 46879446 | 3-(1-(6-(2-cyanoethylamino)pyrimidin-4-yl)-1H-benzo[d]imidazol-2- |
| | | | | | ylamino)-4-methyl-N-(3-(trifluoromethyl)phenyl)benzamide |
| 0.025 | 0.028 | 0.892857143 | 3 | 11234052 | Brivanib |
| 0.0018 | 0.002 | 0.9 | 5 | 11588815 | N-[4-(3-amino-1-methyl-1H-pyrazolo[3,4-c]pyridin-4-yl)phenyl]-N′-(3- |
| | | | | | methylphenyl)urea |
| 2.05 | 2.27 | 0.9030837 | 7 | 57332844 | 2-(4-methoxyphenyl)-N-(4-piperidin-1-ylphenyl)pyrido[3,4-b]pyrazin-8- |
| | | | | | amine |
| 1.2 | 1.3 | 0.923076923 | 9 | 44564789 | N-(4-(benzo[d]thiazol-2-yl)phenyl)-7H-pyrrolo[2,3-d]pyrimidin-4-amine |
| 0.029 | 0.031 | 0.935483871 | 8 | 56924694 | 4-(4-Fluorophenyl)-3-(pyridin-4-yl)-1-(2,4,6-trichlorophenyl)-1H-pyrazol- |
| | | | | | 5-amine |
| 1.6 | 1.7 | 0.941176471 | 5 | 24894993 | 4-{1-[(Benzyloxy)methyl]-1H-indol-3-yl}-3-(3,4,5-trimethoxyphenyl)- |
| | | | | | 1,5-dihydro-2H-pyrrole-2-one |
| 0.033 | 0.035 | 0.942857143 | 5 | 11755812 | 1-(3-(8-(Pyridin-4-ylmethylamino)imidazo[1,2-a]pyrazin-6-yl)phenyl)-3- |
| | | | | | (3-(trifluoromethyl)phenyl)urea |
| 0.048 | 0.05 | 0.96 | 6 | 15949619 | N-(3-(1-(6-aminopyrimidin-4-yl)-1H-benzo[d]imidazol-2-ylamino)-4- |
| | | | | | methylphenyl)-3-cyclopropyl-1H-pyrazole-5-carboxamide |
| 5.2 | 5.3 | 0.981132075 | 7 | 24894996 | 4-[1-(Isopropoxymethyl)-1H-indol-3-yl]-3-(3,4,5-trimethoxyphenyl)-1,5- |
| | | | | | dihydro-2H-pyrrole-2-one |
| 0.05 | 0.05 | 1 | 4 | 57802779 | N-(2,3-dichloro-4-methylphenyl)-6-methoxy-7-[(4-methylmorpholin-2- |
| | | | | | yl)methoxy quinazolin-4-amine |
| 0.05 | 0.05 | 1 | 5 | 57802653 | N-(3,4-dichloro-2-fluorophenyl)-6-methoxy-7-[(4,5,5-trimethylmorpholin- |
| | | | | | 2-yl)methoxy]quinazolin-4-amine |
| 0.05 | 0.05 | 1 | 5 | 57802713 | N-(3,4-dichlorophenyl)-6-ethoxy-7-(morpholin-2-ylmethoxy)quinazolin-4- |
| | | | | | amine |
| 0.05 | 0.05 | 1 | 5 | 57802695 | N-(3,4-dichlorophenyl)-6-ethoxy-7-[(4-methylmorpholin-2- |
| | | | | | yl)methoxy]quinazolin-4-amine |
| 0.1 | 0.1 | 1 | 13 | 25071593 | 4-[4-[[4-chloro-3-(trifluoromethyl)phenyl]carbamoylamino]phenoxy]-N- |
| | | | | | [8-(hydroxyamino)-8-oxooctyl]pyridine-2-carboxamide |
| 0.05 | 0.05 | 1 | 3 | 57802786 | N-(4-bromo-3-chlorophenyl)-6-methoxy-7-(1-methylpyrrolidin-3- |
| | | | | | yl)oxyquinazolin-4-amine |
| 0.05 | 0.05 | 1 | 4 | 57802721 | (6S)-6-[[4-(4-bromo-3-chloro-2-fluoroanilino)-6-methoxyquinazolin-7- |
| | | | | | yl]oxymethyl]-1,4-dimethylpiperazin-2-one |
| 0.05 | 0.05 | 1 | 5 | 57802797 | N-(3,4-dichlorophenyl)-7-[(4-methylmorpholin-2-yl)methoxy]-6-propan- |
| | | | | | 2-yloxyquinazolin-4-amine |
| 0.05 | 0.05 | 1 | 5 | 57802800 | U.S. Pat. No. 9,796,704, Entry 194 |
| 0.05 | 0.05 | 1 | 5 | 57802801 | 6-methoxy-7-[(4-methylmorpholin-2-yl)methoxy]-N-(2,3,4- |
| | | | | | trichlorophenyl)quinazolin-4-amine |
| 0.05 | 0.05 | 1 | 5 | 57802650 | N-(3-chloro-2,4-difluorophenyl)-6-methoxy-7-[(4,5,5-trimethylmorpholin- |
| | | | | | 2-yl)methoxy\|quinazolin-4-amine |
| 0.05 | 0.05 | 1 | 5 | 57802643 | U.S. Pat. No. 9,796,704, Entry 182 |
| 0.05 | 0.05 | 1 | 5 | 57802656 | 7-[[4-(3-chloropropylsulfonyl)morpholin-2-yl]methoxy]-N-(3,4- |
| | | | | | dichlorophenyl)-6-methoxyquinazolin-4-amine |
| 0.004 | 0.004 | 1 | 5 | 15949617 | N-(3-(1-(6-aminopyrimidin-4-yl)-1H-benzo[d]imidazol-2-ylamino)-4- |
| | | | | | methylphenyl)-5-tert-butylthiophene-2-carboxamide |
| 0.05 | 0.05 | 1 | 6 | 57802698 | U.S. Pat. No. 9,796,704, Entry 56 |
| 0.05 | 0.05 | 1 | 5 | 57802776 | N-(4-bromo-5-chloro-2-fluorophenyl)-6-methoxy-7-[(4,5,5- |
| | | | | | trimethylmorpholin-2-yl)methoxy]quinazolin-4-amine |
| 0.05 | 0.05 | 1 | 5 | 57802694 | (2-Chloropyridin-3-yl)-[2-[[4-(3,4-dichloroanilino)-6-methoxyquinazolin- |
| | | | | | 7-yl]oxymethyl]morpholin-4-yl]methanone |
| 0.05 | 0.05 | 1 | 5 | 57802736 | [2-[4-(3,4-Dichloroanilino)-6-methoxyquinazolin-7- |
| | | | | | yl]oxymethyl]morpholin-4-yl]-piperidin-1-ylmethylidene]cyanamide |
| 0.1 | 0.1 | 1 | 12 | 25072218 | 8-[[4-[4-[[4-chloro-3- |
| | | | | | (trifluoromethyl)phenyl]carbamoylamino]phenoxy]pyridin-2- |
| | | | | | yl]methylamino]-N-hydroxyoctanamide |
| 0.05 | 0.05 | 1 | 5 | 57802680 | N-(3-chloro-2,4-difluorophenyl)-6-methoxy-7-[(4-methylmorpholin-2- |
| | | | | | yl)methoxy]quinazolin-4-amine |
| 0.05 | 0.05 | 1 | 5 | 57802679 | U.S. Pat. No. 9,796,704, Entry 216 |
| 0.05 | 0.05 | 1 | 6 | 57802678 | U.S. Pat. No. 9,796,704, Entry 34 |
| 0.05 | 0.05 | 1 | 5 | 57802725 | 1-[2-[[4-(3,4-Dichloroanilino)-6-methoxyquinazolin-7-yl]oxymethyl]-1,4- |
| | | | | | oxazepan-4-yllethanone |
| 0.05 | 0.05 | 1 | 5 | 57802675 | U.S. Pat. No. 9,796,704, Entry 258 |
| 0.05 | 0.05 | 1 | 5 | 57802669 | N-(4-bromo-2,3-dichlorophenyl)-6-methoxy-7-[(4-methylmorpholin-2- |
| | | | | | yl)methoxy]quinazolin-4-amine |
| 0.05 | 0.05 | 1 | 5 | 57802747 | N-(4-bromo-3-chloro-2-fluorophenyl)-6-methoxy-7-[(4,5,5- |
| | | | | | trimethylmorpholin-2-yl)methoxy]quinazolin-4-amine |
| 0.1 | 0.1 | 1 | 12 | 25072217 | 7-[[4-[4-[[4-chloro-3- |
| | | | | | (trifluoromethyl)phenyl]carbamoylamino]phenoxy]pyridin-2- |
| | | | | | yl]methylamino]-N-hydroxyheptanamide |
| 0.05 | 0.05 | 1 | 5 | 57802660 | N-(3,4-dichlorophenyl)-6-methoxy-7-[(4,5,5-trimethylmorpholin-2- |
| | | | | | yl)methoxy\|quinazolin-4-amine |
| 0.05 | 0.05 | 1 | 6 | 57802722 | U.S. Pat. No. 9,796,704, Entry 39 |
| 0.05 | 0.05 | 1 | 5 | 57802754 | U.S. Pat. No. 9,796,704, Entry 193 |
| 0.05 | 0.05 | 1 | 5 | 57802766 | N-(4-bromo-3-chlorophenyl)-6-methoxy-7-[(4-methylmorpholin-2- |
| | | | | | yl)methoxy]quinazolin-4-amine |
| 0.05 | 0.05 | 1 | 4 | 57802732 | N-(3,4-dichlorophenyl)-6-methoxy-7-[2-(4,6,6-trimethylmorpholin-2- |
| | | | | | yl)ethoxy]quinazolin-4-amine |
| 0.05 | 0.05 | 1 | 5 | 57802737 | U.S. Pat. No. 9,796,704, Entry 212 |
| 0.05 | 0.05 | 1 | 5 | 57802578 | N-(4-bromo-2,3-dichlorophenyl)-6-methoxy-7-[(4,5,5- |
| | | | | | trimethylmorpholin-2-yl)methoxy\|quinazolin-4-amine |
| 0.05 | 0.05 | 1 | 5 | 57802637 | N-(3,4-dichlorophenyl)-7-[2-(5,5-dimethylmorpholin-2-yl)ethoxy]-6- |
| | | | | | methoxyquinazolin-4-amine |
| 0.1 | 0.1 | 1 | 13 | 24900442 | N-hydroxy-6-[4-[[4-[4-[[(1R)-1-phenylethyl]amino]-7H-pyrrolo[2,3- |
| | | | | | d]pyrimidin-6-yl]phenyl]methyl]piperazin-1-yl]hexanamide |
| 0.05 | 0.05 | 1 | 5 | 57802807 | U.S. Pat. No. 9,796,704, Entry 53 |
| 0.1 | 0.1 | 1 | 12 | 24900443 | N-hydroxy-7-[4-[[4-[4-[[(1R)-1-phenylethyl]amino]-7H-pyrrolo[2,3- |
| | | | | | d]pyrimidin-6-yl]phenyl]methyl]piperazin-1-yl]heptanamide |
| 0.1 | 0.1 | 1 | 13 | 24900506 | N-hydroxy-4-[4-[4-[[(1R)-1-phenylethyl]amino]-7H-pyrrolo[2,3- |
| | | | | | d]pyrimidin-6-yl]phenoxy]butanamide |
| 0.1 | 0.1 | 1 | 13 | 24900507 | N-hydroxy-5-[4-[4-[[(1R)-1-phenylethyl]amino]-7H-pyrrolo[2,3- |
| | | | | | d]pyrimidin-6-yl]phenoxy]pentanamide |
| 0.1 | 0.1 | 1 | 13 | 24900574 | N′-hydroxy-N-[4-[4-[[(1R)-1-phenylethyl]amino]-7H-pyrrolo[2,3- |
| | | | | | d]pyrimidin-6-yl]phenyl]pentanediamide |
| 0.1 | 0.1 | 1 | 13 | 24900641 | N-[2-[[6-(hydroxyamino)-6-oxohexyl]amino]ethyl]-4-[4-[[(1R)-1- |
| | | | | | phenylethyl]amino]-7H-pyrrolo[2,3-d]pyrimidin-6-yl]benzamide |
| 0.1 | 0.1 | 1 | 13 | 24900643 | N-hydroxy-6-[[4-[4-[[(1R)-1-phenylethyl]amino]-7H-pyrrolo[2,3- |
| | | | | | d]pyrimidin-6-yl]phenyl]methylamino]hexanamide |
| 0.1 | 0.1 | 1 | 14 | 24900644 | N-hydroxy-8-[[4-[4-[[(1R)-1-phenylethyl]amino]-7H-pyrrolo[2,3- |
| | | | | | d]pyrimidin-6-yl]phenyl]methylamino]octanamide |
| 0.1 | 0.1 | 1 | 13 | 24900441 | N-hydroxy-5-[4-[[4-[4-[[(1R)-1-phenylethyl]amino]-7H-pyrrolo[2,3- |
| | | | | | d]pyrimidin-6-yl]phenyl]methyl]piperazin-1-yl]pentanamide |
| 0.0005 | 0.0005 | 1 | 7 | 46898039 | (Z)-5-{6-[3-(4-Methoxyphenyl)-ureido]-2-oxo-1,2-dihydroindol-3- |
| | | | | | ylidene-methyl}-4-methyl-1H-pyrrole-3-carboxylic acid |
| 0.1 | 0.1 | 1 | 12 | 24900715 | N-hydroxy-4-[4-[[(1R)-1-phenylethyl]amino]-7H-pyrrolo[2,3- |
| | | | | | d]pyrimidin-6-yl]benzamide |
| 0.1 | 0.1 | 1 | 13 | 24900772 | 4-[4-[[(1R)-1-(4-fluorophenyl)ethyl]amino]-7H-pyrrolo[2,3-d]pyrimidin- |
| | | | | | 6-yl]-N-hydroxybenzamide |
| 0.05 | 0.05 | 1 | 5 | 53630972 | N-(3,4-dichlorophenyl)-6-(methyloxy)-7-{[(2-piperidin-4-yl-1,3-thiazol-4- |
| | | | | | yl)methyl]oxy}quinazolin-4-amine |
| 0.001 | 0.001 | 1 | 3 | 52944268 | 4-(6-(4-(2-Morpholinoethoxy)phenyl)pyrazolo[1,5-a]pyrimidin-3- |
| | | | | | yl)benzene-1,2-diol |
| 0.05 | 0.05 | 1 | 5 | 53630967 | N-(3,4-dichlorophenyl)-6-(methyloxy)-7-[(4,5,6,7- |
| | | | | | tetrahydro[1,3]thiazolo[5,4-c]pyridin-2-ylmethyl)oxy]quinazolin-4-amine |
| 0.05 | 0.05 | 1 | 5 | 53630924 | (3S,8aR)-3-({[4-[(3,4-dichloro-2-fluorophenyl)amino]-6- |
| | | | | | (methyloxy)quinazolin-7-yl]oxy}methyl)hexahydropyrrolo[1,2-a]pyrazin- |
| | | | | | 1(2H)-one |
| 0.05 | 0.05 | 1 | 6 | 53630923 | (3S,8aS)-3-({[4-[(3,4-dichloro-2-fluorophenyl)amino]-6- |
| | | | | | (methyloxy)quinazolin-7-yl]oxy}methyl)hexahydropyrrolo[1,2-a]pyrazin- |
| | | | | | 1(2H)-one |
| 0.05 | 0.05 | 1 | 5 | 53630920 | (3S,8aS)-3-[[4-(3,4-dichloro-2-fluoroanilino)-6-methoxyquinazolin-7- |
| | | | | | yl]oxymethyl]-2-methyl-3,4,6,7,8,8a-hexahydropyrrolo[1,2-a]pyrazin-1- |
| | | | | | one |
| 0.001 | 0.001 | 1 | 4 | 53317913 | SCH-1473759 hydrochloride |
| 0.1 | 0.1 | 1 | 14 | 24900714 | 6-[4-[[4-[4-(benzylamino)-7H-pyrrolo[2,3-d]pyrimidin-6- |
| | | | | | yl]phenyl methyl]piperazin-1-yl]-N-hydroxyhexanamide |
| 0.05 | 0.05 | 1 | 5 | 57802639 | (6S)-6-[[4-(3,4-dichloro-2-fluoroanilino)-6-methoxyquinazolin-7- |
| | | | | | yl]oxymethyl]-4-methylpiperazin-2-one |
| 4.4 | 4.4 | 1 | 21 | 24894992 | 4-[1-(tert-Butoxymethyl)-1H-indol-3-yl]-3-(3,4,5-trimethoxyphenyl)-1,5- |
| | | | | | dihydro-2H-pyrrole-2-one |
| 0.05 | 0.05 | 1 | 5 | 57802550 | N-(3-bromo-4-methylphenyl)-6-methoxy-7-[(4-methylmorpholin-2- |
| | | | | | yl)methoxy quinazolin-4-amine |
| 0.5 | 0.5 | 1 | 11 | 42628503 | Alw-II-41-27 |
| 0.05 | 0.05 | 1 | 5 | 57802634 | U.S. Pat. No. 9,796,704, Entry 50 |
| 0.05 | 0.05 | 1 | 5 | 57802623 | U.S. Pat. No. 9,796,704, Entry 241 |
| 0.05 | 0.05 | 1 | 5 | 57802612 | U.S. Pat. No. 9,796,704, Entry 49 |
| 0.05 | 0.05 | 1 | 5 | 57802611 | U.S. Pat. No. 9,796,704, Entry 277 |
| 0.05 | 0.05 | 1 | 5 | 57802607 | N-(3,4-dichlorophenyl)-6-methoxy-7-[[2-(1-methylpiperidin-3-yl)-1,3- |
| | | | | | thiazol-4-yl]methoxy]quinazolin-4-amine |
| 0.04 | 0.04 | 1 | 4 | 22478297 | (E)-4-(4-chloro-2-fluoroanilino)-6-methoxy-7-(4-(pyrrolidin-1-yl)but-2- |
| | | | | | en-1-yloxy)quinazoline hydrochloride |
| 0.05 | 0.05 | 1 | 5 | 57802605 | U.S. Pat. No. 9,796,704, Entry 218 |
| 0.05 | 0.05 | 1 | 5 | 57802603 | 2-[2-[[4-(3,4-Dichloroanilino)-6-methoxyquinazolin-7- |
| | | | | | yl]oxymethyl]morpholin-4-yl]acetamide |
| 0.05 | 0.05 | 1 | 5 | 57802547 | N-(4,5-dichloro-2-fluorophenyl)-6-methoxy-7-[(4-methylmorpholin-2- |
| | | | | | yl)methoxy]quinazolin-4-amine |
| 0.05 | 0.05 | 1 | 5 | 57802593 | 6-methoxy-7-[(4-methylmorpholin-2-yl)methoxy]-N-(3,4,5- |
| | | | | | trichlorophenyl)quinazolin-4-amine |
| 0.05 | 0.05 | 1 | 4 | 57802584 | N-(3,4-dichloro-2-methylphenyl)-6-methoxy-7-[(4-methylmorpholin-2- |
| | | | | | yl)methoxy]quinazolin-4-amine |
| 0.05 | 0.05 | 1 | 6 | 57802582 | U.S. Pat. No. 9,796,704, Entry 33 |
| 0.05 | 0.05 | 1 | 6 | 53630919 | N-(4,5-dichloro-2-fluorophenyl)-7-{[(3S,8aS)-hexahydro-1H-pyrrolo[2,1- |
| | | | | | c][1,4]oxazin-3-ylmethyl]oxy}-6-(methyloxy)quinazolin-4-amine |
| 0.05 | 0.05 | 1 | 5 | 57802576 | N-(3,4-dichlorophenyl)-7-(morpholin-2-ylmethoxy)-6-propan-2- |
| | | | | | yloxyquinazolin-4-amine |
| 0.05 | 0.05 | 1 | 4 | 57802574 | (6S)-6-[[4-(3,4-dichloro-2-fluoroanilino)-6-methoxyquinazolin-7- |
| | | | | | yl]oxymethyl]-1,4-dimethylpiperazin-2-one |
| 0.05 | 0.05 | 1 | 5 | 57802566 | N-(4-bromo-3-chloro-2-fluorophenyl)-6-methoxy-7-[(4-methylmorpholin- |
| | | | | | 2-yl)methoxy]quinazolin-4-amine |
| 0.05 | 0.05 | 1 | 5 | 57802565 | N-cyano-2-[[4-(3,4-dichloroanilino)-6-methoxyquinazolin-7- |
| | | | | | yl]oxymethyl]-N′-ethylmorpholine-4-carboximidamide |
| 0.05 | 0.05 | 1 | 5 | 57802560 | N-(4-bromo-2,3-difluorophenyl)-6-methoxy-7-[(4-methylmorpholin-2- |
| | | | | | yl)methoxy]quinazolin-4-amine |
| 0.05 | 0.05 | 1 | 5 | 57802556 | N-(3,4-dichlorophenyl)-6-methoxy-7-[(4-methylsulfonyl-1,4-oxazepan-2- |
| | | | | | yl)methoxy]quinazolin-4-amine |
| 0.05 | 0.05 | 1 | 5 | 57802591 | N-(3,4-dichloro-2-fluorophenyl)-6-methoxy-7-[(4-methylmorpholin-2- |
| | | | | | yl)methoxy]quinazolin-4-amine |
| 0.05 | 0.05 | 1 | 5 | 57802817 | N-(3,4-dichlorophenyl)-7-[(4-ethylsulfonylmorpholin-2-yl)methoxy]-6- |
| | | | | | methoxyquinazolin-4-amine |
| 0.05 | 0.05 | 1 | 6 | 57802793 | U.S. Pat. No. 9,796,704, Entry 36 |
| 0.6 | 0.6 | 1 | 21 | 71524406 | N-(3-Bromo-4-hydroxyphenethyl)-N′-(3-bromo-4-(3-(2-hydroxyimino-3- |
| | | | | | oxo-3-(3-bromo-4-hydroxyphenethylamino)propyl)-5-bromo-6- |
| | | | | | hydroxyphenyl)oxyphenethyl)-5,5′-dibromo-6,6′-dihydroxybiphenyl 3,3′- |
| | | | | | di(2-hydroxyiminopropionamide) |
| 0.05 | 0.05 | 1 | 5 | 66954904 | U.S. Pat. No. 9,796,704, Entry 48 |
| 0.05 | 0.05 | 1 | 6 | 10370400 | C23H23N404FClBr |
| 0.0011 | 0.0011 | 1 | 7 | 10388612 | 2,4-Difluoro-5-[5-isopropyl-6-(5-methyl-oxazol-2-yl)-pyrrolo[2,1- |
| | | | | | f][1,2,4]triazin-4-ylamino]-N-methoxy-benzamide |
| 0.05 | 0.05 | 1 | 3 | 66954887 | U.S. Pat. No. 9,796,704, Entry 46 |
| 0.05 | 0.05 | 1 | 6 | 66839071 | 7-[[(3S,8aS)-3,4,6,7,8,8a-hexahydro-1H-pyrrolo[2,1-c][1,4]oxazin-3- |
| | | | | | yl]methoxy]-N-(4-bromo-5-chloro-2-fluorophenyl)-6-methoxyquinazolin- |
| | | | | | 4-amine |
| 0.05 | 0.05 | 1 | 5 | 66838579 | N-(3,4-dichlorophenyl)-6-methoxy-7-[(5-methyl-6,7-dihydro-4H- |
| | | | | | [1,3]thiazolo[5,4-c]pyridin-2-yl)methoxy]quinazolin-4-amine |
| 0.05 | 0.05 | 1 | 6 | 66838567 | 7-[[(3S,9aS)-3,4,6,7,9,9a-hexahydro-1H-[1,4]oxazino[3,4-c][1,4]oxazin-3- |
| | | | | | yl]methoxy]-N-(3-chloro-2,4-difluorophenyl)-6-methoxyquinazolin-4- |
| | | | | | amine |
| 0.0021 | 0.0021 | 1 | 7 | 10436729 | 2,4-difluoro-N-methoxy-5-[[6-[5-(methylsulfonylmethyl)-1,3,4-oxadiazol- |
| | | | | | 2-yl]-5-propan-2-ylpyrrolo[2,1-f][1,2,4]triazin-4-y]]amino]benzamide |
| 0.05 | 0.05 | 1 | 5 | 89125605 | 2-[[4-(4-chloro-3-methylanilino)-6-methoxyquinazolin-7-yl]oxymethyl]- |
| | | | | | N-cyano-N′-methylmorpholine-4-carboximidamide |
| 0.05 | 0.05 | 1 | 6 | 66838546 | U.S. Pat. No. 9,796,704, Entry 35 |
| 0.05 | 0.05 | 1 | 5 | 10455540 | U.S. Pat. No. 9,796,704, Entry 170 |
| 0.05 | 0.05 | 1 | 6 | 66838539 | 7-[[(3S,9aS)-3,4,6,7,9,9a-hexahydro-1H-[1,4]oxazino[3,4-c][1,4]oxazin-3- |
| | | | | | yl]methoxy]-N-(3,4-dichloro-2-fluorophenyl)-6-methoxyquinazolin-4- |
| | | | | | amine |
| 0.05 | 0.05 | 1 | 6 | 66838467 | 7-[[(3S,9aS)-3,4,6,7,9,9a-hexahydro-1H-[1,4]oxazino[3,4-c][1,4]oxazin-3- |
| | | | | | yl]methoxy]-N-(4-bromo-5-chloro-2-fluorophenyl)-6-methoxyquinazolin- |
| | | | | | 4-amine |
| 0.05 | 0.05 | 1 | 6 | 66838463 | 7-[[(3S,9aS)-3,4,6,7,9,9a-hexahydro-1H-[1,4]oxazino[3,4-c][1,4]oxazin-3- |
| | | | | | yl]methoxy]-N-(4,5-dichloro-2-fluorophenyl)-6-methoxyquinazolin-4- |
| | | | | | amine |
| 0.004 | 0.004 | 1 | 7 | 11155938 | 5-[6-(5-Cyclopropylmethyl-[1,3,4]oxadiazol-2-yl)-5-isopropyl- |
| | | | | | pyrrolo[2,1-f][1,2,4]triazin-4-ylamino]-2,4-difluoro-N-methoxy- |
| | | | | | benzamide |
| 0.05 | 0.05 | 1 | 4 | 13414012 | 7-[[(3S,3aS,5R,6aR)-3-methoxy-2,3,3a,5,6,6a-hexahydrofuro[3,2-b]furan- |
| | | | | 6 | 5-yl]oxy]-N-(3,4-dichlorophenyl)-6-methoxyquinazolin-4-amine |
| 0.05 | 0.05 | 1 | 5 | 13414243 | 2-[[4-(3-chloro-2-fluoroanilino)-6-methoxyquinazolin-7-yl]oxymethyl]- |
| | | | | 8 | N′-cyano-1,4-oxazepane-4-carboximidamide |
| 0.0021 | 0.0021 | 1 | 7 | 11352215 | 2,4-Difluoro-5-[6-(5-isobutyl-[1,3,4]oxadiazol-2-yl)-5-isopropyl- |
| | | | | | pyrrolo[2,1-f][1,2,4]triazin-4-ylamino]-N-methoxy-benzamide |
| 0.0018 | 0.0018 | 1 | 7 | 11374515 | 5-[6-(5-Ethyl-[1,3,4]oxadiazol-2-yl)-5-isopropyl-pyrrolo[2,1- |
| | | | | | f][1,2,4]triazin-4-ylamino]-2,4-difluoro-N-methoxy-benzamide |
| 0.05 | 0.05 | 1 | 6 | 10346203 | 7-[[(3R,9aS)-3,4,6,7,9,9a-hexahydro-1H-[1,4]oxazino[3,4-c][1,4]oxazin-3- |
| | | | | | yl]methoxy]-N-(4,5-dichloro-2-fluorophenyl)-6-methoxyquinazolin-4- |
| | | | | | amine |
| 0.05 | 0.05 | 1 | 4 | 13415092 | 7-[[(3R,9aS)-1,3,4,6, 7,8,9,9a-octahydropyrido[2,1-c][1,4]oxazin-3- |
| | | | | 6 | yl]methoxy]-N-(4-bromo-2,3-dichlorophenyl)-6-methoxyquinazolin-4- |
| | | | | | amine |
| 0.05 | 0.05 | 1 | 4 | 66955051 | U.S. Pat. No. 9,796,704, Entry 42 |
| 0.046 | 0.046 | 1 | 7 | 155517777 | 1-N′-[4-[[5-fluoro-4-(7-fluoro-2-methyl-3-propan-2-ylbenzimidazol-5- |
| | | | | | yl)pyrimidin-2-yl]amino]phenyl]-1-N-(4-methylphenyl)cyclopropane-1,1- |
| | | | | | dicarboxamide |
| 0.0001 | 0.0001 | 1 | 4 | 6450551 | Axitinib |
| 0.003 | 0.003 | 1 | 3 | 73357210 | 2-Chloro-4-fluoro-5-[[6-methoxy-7-[(1-methylpiperidin-4- |
| | | | | | yl)methoxy\|quinazolin-4-yl]amino]phenol;hydrochloride |
| 0.008 | 0.008 | 1 | 3 | 73352657 | N-(4-chloro-2-fluorophenyl)-6-methoxy-7-(3-pyrrolidin-1- |
| | | | | | ylpropoxy)quinazolin-4-amine;hydrochloride |
| 0.001 | 0.001 | 1 | 2 | 9549295 | 1-(2-Chloro-4-(6,7-dimethoxyquinazolin-4-yloxy)phenyl)-3-propylurea |
| 0.003 | 0.003 | 1 | 5 | 71714235 | (3Z)-5-[(1-methylpiperidin-4-yl)amino]-3-[5-(morpholine-4-carbonyl)- |
| | | | | | 1,4,6,7-tetrahydropyrrolo[3,2-c]pyridin-2-yl]methylidene]-1H-indol-2-one |
| 0.069 | 0.069 | 1 | 5 | 9850161 | Unii-5ZH5NA7zmf |
| 0.006 | 0.006 | 1 | 5 | 71714231 | (3Z)-5-methoxy-3-[[5-(4-methylpiperazine-1-carbonyl)-1,4,6,7- |
| | | | | | tetrahydropyrrolo[3,2-c]pyridin-2-yl]methylidene]-1H-indol-2-one |
| 0.05 | 0.05 | 1 | 5 | 57802818 | 6-[[4-(3,4-Dichloroanilino)-6-methoxyquinazolin-7-yl]oxymethyl]-3,3,4- |
| | | | | | trimethylmorpholin-2-one |
| 0.05 | 0.05 | 1 | 6 | 9937516 | 7-[[(3S,9aS)-3,4,6,7,9,9a-hexahydro-1H-[1,4]oxazino[3,4-c][1,4]oxazin-3- |
| | | | | | yl]methoxy]-N-(4-bromo-3-chloro-2-fluorophenyl)-6-methoxyquinazolin- |
| | | | | | 4-amine |
| 0.0067 | 0.0067 | 1 | 7 | 9982235 | 5-[6-(5-Dimethylamino-[1,3,4]oxadiazol-2-yl)-5-isopropyl-pyrrolo[2,1- |
| | | | | | f][1,2,4]triazin-4-ylamino]-2,4-difluoro-N-methoxy-benzamide |
| 0.05 | 0.05 | 1 | 6 | 10028639 | 7-[[(3R,9aS)-3,4,6,7,9,9a-hexahydro-1H-[1,4]oxazino[3,4-c][1,4]oxazin-3- |
| | | | | | yl]methoxy]-N-(3-chloro-2,4-difluorophenyl)-6-methoxyquinazolin-4- |
| | | | | | amine |
| 0.05 | 0.05 | 1 | 6 | 10029284 | 7-[[(3R,9aS)-3,4,6,7,9,9a-hexahydro-1H-[1,4]oxazino[3,4-c][1,4]oxazin-3- |
| | | | | | yl]methoxy]-N-(3,4-dichloro-2-fluorophenyl)-6-methoxyquinazolin-4- |
| | | | | | amine |
| 0.05 | 0.05 | 1 | 6 | 10030541 | 7-[[(3R,9aS)-3,4,6,7,9,9a-hexahydro-1H-[1,4]oxazino[3,4-c][1,4]oxazin-3- |
| | | | | | yl]methoxy]-N-(4-bromo-5-chloro-2-fluorophenyl)-6-methoxyquinazolin- |
| | | | | | 4-amine |
| 0.017 | 0.017 | 1 | 7 | 10073832 | 5-[6-(5-Difluoromethyl-[1,3,4]oxadiazol-2-yl)-5-isopropyl-pyrrolo[2,1- |
| | | | | | f][1,2,4]triazin-4-ylamino]-2,4-difluoro-N-methoxy-benzamide |
| 0.04 | 0.04 | 1 | 4 | 5329054 | 4-Anilinoquinazoline 28 |
| 0.05 | 0.05 | 1 | 5 | 10253661 | U.S. Pat. No. 9,796,704, Entry 59 |
| 0.02 | 0.02 | 1 | 7 | 5329033 | Anilinoquinazoline deriv. 35 |
| 0.05 | 0.05 | 1 | 3 | 67163520 | 7-[[(3S,3aR,6S,6aS)-3-(diethylamino)-2,3,3a,5,6,6a-hexahydrofuro[3,2- |
| | | | | | b]furan-6-yl]oxy]-N-(4-bromo-3-chlorophenyl)-6-methoxyquinazolin-4- |
| | | | | | amine |
| 0.003 | 0.003 | 1 | 4 | 67162590 | 3-[(3,5-difluorophenyl)-(5-methyl-1H-imidazol-2-yl)methylidene]-5-[(1- |
| | | | | | ethylpiperidin-4-yl)amino]-1H-indol-2-one |
| 0.002 | 0.002 | 1 | 3 | 5329013 | Anilinoquinazoline deriv. 13 |
| 0.0068 | 0.0068 | 1 | 7 | 11419379 | 2,4-Difluoro-5-[5-isopropyl-6-(3-methyl-[1,2,4]oxadiazol-5-yl)- |
| | | | | | pyrrolo[2, 1-f][1,2,4]triazin-4-ylamino]-N-methoxy-benzamide |
| 0.05 | 0.05 | 1 | 5 | 66838558 | 7-[[(3S,9aS)-3,4,6,7,9,9a-hexahydro-1H-[1,4]oxazino[3,4-c][1,4]oxazin-3- |
| | | | | | yl]methoxy]-N-(4-bromo-2,3-dichlorophenyl)-6-methoxyquinazolin-4- |
| | | | | | amine |
| 0.027 | 0.027 | 1 | 7 | 11432911 | 5-{6-[5-(Difluoro-methanesulfonyl-methyl)-[1,3,4]oxadiazol-2-yl]-5- |
| | | | | | isopropyl-pyrrolo[2,1-f] [1,2,4]triazin-4-ylamino }-2,4-difluoro-N- |
| | | | | | methoxy-benzamide |
| 0.05 | 0.05 | 1 | 5 | 57802910 | N-(3,4-dichlorophenyl)-6-methoxy-7-[[2-(4-methylpiperazin-1-yl)-1,3- |
| | | | | | thiazol-4-yl]methoxy\|quinazolin-4-amine |
| 0.05 | 0.05 | 1 | 5 | 57802906 | 1-[2-[[4-(3,4-Dichloroanilino)-6-methoxyquinazolin-7- |
| | | | | | yl]oxymethyl]morpholin-4-yl]propan-1-one |
| 0.05 | 0.05 | 1 | 6 | 57802905 | U.S. Pat. No. 9,796,704, Entry 37 |
| 0.05 | 0.05 | 1 | 5 | 57802904 | [[2-[[4-(3,4-Dichloroanilino)-6-methoxyquinazolin-7- |
| | | | | | yl]oxymethyl]morpholin-4-yl]-pyrrolidin-1-ylmethylidene]cyanamide |
| 0.05 | 0.05 | 1 | 5 | 57802901 | N-(3,4-dichlorophenyl)-6-methoxy-7-[(4-methylsulfonylmorpholin-2- |
| | | | | | yl)methoxy]quinazolin-4-amine |
| 0.05 | 0.05 | 1 | 5 | 57802892 | (6S)-6-[[4-(4-bromo-3-chloro-2-fluoroanilino)-6-methoxyquinazolin-7- |
| | | | | | yl]oxymethyl]-4-methylpiperazin-2-one |
| 0.05 | 0.05 | 1 | 5 | 57802889 | N-(3,4-dichlorophenyl)-6-methoxy-7-[2-(4,5,5-trimethylmorpholin-2- |
| | | | | | yl)ethoxy]quinazolin-4-amine |
| 0.05 | 0.05 | 1 | 5 | 134155250 | N-(3,4-dichlorophenyl)-7-[(5-ethyl-4,6,7,7a-tetrahydro-3aH- |
| | | | | | [1,3]thiazolo[5,4-c]pyridin-2-yl)methoxy]-6-methoxyquinazolin-4-amine |
| 0.05 | 0.05 | 1 | 5 | 134151034 | N-(2,3-dichlorophenyl)-7-[(4-ethanimidoyl-1,4-oxazepan-2-yl)methoxy]- |
| | | | | | 6-methoxyquinazolin-4-amine |
| 0.05 | 0.05 | 1 | 5 | 57802916 | N-(4-bromo-2,5-difluorophenyl)-6-methoxy-7-[(4-methylmorpholin-2- |
| | | | | | yl)methoxy]quinazolin-4-amine |
| 0.05 | 0.05 | 1 | 6 | 57802880 | U.S. Pat. No. 9,796,704, Entry 31 |
| 0.05 | 0.05 | 1 | 5 | 57802871 | [2-[[4-(3,4-Dichloroanilino)-6-methoxyquinazolin-7- |
| | | | | | yl]oxymethyl]morpholin-4-yl]-(3,5-dimethyl-1,2-oxazol-4-yl)methanone |
| 0.05 | 0.05 | 1 | 5 | 57802868 | Cyclopentyl-[2-[[4-(3,4-dichloroanilino)-6-methoxyquinazolin-7- |
| | | | | | yl]oxymethyl]morpholin-4-yl]methanone |
| 0.05 | 0.05 | 1 | 5 | 57802867 | N-(4-bromo-3-chloro-2-methylphenyl)-6-methoxy-7-[(4- |
| | | | | | methylmorpholin-2-yl)methoxy]quinazolin-4-amine |
| 0.05 | 0.05 | 1 | 5 | 57802866 | U.S. Pat. No. 9,796,704, Entry 249 |
| 0.05 | 0.05 | 1 | 5 | 57802861 | U.S. Pat. No. 9,796,704, Entry 214 |
| 0.05 | 0.05 | 1 | 5 | 57802853 | N-(4,5-dichloro-2-fluorophenyl)-6-methoxy-7-[(4,5,5-trimethylmorpholin- |
| | | | | | 2-yl)methoxy]quinazolin-4-amine |
| 0.05 | 0.05 | 1 | 5 | 57802849 | N-(4-bromo-3,5-difluorophenyl)-6-methoxy-7-[(4-methylmorpholin-2- |
| | | | | | yl)methoxy]quinazolin-4-amine |
| 0.05 | 0.05 | 1 | 5 | 57802842 | N-(4-bromo-3-methylphenyl)-6-methoxy-7-[(4-methylmorpholin-2- |
| | | | | | yl)methoxy]quinazolin-4-amine |
| 0.05 | 0.05 | 1 | 5 | 57802833 | 7-[(4-butylmorpholin-2-yl)methoxy]-N-(3,4-dichlorophenyl)-6- |
| | | | | | methoxyquinazolin-4-amine |
| 0.05 | 0.05 | 1 | 5 | 57802878 | N′-cyano-2-[[4-(3,4-dichloroanilino)-6-methoxyquinazolin-7- |
| | | | | | yl]oxymethyl]morpholine-4-carboximidamide |
| 0.05 | 0.05 | 1 | 5 | 57802919 | U.S. Pat. No. 9,796,704, Entry 219 |
| 0.005 | 0.005 | 1 | 3 | 5330387 | 3-[[5-[6-[(3-Chlorophenyl)amino]pyrazinyl]-3-pyridinyl]amino]-1- |
| | | | | | propanol |
| 0.05 | 0.05 | 1 | 5 | 58013772 | U.S. Pat. No. 9,796,704, Entry 239 |
| 0.1 | 0.1 | 1 | 13 | 59756709 | N-hydroxy-4-[[[4-[4-[[(1R)-1-phenylethyl]amino]-7H-pyrrolo[2,3- |
| | | | | | d]pyrimidin-6-yl]phenyl]methylamino methyl]benzamide |
| 0.025 | 0.025 | 1 | 7 | 11455031 | 2,4-Difluoro-5-{5-isopropyl-6-[5-(2,2,2-trifluoro-ethyl)-[1,3,4]oxadiazol- |
| | | | | | 2-yl]-pyrrolo[2,1-f][1,2,4]triazin-4-ylamino}-N-methoxy-benzamide |
| 0.05 | 0.05 | 1 | 4 | 58013780 | N-(4-bromo-3-chlorophenyl)-6-methoxy-7-[(3R)-oxolan-3- |
| | | | | | yl]oxyquinazolin-4-amine |
| 0.0036 | 0.0036 | 1 | 7 | 11465545 | 5-[6-(5-Cyclopropyl-[1,3,4]oxadiazol-2-yl)-5-isopropyl-pyrrolo[2,1- |
| | | | | | f][1,2,4]triazin-4-ylamino]-2,4-difluoro-N-methoxy-benzamide |
| 0.05 | 0.05 | 1 | 5 | 58013796 | U.S. Pat. No. 9,796,704, Entry 238 |
| 0.05 | 0.05 | 1 | 4 | 134155171 | 7-[[(3S,3aS,5R,6aR)-3-methoxy-2,3,3a,5,6,6a-hexahydrofuro[3,2-b]furan- |
| | | | | | 5-yl]oxy]-N-(4-bromo-3-chloro-2-fluorophenyl)-6-methoxyquinazolin-4- |
| | | | | | amine |
| 0.05 | 0.05 | 1 | 6 | 58013804 | U.S. Pat. No. 9,796,704, Entry 25 |
| 0.046 | 0.046 | 1 | 4 | 11477626 | [4-(2,4-Difluoro-5-methoxycarbamoyl-phenylamino)-5-isopropyl- |
| | | | | | pyrrolo[2,1-f][1,2,4]triazin-6-yl]-carbamic acid tetrahydro-furan-2- |
| | | | | | ylmethyl ester |
| 0.05 | 0.05 | 1 | 5 | 134154844 | N-(4-bromo-5-chloro-2-fluorophenyl)-6-methoxy-7-[(4-methylmorpholin- |
| | | | | | 3-yl)methoxy]quinazolin-4-amine |
| 0.05 | 0.05 | 1 | 6 | 58013819 | U.S. Pat. No. 9,796,704, Entry 38 |

### Column Key

VEGFR2 IC50_um: reported IC₅₀ to VEGFR2 in μM.

Min IC50_um: next lowest IC₅₀ value to any gene target in μM.

Relative IC50: IC₅₀ of target of interest divided by next lowest IC50

Num low IC50 Targets: the number of gene targets that have a reported IC₅₀ within 10-fold of the IC₅₀ to the target of interest (VEGFR2 or FGFR1).

Pubchem_cid: Pubchem chemical ID

Compound name: Name of compound

TABLE 4
Brivanib and SU5402

| Compound | Structure |
|---|---|
| Brivanib alaninate (Brivanib) | 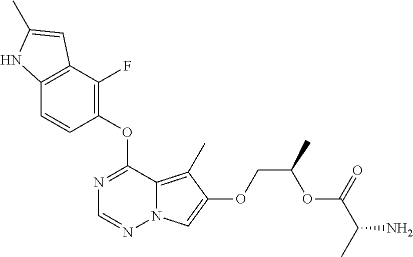 [SMILES: Cc1cc2c(F)c(Oc3ncnn4cc(OC[C@@H](C)OC(=O)[C@@H](C)N)c(C)c34)ccc2[nH]1] |
| 3-[(3-(2-Carboxyethyl)-4- methylpyrrol-2-YL)methylene]- 2-indolinone (SU5402) | 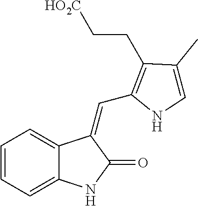 [SMILES: Cc1c[nH]c(/C=C2\C(=O)Nc3ccccc32)c1CCC(=O)O] |

### Incorporation by Reference

The entire disclosure of each of the patent documents and scientific articles cited herein is incorporated by reference for all purposes.

1. A method of preserving or promoting lipid storage in subcutaneous adipose tissue, fat mass increase, brown fat mass increase, bone density, lean muscle mass increase, the thickening of abdominal subcutaneous adipose tissue, or decreasing liver lipid content in a subject comprising administering to the subject a selective inhibitor of VEGFR2 and FGFR1, wherein the administration promotes the lipid storage or fat mass increase in the subject as compared to administration of a control agent or as compared to historical data obtained from the subject prior to the administration of the selective inhibitor of VEGFR2 and FGFR1.

2-10. (canceled)

11. The method of claim 1, wherein the wasting syndrome is selected from the group consisting of lipoatrophy, cachexia, liver disease, kidney disease, fatty liver disease, Non-alcoholic Steato Hepatitis (NASH), diabetes, osteoporosis, Paget's disease of the bone, osteoarthritis, rheumatoid arthritis, sarcopenia, and frailty.

12-24. (canceled)

25. A method of treating, preventing, reversing, or delaying progression of a wasting syndrome in a subject comprising administering to the subject a selective inhibitor of VEGFR2 and FGFR1.

26. The method of claims 25, comprising treating, reversing, or delaying progression of the wasting syndrome in the subject.

27. The method of claim 25, wherein the wasting syndrome is selected from the group consisting of lipoatrophy, cachexia, liver disease, kidney disease, fatty liver disease, Non-alcoholic Steato Hepatitis (NASH), diabetes, osteoporosis, Paget's disease of the bone, osteoarthritis, rheumatoid arthritis, sarcopenia, and frailty.

28-40. (canceled)

41. The method of claim 1, wherein the selective inhibitor of inhibitor of VEGFR2 and FGFR1 is administered as a mono-drug therapy.

42. The method of claim 1, wherein the selective VEGFR2 and FGFR1 inhibitor is administered 6 times/day, 4 times/day, 3 times/day, 2 times/day, once per day, once every 2 days, once every 3 days, once every 4 days, once every 5 days, once every 6 days, or once per week.

43. The method of claim 1, wherein the selective VEGFR2 and FGFR1 inhibitor is selected from the group consisting of [4-(2,4-Difluoro-5-methoxycarbamoyl-phenylamino)-5-isopropyl-pyrrolo[2,1-f][1,2,4]triazin-6-yl]-carbamic acid tetrahydro-furan-2-ylmethyl ester, Brivanib Alaninate (Brivanib), 2,4-Difluoro-5-[5-isopropyl-6-(5-trifluoromethyl-[1,3,4]oxadiazol-2-yl)-pyrrolo[2,1-f][1,2,4]triazin-4-ylamino]-N-methoxy-benzamide, 5-[(1-Ethylpiperidin-4-yl)amino]-3-[1H-imidazol-2-yl(phenyl)methylidene]-1H-indol-2-one, 2,4-Difluoro-5-[5-isopropyl-6-(5-methyl-4H-[1,2,4]triazol-3-yl)-pyrrolo[2,1-f][1,2,4]triazin-4-ylamino]-N-methoxy-benzamide, 5-[6-(5-Dimethylamino-[1,3,4]oxadiazol-2-yl)-5-isopropyl-pyrrolo[2,1-f][1,2,4]triazin-4-ylamino]-2,4-difluoro-N-methoxy-benzamide, 2,4-Difluoro-5-[5-isopropyl-6-(3-methyl-[1,2,4]oxadiazol-5-yl)-pyrrolo[2,1-f][1,2,4]triazin-4-ylamino]-N-methoxy-benzamide, 3-[(3-(2-Carboxyethyl)-4-methylpyrrol-2-YL)methylene]-2-indolinone (SU5402), 2,4-Difluoro-5-[5-isopropyl-6-(5-methyl-[1,3,4]oxadiazol-2-yl)-pyrrolo[2,1-f][1,2,4]triazin-4-ylamino]-N-methoxy-benzamide, 5-[6-(5-Difluoromethyl-[1,3,4]oxadiazol-2-yl)-5-isopropyl-pyrrolo[2,1-f][1,2,4]triazin-4-ylamino]-2,4-difluoro-N-methoxy-benzamidem, 3-[Benzimidazol-2-ylidene-(3-fluorophenyl)methyl]-5-[(1-ethylpiperidin-4-yl)amino]-1H-indol-2-ol, N-(Cyclopropylmethyl)-3-(1-methylpyrazol-4-yl)-N-phenylquinoxalin-6-amine, 2,4-Difluoro-5-(5-isopropyl-6-[1,3,4]oxadiazol-2-yl-pyrrolo[2,1-f][1,2,4]triazin-4-ylamino)-N-methoxy-benzamide, 7-(4-Fluoroanilino)-1-[(1R,3R)-3-hydroxycyclopentyl]-3-(4-methoxyphenyl)-4H-pyrimido[4,5-d]pyrimidin-2-one, 5-[6-(5-Cyclopropyl-[1,3,4]oxadiazol-2-yl)-5-isopropyl-pyrrolo[2,1-f][1,2,4]triazin-4-ylamino]-2,4-difluoro-N-methoxy-benzamide, 6-(5-Methyl-1,3,4-oxadiazol-2-yl)-5-propan-2-yl-N-(1H-pyrrolo[2,3-b]pyridin-5-yl)pyrrolo[2,1-f][1,2,4]triazin-4-amine, N-(Cyclopropylmethyl)-N-[3-methoxy-5-(trifluoromethyl)phenyl]-3-(1-methylpyrazol-4-yl)quinoxalin-6-amine, 3-[Benzimidazol-2-ylidene-(3,5-difluorophenyl)methyl]-5-[(1-ethylpiperidin-4-yl)amino]-1H-indol-2-ol, 1,6-Naphthyridine 76, 2,4-Difluoro-5-{5-isopropyl-6-[5-(2,2,2-trifluoro-ethyl)-[1,3,4]oxadiazol-2-yl]-pyrrolo[2,1-f][1,2,4]triazin-4-ylamino}-N-methoxy- benzamide, (+)-(1R,3R)-1-(3-Hydroxy-cyclopentyl)-3-(4-methoxy-phenyl)-7-phenylamino-3,4-dihydro-1H-pyrimido[4,5-d]pyrimidin-2-one, 1,6-Naphthyridine 75, (−)-(1S,3S)-1-(3-Hydroxy-cyclopentyl)-3-(4-methoxy-phenyl)-7-phenylamino-3,4-dihydro-1H-pyrimido[4,5-d]pyrimidin-2-one, N-(Cyclopropylmethyl)-N-[3-[(dimethylamino)methyl]-5-methoxyphenyl]-3-(1-methylpyrazol-4-yl)quinoxalin-6-amine, N-[3-(Aminomethyl)-5-methoxyphenyl]-N-(cyclopropylmethyl)-3-(1-methylpyrazol-4-yl)quinoxalin-6-amine;hydrochloride, Chemb14573547, 1,6-Naphthyridine 77, 1,6-Naphthyridine 19, 1,6-Naphthyridine 26, N-(Cyclopropylmethyl)-N-[3-(difluoromethoxy)phenyl]-3-(1-methylpyrazol-4-yl)quinoxalin-6-amine, N′-(3,4-Difluoro-5-methoxyphenyl)-N′-[3-(1-methylpyrazol-4-yl)quinoxalin-6-yl]-N-(2,2,2-trifluoroethyl)propane-1,3-diamine;hydrochloride, 2,4-Difluoro-5-[5-isopropyl-6-(5-methanesulfonylmethyl-[1,3,4]oxadiazol-2-yl)-pyrrolo[2,1-f][1,2,4]triazin-4-ylamino]-N-methoxy-benzamide, 5-[2-Chlorophenyl)-7-fluoro-1,2-dihydro-8-methoxy-3-methylpyrazolo(3,4-b)(1,4)benzodiazepine, [3-[Cyclopropylmethyl-[3-(1-methylpyrazol-4-yl)quinoxalin-6-yl]amino]-5-methoxyphenyl]-pyrrolidin-1-ylmethanone, 5-[(1-Ethylpiperidin-4-yl)amino]-3-[(2-fluorophenyl)-(5-methyl-1H-imidazol-2-yl)methylidene]-1H-indol-2-one, 5-[6-(5-Cyclopropylmethyl-[1,3,4]oxadiazol-2-yl)-5-isopropyl-pyrrolo[2,1-f][1,2,4]triazin-4-ylamino]-2,4-difluoro-N-methoxy-benzamide, 3-[(3,5-Difluorophenyl)-(5-methyl-1H-imidazol-2-yl)methylidene]-5-[(1-ethylpiperidin-4-yl)amino]-1H-indol-2-one, 5-[(1-Ethylpiperidin-4-yl)amino]-3-[(3-fluorophenyl)-(1H-imidazol-2-yl)methylidene]-1H-indol-2-one, Indolin-2-one deriv. 9c, Methyl (3Z)-2-oxo-3-[phenyl-[4-(piperidin-1-ylmethyl)anilino]methylidene]-1H-indole-6-carboxylate, N-(3,5-Dimethoxyphenyl)-N-[3-(3-methylimidazol-4-yl)prop-2-ynyl]-3-(1-methylpyrazol-4-yl)quinoxalin-6-amine, (3z)-5-[(1-Ethylpiperidin-4-Yl)amino]-3-[(5-Methoxy-1h-Benzimidazol-2-Yl)(Phenyl)methylidene]-1,3-Dihydro-2h-Indol-2-One, [5-Amino-1-(1H-indo1-5-yl)pyrazol-4-yl]-(1H-indo1-2-yl)methanone, 5-Isopropyl-6-(5-methyl-1,3,4-oxadiazol-2-yl)-N-(2-methyl-1H-pyrrolo[2,3-b]pyridin-5-yl)pyrrolo[2,1-f][1,2,4]triazin-4-amine, 3-[Benzimidazol-2-ylidene-(4-methylphenyl)methyl]-5-[(1-ethylpiperidin-4-yl)amino]-1H-indol-2-ol, 5-[6-(5-Ethyl-[1,3,4]oxadiazol-2-yl)-5-isopropyl-pyrrolo[2,1-f][1,2,4]triazin-4-ylamino]-2,4-difluoro-N-methoxy-benzamide, 3-[Benzimidazol-2-ylidene(phenyl)methyl]-5-[(1-ethylpiperidin-4-yl)amino]-1H-indol-2-ol, 1,6-Naphthyridine 25, 2-(6-Fluoro-1H-indazol-3-yl)-5-(4-(piperidin-1-yl)piperidin-1-yl)-1H-benzo[d]imidazole, 3-[Benzimidazol-2-ylidene-(4-methoxyphenyl)methyl]-5-[(1-ethylpiperidin-4-yl)amino]-1H-indol-2-ol, N-(Cyclopropylmethyl)-N-P-methoxy-5-[2-(methylamino)ethoxy]phenyl]-3-(1-methylpyrazol-4-yl)quinoxalin-6-amine, 3-Methoxy-N-methyl-5-[[3-(1-methylpyrazol-4-yl)quinoxalin-6-yl]-[2-(propan-2-ylamino)ethyl]amino]benzamide, XL999;XL-999; XL 999, 3-N-(Cyclopropylmethyl)-1-N-methyl-3-N-[3-(1-methylpyrazol-4-yl)quinoxalin-6-yl]benzene-1,3-diamine, 2,4-Difluoro-5-[6-(5-isobutyl-[1,3,4]oxadiazol-2-yl)-5-isopropyl-pyrrolo[2,1-f][1,2,4]triazin-4-ylamino]-N-methoxy-benzamide, Methyl 2-hydroxy-3-[N-[4-[2-methyl-5-[(4-methylpiperazin-1-yl)methyl]pyrrol-1-yl]phenyl]-C-phenylcarbonimidoyl]-1H-indole-6-carboxylate, N-(3,5-Dimethoxyphenyl)-3-(1-methylpyrazol-4-yl)-N-[(2-pyridin-4-ylcyclopropyl)methyl]quinoxalin-6-amine, 3-(3,5-Dimethoxyphenyl)-7-N-[3-(4-methylpiperazin-1-yl)propyl]-1,6-naphthyridine-2,7-diamine, 9-Chloro-2-[3-[3-(dimethylamino)propyl]anilino]-5,7-dihydropyrimido[5,4-d][1]benzazepine-6-thione, Pyrido[2,3-d]pyrimidine 103, Pyrido[2,3-d]pyrimidine 105, 2,4-Difluoro-5-[5-isopropyl-6-(5-methyl-oxazol-2-yl)-pyrrolo[2,1-f][1,2,4]triazin-4-ylamino]-N-methoxy-benzamide, N-(3,5-Dimethoxyphenyl)-N-[3-(2,5-dimethylimidazol-1-yl)propyl]-3-(1-methylpyrazol-4-yl)quinoxalin-6-amine;hydrochloride, N-[3-[2-[6-(2-Chlorophenyl)-2-[4-(diethylamino)butylamino]-7-oxopyrido[2,3-d]pyrimidin-8-yl]ethyl]phenyl]prop-2-enamide, N-(3,5-Dimethoxyphenyl)-3-(1-methylpyrazol-4-yl)-N-(2-piperazin-1-ylethyl)quinoxalin-6-amine;hydrochloride, Methyl (3Z)-3-[[4-[2-(dimethylamino)ethyl-methylsulfonylamino]anilino]-phenylmethylidene]-2-oxo-1H-indole-6-carboxylate, 4-[3-(3,5-Dimethoxy-N-[3-(1-methylpyrazol-4-yl)quinoxalin-6-yl]anilino)prop-1-ynyl]pyridine-3-carbonitrile, N-[(5-Chlorothiophen-2-yl)methyl]-N-(3,5-dimethoxyphenyl)-3-[1-(2-piperazin-1-ylethyl)pyrazol-4-yl]quinoxalin-6-amine;hydrochloride, N′-(3,5-Dichlorophenyl)-N′-[3-(1-methylpyrazol-4-yl)quinoxalin-6-yl]-N-(2,2,2-trifluoroethyl)propane-1,3-diamine, 2-[4-[7-[N-(Cyclopropylmethyl)anilino]quinoxalin-2-yl]pyrazol-1-yl]ethanol, Pyrido[2,3-d]pyrimidine 102, 7-N-[4-(Diethylamino)butyl]-3-(3,5-dimethoxyphenyl)-1,6-naphthyridine-2,7-diamine, N-(3,5-Dimethoxyphenyl)-N-[3-(4-methoxypyrimidin-2-yl)propyl]-3-(1-methylpyrazol-4-yl)quinoxalin-6-amine, Methyl 3-[[4-[(dimethylamino)methyl]anilino]-phenylmethylidene]-2-oxo-1H-indole-6-carboxylate, 3-Fluoro-N-methyl-5-[[3-(1-methylpyrazol-4-yl)quinoxalin-6-yl]-[2-(propan-2-ylamino)ethyl]amino]benzamide, N-(Cyclopropylmethyl)-N-[3-(1,3-dioxolan-2-yl)-5-methoxyphenyl]-3-(1-methylpyrazol-4-yl)quinoxalin-6-amine, N-[[(2S)-1-[3-(3,5-Dimethoxy-N-[3-(1-methylpyrazol-4-yl)quinoxalin-6-yl]anilino)propyl]pyrrolidin-2-yl]methyl]-1,1,1-trifluoromethanesulfonamide, 3-Methoxy-N-methyl-5-[2-(propan-2-ylamino)ethyl-[3-(1-propan-2-ylpyrazol-4-yl)quinoxalin-6-yl]amino]benzamide, 5-{6-[5-(Difluoro-methanesulfonyl-methyl)-[1,3,4]oxadiazol-2-yl]-5-isopropyl-pyrrolo[2,1-f][1,2,4]triazin-4-ylamino}-2,4-difluoro-N-methoxy- benzamide, N-[2-(3-Aminopiperidin-1-yl)ethyl]-N-(3,5-dimethoxyphenyl)-3-(1-methylpyrazol-4-yl)quinoxalin-6-amine, N-(3,5-Dimethoxyphenyl)-N-[(E)-3-(4-methoxypyrimidin-2-yl)prop-2-enyl]-3-(1-methylpyrazol-4-yl)quinoxalin-6-amine, Methyl (3Z)-3-[[4-[acetyl-[2-(dimethylamino)ethyl]amino]anilino]-phenylmethylidene]-2-oxo-1H-indole-6-carboxylate, Methyl N-[4-[7-amino-3-(3,4-dimethoxyphenyl)-5-(1-methylpiperidin-4-yl)oxypyrazolo[1,5-a]pyrimidin-6-yl]phenyl]carbamate, Anilinoquinazoline deriv. 35, 1,6-Naphthyridine 17, Tert-butyl (1S,4S)-5-[2-(3,5-dimethoxy-N-[3-(1-methylpyrazol-4-yl)quinoxalin-6-yl]anilino)ethyl]-2,5-diazabicyclo[2.2.1]heptane-2-carboxylate, Lucitanib, 6-((7-((1-Aminocyclopropyl)methoxy)-6-methoxyquinolin-4-yl)oxy)-N-methyl-1-naphthamide hydrochloride, Pyrido[2,3-d]pyrimidine 104, Nintedanib esylate, [(2S)-1-(3,5-Dimethoxy-N-[3-(1-methylpyrazol-4-yl)quinoxalin-6-yl]anilino)-3-methoxypropan-2-yl] acetate, 3-[2-Aminoethyl-[3-(1-methylpyrazol-4-yl)quinoxalin-6-yl]amino]-5-methoxy-N-methylbenzamide, N-(3,5-Dimethoxyphenyl)-3-(1-methylpyrazol-4-yl)-N-[3-[4-(trifluoromethyl)piperidin-1-yl]propyl]quinoxalin-6-amine, 3-((4-Bromo-2,6-difluorobenzyl)oxy)-5-(3-(4-(pyrrolidin-1-yl)butypureido)isothiazole-4-carboxamide, N-(3,5-Dimethoxyphenyl)-N-[3-(2-methylimidazol-1-yl)propyl]-3-(1-methylpyrazol-4-yl)quinoxalin-6-amine, 3-[3-(Morpholinomethyl)-4,5,6,7-tetrahydro-1H-indole-2-ylmethylene]-5-(ethylsulfonyl)-2,3-dihydro-1H-indole-2-one, 2-(3-Methoxy-N-[3-(1-methylpyrazol-4-yl)quinoxalin-6-yl]anilino)ethanol, N-[3-(5-Aminopyrazin-2-yl)prop-2-ynyl]-N-(3,5-dimethoxyphenyl)-3-(1-methylpyrazol-4-yl)quinoxalin-6-amine, 2-[3-[2-Aminoethyl-[7-(1-methylpyrazol-4-yl)quinoxalin-2-yl]amino]-5-fluorophenoxy]ethanol, N-[3-(3-Aminopyridin-2-yl)prop-2-ynyl]-N-(3,5-dimethoxyphenyl)-3-(1-methylpyrazol-4-yl)quinoxalin-6-amine, N-(3,5-Dimethoxyphenyl)-N-[3-(3-methoxypyridin-2-yl)propyl]-3-(1-methylpyrazol-4-yl)quinoxalin-6-amine, N-(3,5-Dimethoxyphenyl)-3-(1-methylpyrazol-4-yl)-N-[(Z)-3-pyrimidin-2-ylprop-2-enyl]quinoxalin-6-amine, N′-(3,5-Dimethoxyphenyl)-N-methyl-N′-[3-[1-(oxan-4-ylmethyl)pyrazol-4-yl]quinoxalin-6-yl]ethane-1,2-diamine;hydrochloride, [5-Amino-1-[2-chloro-5-hydroxyphenyl)pyrazol-4-yl]-[5-(morpholin-4-ylmethyl)-1H-indol-2-yl]methanone, 1,6-Naphthyridine 18, 1-Tert-butyl-3-[3-(3,5-dimethoxyphenyl)-7-[4-(4-methylpiperazin-1-yl)butylamino]-1,6-naphthyridin-2-yl]urea, N′-(2-Chloro-3,5-dimethoxyphenyl)-N′-[3-[1-methylpyrazo 1-4-yl)quinoxalin-6-yl]-N-propan-2-ylethane-1,2-diamine;hydrochloride, N-(3,5-Dimethoxyphenyl)-3-(1-methylpyrazol-4-yl)-N-(3-piperazin-1-ylpropyl)quinoxalin-6-amine, Methyl (3Z)-3-[[4-[acetyl-[3-(dimethylamino)propyl]amino]anilino]-phenylmethylidene]-2-oxo-1H-indole-6-carboxylate, 3-[4-[3-[4-[7-[N-(Cyclopropylmethyl)-3,5-dimethoxyanilino] quinoxalin-2-yl]pyrazol-1-yl]propyl]piperazin-1-yl]propan-1-ol;hydrochloride, 5-[(R)-1-(3,5-Dichloropyridine-4-yl)ethoxy]-3-[5-(4-methylpiperazine-1-yl)-1H-benzoimidazole-2-yl]-1H-indazole, 4-Amino-3-(1H-benzo [d]imidazol-2-yl)quinolin-2(1H)-one, 3-[4-[3-[4-[7-[3,5-Dimethoxy-N-[2-(propan-2-ylamino)ethyl]anilino]quinoxalin-2-yl]pyrazol-1-yl]propyl]piperazin-1-yl]propan-1-ol;hydrochloride, 2-(2,6-Difluoro-3,5-dimethoxy-N-[3-(1-methylpyrazol-4-yl)quinoxalin-6-yl]anilino)ethanol, [4-[7-[3,5-Dimethoxy-N-[2-(propan-2-ylamino)ethyl]anilino]quinoxalin-2-yl]-2-methylpyrazol-3-yl]methanol, 1,6-Naphthyridine deriv. 20, N-(2-Chloro-3,5-dimethoxyphenyl)-3-(1-methylpyrazol-4-yl)-N-(3-pyrrolidin-1-ylpropyl)quinoxalin-6-amine;hydrochloride, Methyl 3-[N-[4-[2-(dimethylamino)ethyl-methylcarbamoyl]phenyl]-C-phenylcarbonimidoyl]-2-hydroxy-1H-indole-6-carboxylate, N-[2-(1,4-Diazepan-1-yl)ethyl]-N-(3,5-dimethoxyphenyl)-3-(1-methylpyrazol-4-yl)quinoxalin-6-amine;oxalic acid, N′-(2,6-Difluoro-3,5-dimethoxyphenyl)-N′-[3-[1-methylpyrazol-4-yl)quinoxalin-6-yl]-N-propan-2-ylethane-1,2-diamine, N-(3,5-Dimethoxyphenyl)-N-[(3-methyloxetan-3-yl)methyl]-3-(1-methylpyrazol-4-yl)quinoxalin-6-amine, Methyl (3Z)-3-[[4-[acetyl-[2-(dimethylamino)-2-oxoethyl]amino]anilino]-phenylmethylidene]-2-oxo-1H-indole-6-carboxylate, Chemb14213341, Anilinoquinazoline deriv. 36, [5-Amino-1-(1H-benzimidazol-6-yl)-1H-pyrazol-4-yl]-1H-indol-2-ylMethanone, Hematoxylone, N-Cyclopropyl-N′-(3,5-dimethoxyphenyl)-N′-[3-(1-methylpyrazol-4-yl)quinoxalin-6-yl]ethane-1,2-diamine, N-(3,5-Dimethoxyphenyl)-3-(1-methylpyrazol-4-yl)-N-(3-pyrimidin-2-ylpropyl)quinoxalin-6-amine, N′-(3,5-Dimethoxyphenyl)-N′-[3-[5-[(dimethylamino)methyl]-1-methylpyrazol-4-yl]quinoxalin-6-yl]-N-propan-2-ylethane-1,2-diamine, N′-(2,6-Difluoro-3,5-dimethoxyphenyl)-N′-[3-(1-methylpyrazol-4-yl)quinoxalin-6-yl]-N-(2,2,2-trifluoroethyl)propane-1,3-diamine, [5-Amino-1-(2-methyl-1H-indol-5-yl)pyrazol-4-yl]-(1H-indol-2-yl)methanone, Haematoxylin, N′-(3,5-Dimethoxyphenyl)-N′-[3-(5-ethyl-1-methylpyrazol-4-yl)quinoxalin-6-yl]-N-(2,2,2-trifluoroethyl)ethane-1,2-diamine, Methyl 3-[N-[4-[[dimethylcarbamoyl(methyl)amino]methyl]phenyl]-C-phenylcarbonimidoyl]-2-hydroxy-1H-indole-6-carboxylate, 3-Methoxy-N-methyl-5-[[3-(1-methylpyrazol-4-yl)quinoxalin-6-yl]-(3-pyrrolidin-1-ylpropyl)amino]benzamide, N-(3,5-Dimethoxyphenyl)-3-(1-methylpyrazol-4-yl)-N-(3-pyridin-4-ylpropyl)quinoxalin-6-amine, 1-[3-(3,5-Dimethoxy-N-[3-(1-propan-2-ylpyrazol-4-yl)quinoxalin-6-yl]anilino)propyl]pyrrolidin-2-one, and Regorafenib.

44. The method of claim 43, wherein the selective VEGFR2 and FGFR1 inhibitor is Brivanib Alaninate (Brivanib).

45. The method of claim 44, wherein Brivanib Alaninate is administered to the subject at a daily dose of 10 mg/kg-200 mg/kg.

46. The method of claim 45, wherein Brivanib Alaninate is administered to the subject at a daily dose of 25 mg/kg.

47. The method of claim 45, wherein Brivanib Alaninate is administered to the subject at a daily dose of 50 mg/kg.

48. The method of claim 1, wherein the selective VEGFR2 and FGFR1 inhibitor is SU5402.

49. The method of claim 48, wherein SU5402 is administered to the subject at a daily dose of 5 mg/kg-100 mg/kg.

50. The method of claim 49, wherein SU5402 is administered to the subject at a daily dose of 25 mg/kg.

51. The method of claim 1, wherein the selective VEGFR2 and FGFR1 inhibitor is administered for up to 3 months.

52. The method of claim 1, wherein the selective VEGFR2 and FGFR1 inhibitor is administered for up to 2 months.

53. The method of claim 1, wherein the selective VEGFR2 and FGFR1 inhibitor is administered for up to 1 month.

54. The method of claim 1, wherein the selective VEGFR2 and FGFR1 inhibitor is administered by a route selected from the group consisting of topically, intrathecally, intrathalamically, intracisternally, parenterally, orally, rectally, buccally, sublingually, pulmonarily, intratracheally, intranasally, transdermally, and intraduodenally.
